SEQUENCE LISTING (56 sequences)

SEQ ID NO: 1 - protein, 12 aa, Homo sapiens
  Spacer (IgG4hinge)
ESKYGPPCPPCP

SEQ ID NO: 2 - DNA, 36 bp, Homo sapiens
  Spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28
FWVLVVVGGVLACYSLLVTVAFIIFWV

SEQ ID NO: 9 - protein, 66 aa, Homo sapiens
  CD28
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28
RSKRSRLLHSDYMNMTPRRPGPTRKHYQPYAPPRDFAAYRS

SEQ ID NO: 11 - protein, 41 aa, Homo sapiens
  CD28
RSKRSRGGHSDYMNMTPRRPGPTRKHYQPYAPPRDFAAYRS

SEQ ID NO: 12 - protein, 42 aa, Homo sapiens
  4-1BB
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 10 aa, Artificial Sequence
  Linker
  REPEAT (5)...(9): SGGGG is repeated 5 times
PGGGSGGGGP

SEQ ID NO: 23 - protein, 17 aa, Artificial Sequence
  Linker
GSADDAKKDAAKKDGKS

SEQ ID NO: 24 - protein, 18 aa, Artificial Sequence
  Linker
GSTSGSGKPGSGEGSTKG

SEQ ID NO: 25 - DNA, 735 bp, Artificial Sequence
  scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 26 - protein, 5 aa, Artificial Sequence
  Hinge
  VARIANT (1)...(1): Xaa1 is glycine, cysteine or arginine
  VARIANT (4)...(4): Xaa4 is cysteine or threonine
XPPXP

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 12 aa, Artificial Sequence
  Hinge
ESKYGPPCPPCP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  FMC63 CDR L1
RASQDISKYLN

SEQ ID NO: 36 - protein, 7 aa, Artificial Sequence
  FMC63 CDR L2
SRLHSGV

SEQ ID NO: 37 - protein, 9 aa, Artificial Sequence
  FMC63 CDR L3
GNTLPYTFG

SEQ ID NO: 38 - protein, 5 aa, Artificial Sequence
  FMC63 CDR H1
DYGVS

SEQ ID NO: 39 - protein, 16 aa, Artificial Sequence
  FMC63 CDR H2
VIWGSETTYYNSALKS

SEQ ID NO: 40 - protein, 7 aa, Artificial Sequence
  FMC63 CDR H3
YAMDYWG

SEQ ID NO: 41 - protein, 120 aa, Artificial Sequence
  FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  FMC63 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  SJ25C1 CDR L1
KASQNVGTNVA

SEQ ID NO: 45 - protein, 7 aa, Artificial Sequence
  SJ25C1 CDR L2
SATYRNS

SEQ ID NO: 46 - protein, 9 aa, Artificial Sequence
  SJ25C1 CDR L3
QQYNRYPYT

SEQ ID NO: 47 - protein, 5 aa, Artificial Sequence
  SJ25C1 CDR H1
SYWMN

SEQ ID NO: 48 - protein, 17 aa, Artificial Sequence
  SJ25C1 CDR H2
QIYPGDGDTNYNGKFKG

SEQ ID NO: 49 - protein, 13 aa, Artificial Sequence
  SJ25C1 CDR H3
KTISSVVDFYFDY

SEQ ID NO: 50 - protein, 122 aa, Artificial Sequence
  SJ25C1 VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  SJ25C1 VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, Artificial Sequence
  SJ25C1 scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  FMC63 CDR H3
HYYYGGSYAMDY

SEQ ID NO: 55 - protein, 7 aa, Artificial Sequence
  FMC63 CDR L2
HTSRLHS

SEQ ID NO: 56 - protein, 9 aa, Artificial Sequence
  FMC63 CDR L3
QQGNTLPYT

(19) United States
(12) Patent Application Publication
(10) Pub. No.: US 2019/0358262 A1
(43) Pub. Date: Nov. 28, 2019

(54) METHODS FOR MODULATION OF CAR-T CELLS

(71) Applicant: Juno Therapeutics, Inc., Seattle, WA (US)

(72) Inventor: Tina ALBERTSON, Seattle, WA (US)

(73) Assignee: Juno Therapeutics, Inc., Seattle, WA (US)

(21) Appl. No.: 16/465,520

(22) PCT Filed: Dec. 1, 2017
(86) PCT No.: PCT/US2017/064363

§ 371 Date: May 30, 2019

Related U.S. Application Data
(60) Provisional application No. 62/580,414, filed on Nov. 1, 2017.
(60) Provisional application No. 62/549,391, filed on Aug. 23, 2017.
(60) Provisional application No. 62/515,512, filed on Jun. 5, 2017.
(60) Provisional application No. 62/514,777, filed on Jun. 2, 2017.
(60) Provisional application No. 62/492,950, filed on May 1, 2017.
(60) Provisional application No. 62/444,784, filed on Jan. 10, 2017.
(60) Provisional application No. 62/429,740, filed on Dec. 3, 2016.

(51) Int. Cl. A61K 35/17; C07K 16/28; A61K 31/454; A61P 35/00; A61B 10/02; A61B 18/02; A61N 5/10

(52) U.S. Cl. CPC A61K 35/17; C07K 16/2827; A61K 31/454; A61P 35/00; A61B 10/0233; A61B 2018/00577; A61B 10/0283; A61B 18/02; A61N 5/10; A61N 5/1014; A61B 10/0266

(57) ABSTRACT

Provided herein are methods of modulating, in vivo, cells engineered with a recombinant receptor, such as a T cell receptor (TCR) or chimeric antigen receptor (CAR). In some embodiments, the methods include disrupting an area in the subject in which the cells are present or likely to be present or were present or were likely to be present, such as a lesion, including a tumor. In some embodiments, the disruption alters the environment of the lesion, e.g. tumor microenvironment. In some embodiments, the disruption is a biopsy. In some aspects, the provided methods result in increased expansion, and, in some cases, a more robust and durable response, of the engineered cells after carrying out the disruption.

### CROSS-REFERENCE TO RELATED APPLICATIONS

This application claims priority from U.S. provisional application No. 62/429,740, filed Dec. 3, 2016, entitled “METHODS FOR MODULATION OF GENETICALLY ENGINEERED CELLS,” U.S. provisional application No. 62/444,784, filed Jan. 10, 2017, entitled “METHODS FOR MODULATION OF GENETICALLY ENGINEERED CELLS,” U.S. provisional application No. 62/492,950, filed May 1, 2017 entitled “METHODS FOR MODULATION OF GENETICALLY ENGINEERED CELLS,” U.S. provisional application No. 62/514,777, filed Jun. 2, 2017 entitled “METHODS FOR MODULATION OF GENETICALLY ENGINEERED CELLS,” U.S. provisional application No. 62/515,512, filed Jun. 5, 2017, entitled “METHODS FOR MODULATION OF GENETICALLY ENGINEERED CELLS,” U.S. provisional application No. 62/549,391, filed Aug. 23, 2017, entitled “METHODS FOR MODULATION OF GENETICALLY ENGINEERED CELLS,” and U.S. provisional application No. 62/580,414, filed Nov. 1, 2017, entitled “METHODS FOR MODULATION OF GENETICALLY ENGINEERED CELLS,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042009140SeqList.txt, created Nov. 29, 2017, which is 34,855 kilobytes in size. The information in the electronic format of the Sequence Listing is incorporated by reference in its entirety.

### FIELD

The present disclosure relates in some aspects to a method of modulating, in vivo, cells engineered with a recombinant receptor, such as a T cell receptor (TCR) or chimeric antigen receptor (CAR). In some embodiments, the methods include disrupting an area in the subject in which the cells are present or likely to be present and/or a lesion, such as a tumor and/or effecting a treatment that includes one or more of a physical or mechanical manipulation of a lesion or portion thereof, radiation or administration of an immunomodulatory agent. In some embodiments, the disruption and/or treatment alters the environment of the lesion, e.g. tumor microenvironment. In some embodiments, the disruption and/or treatment is a biopsy. In some aspects, the provided methods result in increased expansion, and, in some cases, a more robust and durable response, of the engineered cells after carrying out the disruption and/or treatment.

### BACKGROUND

Various strategies are available for immunotherapy, for example, adoptive cell therapy methods involving administering T cells, such as genetically engineered antigen receptors, such as CARs. In some aspects, available methods may not be entirely satisfactory. There is a need for additional strategies for adoptive cell therapy, e.g., strategies to enhance persistence, activity and/or proliferation of administered cells and responses and strategies for modulating cells. Provided are methods that meet such needs.

### SUMMARY

Provided herein is a method for expanding genetically engineered cells, including effecting disruption of an area in a subject in which the engineered cells are present or likely to be present or were present or were likely to be present and/or effecting a treatment that includes one or more of a physical or mechanical manipulation of a lesion or portion thereof, radiation or administration of an immunomodulatory agent, said subject having previously received administration of genetically engineered cells for treating a disease or condition, wherein the method results in expansion of the engineered cells in the subject, in the area, and/or in a tissue or organ or fluid of the subject and/or in an increased number of the engineered cells in the area, tissue or organ or fluid. In certain embodiments, the area is or comprises a lesion or portion thereof. In some embodiments, the lesion is a tumor. In certain embodiments, the tumor is a primary or secondary tumor. In particular embodiments, the area is or comprises bone marrow tissue. In certain embodiments, area is or comprises a lesion or portion thereof.

In some of any such embodiments, at or immediately prior to the time of the disruption and/or treatment, the subject has relapsed following remission in response to the administration of the genetically engineered cells. In some of any such embodiments, at or immediately prior to the time of the disruption and/or treatment: the subject is in remission; the number of engineered cells detectable in the blood is reduced or is not detectable; the number of engineered cells detectable in a fluid or tissue or sample, optionally the blood, from the subject is decreased compared to a preceding time point after administration of the engineered cells; and/or the number of cells of engineered cells detectable in a fluid or tissue or sample, optionally the blood, from the subject, is decreased by or more than 1.5-fold, 2.0-fold, 3.0-fold, 4.0-fold, 5.0-fold, 10-fold or more as compared to the peak or maximum number engineered cells detectable or detected in the blood of the subject after initiation of administration of the engineered cells and/or compared to the level at a time point within 1, 2, 3, 4, 5, 6, 7, 8, 9, 10, 11, 12, 13, or 14 or 28 days following the administration of the cells.

In some of any such embodiments, the disruption and/or treatment is carried out at, at about, or greater than, or greater than about 2 weeks, 1 month, 2 months, 3 months, 4 months, 5 months, 6 months, 1 year or more after initiation of administration of the genetically engineered cells or after the last dose of the genetically engineered cells. In some of any such embodiments, the disruption and/or treatment comprises one or more of administration of an immunomodulatory agent, radiation or a physical or mechanical manipulation of the area or lesion.

In some of any such embodiments, the disruption and/or treatment comprises administration of an immunomodulatory agent. In particular embodiments, the immunomodulatory agent is or comprises an immune-inhibitory molecule, is or comprises an immune checkpoint molecule or member of an immune checkpoint pathway and/or is or comprises a modulator of an immune checkpoint molecule or pathway. In certain embodiments, the immune checkpoint molecule or pathway is or comprises PD-1, PD-L1, PD-L2, CTLA-4, LAG-3, TIM3, VISTA, an adenosine receptor, CD73, CD39, adenosine 2A Receptor (A2AR), or adenosine or a pathway involving any of the foregoing. In certain embodiments, the immunomodulatory agent is thalidomide or is a derivative or analogue of thalidomide. In particular embodiments, the immunomodulatory agent is lenalidomide or pomalidomide, avadomide, a stereoisomer of lenalidomide, pomalidomide, avadomide or a pharmaceutically acceptable salt, solvate, hydrate, co-crystal, clathrate, or polymorph thereof. In certain embodiments, the immunomodulatory agent is lenalidomide, a stereoisomer of lenalidomide or a pharmaceutically acceptable salt, solvate, hydrate, co-crystal, clathrate, or polymorph thereof.

In some of any such embodiments, after the relapse and prior to the disruption and/or treatment, the subject has not been administered an exogenous or recombinant agent for treating the disease or condition or for modulating the activity of the engineered cells. In some of any such embodiments, the disruption and/or treatment comprises radiation. In some of any such embodiments, the disruption and/or treatment comprises a physical or mechanical manipulation of the area or lesion, optionally comprises probing, poking or penetrating the area or lesion. In some embodiments, the physical or mechanical manipulation comprises a biopsy. In particular embodiments, the biopsy is carried out by a needle or a trocar. In certain embodiments, the biopsy comprises an incisional biopsy.

In some of any such embodiments, the methods results in expansion of the genetically engineered cells or an increase in the number of the genetically engineered cells compared to at the time just prior to the disruption and/or treatment. In particular embodiments, expansion of the cells occurs within or within about 24 hours, 48 hours, 96 hours, 7 days, 14 days or 28 days after the disruption and/or treatment. In certain embodiments, the expansion results in greater than or greater than about 1.5-fold, 2.0-fold, 5.0-fold, 10-fold, 100-fold, 200-fold, or more engineered cells detectable in the blood compared to just prior to the disruption and/or treatment; or the expansion results in greater than or greater than about 1.5-fold, 2.0-fold, 5.0-fold, 10-fold, 100-fold, 200-fold, or more engineered cells detectable in the blood compared to the prior peak levels of engineered cells in the blood prior to the disruption and/or treatment. In particular embodiments, the number of engineered cells detectable in the blood at a time after the disruption and/or treatment is: increased (e.g. increase by 1.5-fold, 2-fold, 3-fold, 4-fold, 5-fold, 10-fold or more decreased) compared to the number of engineered cells at a preceding time point before the disruption and/or treatment; more than 1.5-fold 2-fold, 3-fold, 4-fold, 5-fold, 10-fold, 50-fold, 100-fold or more than the peak or maximum number of engineered cells detectable in the blood of the subject before the disruption and/or treatment; more than or about more than 10%, 9%, 8%, 7%, 6%, 5%, 4%, 3%, 2%, 1%, 0.5%, 0.2% or 0.1% of the engineered cells are detectable in the blood at a time after a peak of maximum level of such cells has been detected in the blood.

In some of any such embodiments, the engineered cells express a recombinant receptor that specifically binds to an antigen associated with the disease or disorder or expressed in cells of the environment or of the lesion. In some of any such embodiments, the disease or condition is a tumor or a cancer. In some of any such embodiments, the disease or condition is a leukemia or lymphoma. In some of any such embodiments, the disease or condition is a non-Hodgkin lymphoma (NHL), an acute lymphoblastic leukemia (ALL) or a chronic lymphocytic leukemia (CLL). In some of any such embodiments, the recombinant receptor is a T cell receptor or a functional non-T cell receptor. In some of any such embodiments, the recombinant receptor is a chimeric antigen receptor (CAR).

In some embodiments, the CAR comprises an extracellular antigen-recognition domain that specifically binds to the antigen and an intracellular signaling domain comprising an ITAM. In certain embodiments, the antigen is CD19. In particular embodiments, the intracellular signaling domain comprises an intracellular domain of a CD3-zeta (CD3) chain. In some embodiments, the CAR further comprises a costimulatory signaling region. In certain embodiments, the costimulatory signaling domain comprises a signaling domain of CD28 or 4-1BB. In particular embodiments, the engineered cells are CD4+ or CD8+ T cells. In some of any such embodiments, the engineered cells are autologous to the subject.

In some of any such embodiments, the engineered cells are allogeneic to the subject. In some of any such embodiments, the engineered cells administered is between about 0.25×10⁶ cells/kg body weight of the subject and 5×10⁶ cells/kg, 0.5×10⁶ cells/kg body weight of the subject and 3×10⁶ cells/kg, between about 0.75×10⁶ cells/kg and 2.5×10⁶ cells/kg or between about 1×10⁶ cells/kg and 2×10⁶ cells/kg, each inclusive. In some of any such embodiments, the engineered cells are administered in a single pharmaceutical composition comprising the cells. In some of any such embodiments, the engineered cells administered is a split dose, wherein the cells of the dose are administered in a plurality of compositions, collectively comprising the cells of the dose, over a period of no more than three days.

Provided herein are a methods of treatment, comprising administering a treatment regimen to a subject, wherein the subject has previously been administered genetically engineered cells for treating a disease or condition, wherein the method results in expansion of the engineered cells in the subject, in the area, and/or in a tissue or organ or fluid of the subject and/or in an increased number of the engineered cells in the area, tissue or organ or fluid.

In certain embodiments the treatment regimen comprises a disruption of an area in a subject in which the engineered cells are present are suspected of being present or having been present, or likely to be present and/or includes one or more of a physical or mechanical manipulation of a lesion or portion thereof, radiation or administration of an immunomodulatory agent. In particular embodiments the treatment regimen and/or the method does not comprise a subsequent administration of genetically engineered cells or of the genetically engineered cells and/or the expansion is achieved without such a subsequent administration. In some embodiments the treatment regimen is administered at a sub-therapeutic dose and/or derives its therapeutic effect via expansion of the genetically engineered cells. In certain embodiments the subject has relapsed after response to, and/or did not respond to, the previous administration of genetically engineered cells. In some embodiments the subject had responded to the genetically engineered cells and has subsequently ceased to respond and/or relapsed.

### BRIEF DESCRIPTION OF THE DRAWINGS

FIG. 1A shows the number of CD3⁺/CAR⁺ T cells in peripheral blood measured at certain time points post-infusion for subjects grouped by best overall response.

FIGS. 1B-1D show CD3⁺/CAR⁺ T cells, CD4⁺/CAR⁺ T, and CD8⁺/CAR⁺ T cell levels in peripheral blood measured at certain time points post-infusion for subjects who achieved a response, grouped by continued response at 3 months.

FIG. 2A shows the number of CD3+/CAR+, CD4+/CAR+, CD8+/CAR+ T cells in peripheral blood of a subject with chemorefractory transformed DLBCL measured at certain time points. FIG. 2B depicts a pretreatment axial PET-CT image showing an intracranial abnormality in the right middle cranial fossa and extensive abnormality in subcutaneous tissues in the right posterior auricular region. FIG. 2C is a post-treatment PET-CT image depicting resolution of the abnormality in FIG. 2B after treatment with anti-CD19 CAR+ T cells. FIG. 2D is a pretreatment brain MRI (high-resolution T₁-weighted image with the use of contrast material; axial view) showing a homogeneously enhancing mass in the right middle cranial fossa. FIG. 2E is a post-treatment MRI image showing near-complete resolution of the enhancing mass. FIG. 2F is an axial PET-CT image at relapse showing right posterior auricular tumor recurrence associated with intense uptake of ¹⁸F-flurodeoxyglycose (arrow). FIG. 2G is a PET-CT imaging showing resolution of the posterior auricular tumor after incisional biopsy and re-expansion of CAR+ T cells.

FIG. 3 shows the percentage of subjects who experienced laboratory abnormalities and treatment-emergent adverse events (TEAEs) that occurred in ≥20% of subjects. *: One Grade 5 AE of multi-organ failure unrelated to study treatment and due to progression of lymphoma; †: One Grade 5 AE of diffuse alveolar damage, investigator assessed as related to fludarabine, cyclophosphamide, and CAR T cell therapy, occurred on day 23 in a subject who refused mechanical ventilation for progressive respiratory failure while neutropenic on growth factors and broad spectrum antibiotics and antifungals

FIG. 4 is a Kaplan meier curve depicting observed time to onset of CRS and neurotoxicity.

FIG. 5A and FIG. 5B depicts response rates among subgroups of treated subjects.

FIG. 6A and FIG. 6B shows the duration of response (CR/PR, CR or PR) and overall survival in the full and core cohort of subjects.

FIG. 7A shows the pharmacokinetics of the CAR⁺ T cells in peripheral blood at various time points post-treatment at different dose levels.

FIG. 7B shows the pharmacokinetics of the CAR⁺ T cells in peripheral blood at various time points post-treatment between responders and nonresponders.

FIG. 7C shows the pharmacokinetics of the CAR⁺ T cells in peripheral blood at various time points post-treatment in subjects that did or did not develop any neurotoxicity.

FIG. 8 shows levels of analytes measured in the serum of subjects prior to administration of the CAR+ T cells and correlation to the development of neurotoxicity.

FIG. 9 shows a graph plotting progression-free time (months) and indicating best overall response and response durability, and individual clinical outcomes observed over time in individual subjects within a Full cohort and a Core cohort of NHL subjects treated with an anti-CD19 cell therapy containing CAR-T-expressing CD4+ and CD8+ T cells. ^{a}: Patients achieved BOR at month 1 except where otherwise noted; ^{b}: Complete resolution of CNS involvement by lymphoma observed in 2 patients; ^{c}: One patient re-expanded after biopsy upon disease progression

### DETAILED DESCRIPTION

### I. MODULATING GENETICALLY ENGINEERED CELLS IN ADOPTIVE CELL THERAPY

Provided herein are methods for modulating genetically engineered cells in vivo, such as boosting, augmenting or increasing the expansion, proliferation and/or activation of genetically engineered cells administered to a subject. In some embodiments, after administering genetically engineered cells, such as recombinant receptor-expressing cells (e.g. CAR+ T cells) to a subject, the provided methods involve disrupting, such as manipulating, an area in the subject, such as a tissue, organ, mass or lesion area of the subject or a region or portion thereof, in which the engineered cells are present or likely to be present and/or effecting a treatment that includes one or more of a physical or mechanical manipulation of a lesion or portion thereof, radiation or administration of an immunomodulatory agent. In some embodiments, the area (e.g. a tissue, organ, mass or lesion areas of the subject or a region or portion thereof) is known or suspected of containing antigen-expressing cells recognized by the genetically engineered cells. In particular, relative to other areas or regions in the subject, the targeted area is one in which the area is known or suspected to have a higher or greater concentration or amount of antigen or number of antigen-specific cells relative to or compared to other areas in the subject.

In some embodiments, the treatment and/or disruption alters the environment of the area, such as alters the environment of the lesion, e.g. tumor microenvironment. In some cases, the alteration is such to directly or indirectly modulate activity of the genetically engineered T cells. In some aspects, the alteration is sufficient to promote the in vivo re-activation, expansion and/or proliferation of the previously administered genetically engineered cells, such as recombinant receptor-expressing cells (e.g. CAR+ T cells).

T cell-based therapies, such as adoptive T cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of cancer and other diseases and disorders. In certain contexts, available approaches to adoptive cell therapy may not always be entirely satisfactory. In some contexts, optimal efficacy can depend on the ability of the administered cells to recognize and bind to a target, e.g., target antigen, to traffic, localize to and successfully enter appropriate sites within the subject, tumors, and environments thereof. In some contexts, optimal efficacy can depend on the ability of the administered cells to become activated, expand, to exert various effector functions, including cytotoxic killing and secretion of various factors such as cytokines, to persist, including long-term, to differentiate, transition or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and avoid or reduce exhaustion, anergy, peripheral tolerance, terminal differentiation, and/or differentiation into a suppressive state.

In some aspects, the efficacy of the immunotherapy, e.g., T cell therapy, may be limited by the immunosuppressive activity or factors present in the local microenvironment of the disease or disorder, e.g., the TME. In some aspects, the TME contains or produces factors or conditions that can suppress the activity, function, proliferation, survival and/or persistence of T cells administered for T cell therapy.

In some embodiments, the exposure and persistence of engineered cells is reduced or declines after administration to the subject. Yet, observations indicate that, in some cases, increased exposure of the subject to administered cells expressing the recombinant receptors (e.g., increased number of cells or duration over time) may improve efficacy and therapeutic outcomes in adoptive cell therapy. Preliminary analysis conducted following the administration of different CD19-targeting CAR-expressing T cells to subjects with various CD19-expressing cancers in multiple clinical trials revealed a correlation between greater and/or longer degree of exposure to the CAR-expressing cells and treatment outcomes. Such outcomes included patient survival and remission, even in individuals with severe or significant tumor burden. In some aspects, the safety profile observed by the provided methods may reduce risks of unwanted safety concerns of a combination therapy involving a therapeutic T cell composition as provided and another therapy for treating the disease or condition, e.g. an immunomodulatory agent, such as a checkpoint antagonist.

It is found herein that disrupting a lesion and/or effecting a treatment that includes one or more of a physical or mechanical manipulation of a lesion or portion thereof, radiation or administration of an immunomodulatory agent, in a subject having been administered genetically engineered T cells, results can result in substantial expansion of the cells in the subject, even after the subject has relapsed. Provided herein are methods of disrupting, such as manipulating, an area in a subject in which cells are or are likely to be present and/or effecting a treatment that includes one or more of a physical or mechanical manipulation of a lesion or portion thereof, radiation or administration of an immunomodulatory agent. In some embodiments, the area is a lesion, such as a tumor or a cancer. In some embodiments, the disruption and/or treament can be carried out by a mechanical or physical alteration at or near the lesion, e.g. at or near tumor, or at or near a microenvironment that is associated with the lesion, e.g. a tumor microenvironment (TME). In some embodiments, the disruption and/or treatment can be effected by administration of a pharmacologic agent or therapeutic agent, such as an immunomodulatory agent or other agent capable of modulating activity of the T cells or of a cell or cells associated with the lesion or a microenvironment of the lesion. In some cases, the pharmacologic agent is a therapeutic agent targeted to the site of the lesion or that specifically binds to a cell of the lesion, e.g. to a cell of the tumor microenvironment. In some embodiments, the disruption and/or treatment, such as by mechanical disruption or by administration of a pharmacologic agent, such as an immunomodulatory agent, is carried out greater than or greater than about one week, two months, one month, two months, three months, four months, five months, six months, 1 year, 2 years or more after initiation of administration of the recombinant receptor-expressing T cells, e.g. CAR+ T cells.

In some embodiments, at or immediately prior to the time of the disruption and/or treatment, such as administration of the pharmacologic agent or therapeutic agent, the subject has relapsed following remission after treatment.

A. Lesions

In aspects of the provided methods, the area in a subject in which the engineered cells are present or likely to be present or were present or were likely to be present is a lesion or portion of a lesion. In some aspects, the lesion is one that is known or suspected of containing antigen-expressing cells recognized by the administered recombinant receptor-expressing cells. The provided methods are carried out to disrupt the lesion and/or to treat the subject to modulate the genetically engineered cells in vivo.

In some embodiments, a lesion includes any region of an organ or tissue that has suffered damage through injury or a disease. In certain embodiments, a lesion is any region of an organ or tissue that has undergone and/or is undergoing an abnormal change in structure due to an injury or disease. In some embodiments, the lesion is circumscribed and well-defined. In some embodiments, the lesion is non-cancerous. In particular embodiments, the lesion is non-tumorous. In certain embodiments, the lesion is cancerous or is suspected of being cancerous. In particular embodiments, the lesion is a tumor.

In some embodiments, the lesion is a lesion found on an organ or tissue. In certain embodiments, a lesion is present in connective tissue, muscle tissue, nervous tissue, or epithelial tissue. In certain embodiments, the lesion is present on the heart, vasculature, salivary glands, esophagus, stomach, liver, gallbladder, pancreas, intestines, colon, rectum, hypothalamus, pituitary gland, pineal gland, thyroid, parathyroid, adrenal gland, kidney, ureter, bladder, urethra, lymphatic system, skin, muscle, brain, spinal cord, nerves, ovaries, uterus, testes, prostate, pharynx, larynx, trachea, bronchi, lungs, diaphragm, bone, cartilage, ligaments, or tendons.

In certain embodiments, the lesion is selected from: a lesion in the soft tissue, e.g., a Morel-Lavallee lesion, a Bankart lesion, a Perthes lesion, a Stener lesion, or a SLAP lesion; a bone lesion, e.g., a nonossifying fibroma, a ALPSA lesion, or a Hill-Sachs lesion; a skin lesion, e.g., a melanocytic nevus, a skip lesion, or an Osler's node; keratoderma blennorrhagicum, dermatosis papulosa nigra, a leukemid, a Janeway lesion, Kaposi's sarcoma, Nevus spilus, or chronic scar keratosis; a gastrointestinal lesion, e.g., Dieulafoy's lesion or a Cameron lesion; an endodermal lesion, e.g., a melanocytic oral lesion, endometrial intraepithelial neoplasia; another lesion such as a Ghon focus, a benign lymphoepithelial lesion, a multiple sclerosis lesion, a tropical ulcer, or herpetic whitlow.

In certain embodiments, the lesion is a tumor or a neoplasm. In certain embodiments, the tumor is benign. In particular embodiments, the tumor is precancerous or cancerous, or is suspected of being cancerous or precancerous. In certain embodiments, the tumor is a primary tumor, i.e., the tumor is found at the anatomical site where the lesion initially developed or appeared. In some embodiments, the tumor is a secondary tumor, e.g., a cancerous tumor that originated from a cell within a primary tumor located within a different site in the body.

In some embodiments, a lesion is associated with or caused by, or is suspected of being associated with or caused by, a cancer or proliferative disease that is a B cell malignancy or hematological malignancy. In some embodiments, the cancer or proliferative disease is lymphoblastic leukemia (ALL), non-Hodgkin's lymphoma (NHL), or chronic lymphocytic leukemia (CLL). In some embodiments, the cancer is CLL. In some embodiments, the lesion is associated with or caused by, or suspected of being associated with or caused by, a myeloma, a lymphoma or a leukemia. In some embodiments, the lesion is associated with or caused by, or suspected of being associated with or caused by, a non-Hodgkin lymphoma (NHL), an acute lymphoblastic leukemia (ALL), a chronic lymphocytic leukemia (CLL), a diffuse large B-cell lymphoma (DLBCL), acute myeloid leukemia (AML), or a myeloma, e.g., a multiple myeloma (MM). In some embodiments, the lesion is associated with or caused by, or suspected of being associated with or caused by, a MM or a DBCBL.

In particular embodiments, the lesion is associated with or caused by, or is suspected of being associated with or caused by, a non-hematologic cancer, e.g., the lesion is a solid tumor. In some embodiments, the lesion is associated with or caused by, or is suspected of being associated with or caused by, a bladder, a lung, a brain, a melanoma (e.g. small-cell lung, melanoma), a breast, a cervical, an ovarian, a colorectal, a pancreatic, an endometrial, an esophageal, a kidney, a liver, a prostate, a skin, a thyroid, or a uterine cancer. In some embodiments, the lesion is associated with or caused by a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma.

In particular embodiments, the lesion is a tumor that contains, or is suspected of containing, at least one cancer cell. In some embodiments, the lesion is a tumor that contains, or is suspected of containing, a cancer cell derived from a(n) AIDS-related cancer, a breast cancer, a cancer of the digestive/gastrointestinal tract, an anal cancer, an appendix cancer, a bile duct cancer, a colon cancer, a colorectal cancer, an esophageal cancer, a gallbladder cancer, islet cell tumors, pancreatic neuroendocrine tumors, a liver cancer, a pancreatic cancer, a rectal cancer, a small intestine cancer, a stomach (gastric) cancer, an endocrine system cancer, an adrenocortical carcinoma, a parathyroid cancer, a pheochromocytoma, a pituitary tumor, a thyroid cancer, an eye cancer, an intraocular melanoma, a retinoblastoma, a bladder cancer, a kidney (renal cell) cancer, a penile cancer, a prostate cancer, a transitional cell renal pelvis and ureter cancer, a testicular cancer, a urethral cancer, a Wilms' tumor or other childhood kidney tumor, a germ cell cancer, a central nervous system cancer, an extracranial germ cell tumor, an extragonadal germ cell tumor, an ovarian germ cell tumor, a gynecologic cancer, a cervical cancer, an endometrial cancer, a gestational trophoblastic tumor, an ovarian epithelial cancer, a uterine sarcoma, a vaginal cancer, a vulvar cancer, a head and neck cancer, a hypopharyngeal cancer, a laryngeal cancer, a lip and oral cavity cancer, a metastatic squamous neck cancer, a nasopharyngeal cancer, an oropharyngeal cancer, a paranasal sinus and nasal cavity cancer, a pharyngeal cancer, a salivary gland cancer, a throat cancer, a musculoskeletal cancer, a bone cancer, a Ewing's sarcoma, a gastrointestinal stromal tumors (GIST), an osteosarcoma, a malignant fibrous histiocytoma of bone, a rhabdomyosarcoma, a soft tissue sarcoma, a uterine sarcoma, a neurologic cancer, a brain tumor, an astrocytoma, a brain stem glioma, a central nervous system atypical teratoid/rhabdoid tumor, a central nervous system embryonal tumors, a central nervous system germ cell tumor, a craniopharyngioma, an ependymoma, a medulloblastoma, a spinal cord tumor, a supratentorial primitive neuroectodermal tumors and pineoblastoma, a neuroblastoma, a respiratory cancer, a thoracic cancer, a non-small cell a lung cancer, a small cell lung cancer, a malignant mesothelioma, a thymoma, a thymic carcinoma, a skin cancer, a Kaposi's sarcoma, a melanoma, or a Merkel cell carcinoma.

B. Treating and/or Disrupting a Lesion

Provided herein are methods for effecting treatment and/or disrupting a lesion, e.g. a tumor, to modulate genetically engineered cells in vivo, e.g., boosting, augmenting or increasing the expansion of genetically engineered cells administered to a subject. In some embodiments, the treatment and/or disruption includes mechanical disruption, e.g., a biopsy, treatment and/or disruption by irradiation, e.g., external beam radiation, and/or pharmacological disruption, e.g., treatment with an immunomodulatory agent.

In some embodiments, a treatment and/or a disruption of a lesion is any manipulation, procedure, or treatment that alters the engineered T cells, such as recombinant receptor-expressing T cells, e.g. CAR+ T cells, directly or indirectly, such as by altering the microenvironment associated with the lesion. In some embodiments, the manipulation, procedure, or treatment is a mechanical disruption, e.g. a biopsy. In particular embodiments, the manipulation, procedure, or treatment is an administration of a pharmacological agent to the subject with the lesion. In particular embodiments, the manipulation, procedure, or treatment is an application of radiation to the lesion. In some embodiments, the treatment and/or disruption, at least initially or immediately, reduces the number of cells in the lesion.

In certain embodiments, the treatment and/or disruption of a lesion comprises disrupting of an area in a subject in which engineered cells, e.g., cells expressing a CAR, are present or likely to be present. In some embodiments, disrupting a lesion comprises disrupting an area where the genetically engineered cells were once present or an area where genetically engineered cells were likely to have present.

In particular embodiments, a lesion is treated and/or disrupted to modulate genetically engineered cells in vivo, wherein the treatment and/or disruption is or results in an alteration of the lesion or a microenvironment that is associated with the lesion, e.g. a tumor microenvironment (TME). In some embodiments, the alteration may include a modification, change, permutation, or transformation of at least one component of the microenvironment. In certain embodiments, the alteration refers to a modification, change, permutation, or transformation of the lesion or microenvironment as compared to the lesion or microenvironment before the treatment and/or disruption was performed. In particular embodiments, the alteration refers to a modification, change, permutation, or transformation of the lesion or microenvironment as compared to a similar lesion, e.g., a lesion of the same type, e.g. tumor type, or microenvironment of the same tumor type, that did not receive a treatment and/or disruption. In certain embodiments, the similar lesion is in a different subject. In particular embodiments, the similar lesion is in the same subject as the treated and/or disrupted lesion.

In some embodiments, the components of the microenvironment comprise cells within or surrounding the lesion, e.g., cancer cells, non-lesion cells, and molecules, e.g., signaling molecules, that are secreted, released, and/or expressed by the cells within the microenvironment. Non-lesion cells may include cells that are not cells of the lesion that but are contained at the periphery or within the lesion. Non-lesion cells may include, but are not limited to, immune cells, fibroblasts, adipocytes, a vascular endothelial cells, pericytes, and lymphatic endothelial cells. Signaling molecules may include, but are not limited to, cytokines, chemokines, growth factors, and inflammatory and matrix remodeling enzymes.

In particular embodiments, the treatment and/or disruption is a manipulation, procedure, or treatment that reduces, at least initially or for a period of time, the number of cells, or the number of cells of at least one cell type, within the microenvironment of the lesion. In certain embodiments, the lesion is a tumor, and the manipulation, procedure, or treatment reduces the number of tumor cells in the lesion. In particular embodiments, the lesion is cancerous, and the manipulation, procedure, or treatment reduces the number of cancer cells in the lesion. In some embodiments, the treatment and/or disruption is a manipulation, procedure, or treatment that decreases the number of non-lesion cells that are within the microenvironment of the lesion. Non-lesion cells found within the microenvironment of the lesion include, but are not limited to, immune cells, fibroblasts, adipocytes, a vascular endothelial cells, pericytes, and/or lymphatic endothelial cells that are within the microenvironment. In certain embodiments, the treatment and/or disruption is a manipulation, procedure, or treatment that results in an increase in the number of immune cells, fibroblasts, adipocytes, a vascular endothelial cells, pericytes, and/or lymphatic endothelial within the microenvironment of the tumor.

In particular embodiments, the treatment and/or disruption is a manipulation, procedure, or treatment that removes or kills cells of the lesion, at least initially or for a period of time. In some embodiments, the lesion is tumor, and the manipulation, procedure, or treatment kills or removes tumor cells in the lesion, at least initially or for a period of time. In particular embodiments, the lesion is cancerous, and the manipulation, procedure, or treatment kills or removes cancer cells in the lesion. In some embodiments, the treatment and/or disruption kills or removes at least about 0.001%, at least about 0.01%, at least about 0.1%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 99.9% of the cells of the lesion.

In particular embodiments, the treatment and/or disruption is a manipulation, procedure, or treatment that results in an alteration in the number of one or more types of immune cells in the microenvironment of the lesion. The types of immune cells that are found in a microenvironment may include, but are not limited to, T lymphocytes, B lymphocytes, natural killer cells (NK cells), natural killer T cell (NKT cells), macrophages, e.g., tumor associated macrophages, myeloid-derived suppressor cells (MDSC), dendritic cells, and neutrophils, e.g., a tumor associated neutrophils (TANs). In certain embodiments, the treatment and/or disruption is a manipulation, procedure, or treatment that increases the number of CD8⁺ cells, cytotoxic memory CD8⁺ T cells (CD8⁺CD45RO⁺), CD4⁺ T helper 1 (T_{H}1) cells, CD4⁺ T helper 2 (T_{H}2) cells, natural killer (NK) cells, natural killer T (NKT) cells, and/or γδT lymphocytes in the microenvironment of the lesion. In particular embodiments, the treatment and/or disruption is a manipulation, procedure, or treatment that decreases the number of TH2 cells, CD4⁺ T helper 17 (T_{H}17) cells, immunosuppressive T regulatory cells (Tregs), regulatory B cells (Bregs), B10 cells, and/or Tumor-associated macrophages (TAMs) in the microenvironment of the lesion.

In some embodiments, the treatment and/or disruption is a manipulation, procedure, or treatment that increases the number of one or more signaling molecules that are present in the lesion and/or the microenvironment of the lesion. In some embodiments, signaling molecules may include, but are not limited to, cytokines, chemokines, growth factors, and inflammatory and matrix remodeling enzymes. In certain embodiments, manipulation, procedure, or treatment increases an amount of interleukin-2 (IL-2), interleukin-17A (IL-17A), interleukin-17F (IL-17F), interleukin-21 (IL-21), interleukin 22 (IL-22), and/or interferon gamma (IFN-γ). In some embodiments, the treatment and/or disruption is a manipulation, procedure, or treatment that increases an amount of a signaling molecule that is present in the lesion and/or in the microenvironment of the lesion by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 1-fold, at least about 1.5 fold, at least about 2-fold, at least about 2.5-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40 fold, at least about 50-fold, at least about 100-fold, at least about 200-fold, at least about 500-fold, or at least about 1,000 fold.

In some embodiments, the treatment and/or disruption is a manipulation, procedure, or treatment that decreases the number of one or more signaling molecules that are present in the lesion and/or the microenvironment of the lesion. In certain embodiments, manipulation, procedure, or treatment decreases an amount of interleukin-4 (IL-4), interleukin-5 (IL-5), interleukin-10 (IL-10), interleukin-13 (IL-13), interleukin-17A (IL-17A), interleukin-17F (IL-17F), interleukin-21 (IL-21), interleukin 22 (IL-22), transforming growth factor beta (TGF-β), vascular endothelial growth factor (VEGF), endothelin-1, endothelin-2, endothelin-3, endothelial-monocyte-activating polypeptide II (EMAP2, also known as AIMP1), a hepatocyte growth factor (HGF), a fibroblast growth factor (FGF), insulin-like growth factor 1 (IGF1), insulin-like growth factor 2 (IGF2), TGF-β, —C—X—C motif chemokine 12 (CXCL12), a platelet-derived growth factor (PDGF), a matrix metalloprotease (MMP) and/or a cathepsins, e.g., Cathepsin L. In some embodiments, the treatment and/or disruption is a manipulation, procedure, or treatment that decreases the amount of the signaling molecule in the lesion and/or the microenvironment of the lesion by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 99.9%, or about 100%.

1. Mechanical Disruption

In some embodiments, the treatment and/or disruption involves the physical or mechanical manipulation of the area, such as of the lesion, e.g. by probing, poking and/or penetrating the lesion. In some embodiments, treatment and/or disruption is carried out by a biopsy of the area, such as a biopsy of the lesion (e.g. tumor). In some embodiments, biopsy is carried out with a needle. In some embodiments, the biopsy is an incisional biopsy.

In some embodiments, the lesion is physically and or mechanically disrupted with a biopsy procedure to modulate genetically engineered cells in vivo, e.g., to boost, augment, or increase the expansion of genetically engineered cells administered to a subject. In some embodiments, the biopsy procedure is a fine needle aspiration, whereby a long, thin needle that can be inserted into the lesion and a syringe is used to draw out cells and/or fluid from the lesion. In particular embodiments, the biopsy is a core needle biopsy, whereby a larger needle with a cutting tip used to remove a column of tissue from the lesion. In particular embodiments, the biopsy is a vacuum-assisted biopsy, whereby a suction device is used to increase the amount of fluid and/or cells that are extracted through a needle. In certain embodiments, the biopsy is an image-guided biopsy, whereby the lesion is visualized with imaging techniques, including, but not limited to, X-ray, ultrasound, CT scanning, or MRI scanning to allow for a health care provider, e.g., a doctor, to visualize the lesion and to guide a biopsy instrument, e.g., a needle, to the tumor.

In particular embodiments, a lesion is disrupted with one or more biopsy instruments (e.g., a needle) to modulate genetically engineered cells in vitro. In some embodiments, the biopsy instrument is a core needle. In certain embodiments, the biopsy instrument is a needle that may be used for fine-needle aspiration. In certain embodiments, the biopsy instrument is a trocar.

In particular embodiments, the biopsy instrument is a core needle. In some embodiments, the core needle is 10 gauge, 11 gauge, 12 gauge, 13 gauge, 14 gauge, 15 gauge, 16 gauge, 17 gauge, 18 gauge, 19 gauge, 20 gauge, 21 gauge, 22 gauge, 23 gauge, 24 gauge, 25 gauge, or 26 gauge. In certain embodiments, the core needle is between 10 gauge and 30 gauge, between 10 gauge and 24 gauge, or between 14 gauge and 20 gauge. In certain embodiments, the needle is about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, or about 32 cm in length. In some embodiments, the core needle is between about 5 cm and about 30 cm, between about 10 cm and about 25 cm, or between about 10 cm and about 20 cm in length. In certain embodiments, the core needle is between 14 gauge and 20 gauge and is between about 10 cm and 20 cm in length. In particular embodiments, the core needle is disposable. In certain embodiments, the core needle is reusable.

In some embodiments, the lesion is disrupted by fine-needle aspiration. In particular embodiments, the needle is a fine-needle. In some embodiments, the needle may be used for fine-needle aspiration is 20 gauge, 21 gauge, 22 gauge, 23 gauge, 24 gauge, 25 gauge, 26 gauge, 27 gauge, 28 gauge, 29 gauge, 30 gauge, 31 gauge, or 32 gauge. In certain embodiments, the needle is between 20 gauge and 30 gauge, between 22 gauge and 28 gauge, between 20 gauge and 26 gauge, or between about 24 gauge and about 28 gauge. In certain embodiments, the needle may be used for fine-needle aspiration and is about 1 cm, about 2 cm, about 3 cm, about 4 cm, about 5 cm, about 6 cm, about 7 cm, about 8 cm, about 9 cm, about 10 cm, about 12 cm, about 14 cm, about 16 cm, about 18 cm, or about 20 cm in length. In some embodiments, the needle may be used for fine needle aspiration and is between about 1 cm and about 10 cm, between about 5 cm and about 10 cm, or between about 1 cm and about 5 cm. In certain embodiments, the needle may be used for fine-needle aspiration and is between 22 gauge and 28 gauge and between 1 cm and 10 cm in length.

In some embodiments, the lesion is disrupted with a trocar or with the aid of a trocar to modulate genetically engineered cells in vivo. Trocars are commonly used for laparoscopy surgical techniques, for example to gain and secure access to a body cavity, e.g. peritoneal cavity. A conventional trocar may include, for example, a seal, a sharp trocar, a cannula, and a safety shield to protect organs once the trocar has penetrated the abdominal wall. The safety shield is generally designed as a mechanical device which is spring-loaded and activated when the trocar tip is inserted into the cannula. The tip of the trocar is protected by the safety shield. As the trocar passes through the layers of the abdominal wall, the safety shield is retracted, exposing the sharp tip of the trocar. When the device finally penetrates the last layer of abdominal tissue, and just prior to entering the open space of the abdomen, the safety shield moves forward to again cover the trocar tip.

Examples of trocars include bladed trocars, which comprise a bladed tip, and blunt trocars. The type of bladed trocar tip that has been used most commonly is the three-sided pyramidal design, which facilitates entry into a tissue by way of the three sharp edges that can slice through tissue e.g. tissue of the abdominal wall. Bladed trocars also include trocars with hybrid tips. The hybrid tips have smaller leading linear blades to create incisions that are then dilated by a blunt component of the trocar. Blunt trocars that are designed to enter the cavity without a bladed tip. Blunt trocars include radially dilating trocars, which are designed to enter a tissue once a small incision has been made with a different instrument, e.g., a scalpel. In some embodiments, the lesion is disrupted with, or with the aid of, a bladed trocar. In certain embodiments, the lesion is disrupted with, or with the aid of, a blunt trocar.

In some embodiments, the lesion is disrupted with a trocar that is at least or about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm in diameter. In certain embodiments, the trocar has a diameter of between about 1 mm and about 20 mm, between about 1 mm and about 15 mm, between about 5 mm and about 15 mm, between about 10 mm and about 15 mm, or between about 5 mm and about 12 mm. In certain embodiments, the trocar has a diameter of between about 5 mm and about 12 mm.

In certain embodiments, the lesion is disrupted with a punch biopsy to modulate engineered cells in vivo. In some embodiments, a punch biopsy is performed with a circular blade that can be rotated down through the tissue to collect a cylindrical core tissue sample, e.g., a skin sample. In certain embodiments, the punch has a diameter of at least or about 0.1 mm, about 0.5 mm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm in diameter. In certain embodiments, the punch has a diameter of between about 1 mm and about 8 mm.

In some embodiments, the lesion is disrupted with an excisional biopsy. In certain embodiments, an excisional biopsy comprises removal of all or most of the lesion. In particular embodiments, an excisional biopsy comprises the removal of at least about 90%, at least about 95%, at least about 98%, at least about 99%, at least about 99.5%, at least about 99.9%, or about 100% of the lesion.

In particular embodiments, the lesion is disrupted with an incisional biopsy. In certain embodiments, an incisional biopsy comprises removal of at least a portion of the lesion. In certain embodiments, the incisional biopsy comprises removal of at least about 0.01%, at least about 0.1%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the lesion. In particular embodiments, the incisional biopsy comprises the removal of less than about 50%, less than about 40%, less than about 30%, less than about 20%, less than about 10%, less than about 5%, less than about 1%, less than about 0.5%, or less than about 0.1% of the lesion.

In some embodiments, the lesion is disrupted by poking, prodding, cutting, lacerating, cleaving, opening, nicking, shaving, and/or sectioning the lesion with a surgical tool, e.g., a trocar, a knife, or a needle. In particular embodiments, the poking, prodding, cutting, lacerating, cleaving, opening, nicking, shaving, and/or sectioning of the lesion results in an injury to the lesion. For example, in some embodiments, the injury comprises a puncture, a piercing, a slice, a slit, or a tear. In some embodiments, disrupting the lesion results in an injury to the lesion that comprises a puncture, a piercing, a slice, a slit, or a tear that is less than or about 0.001 mm, about 0.01 mm, about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.5 mm, about 3 mm, about 4 mm, about 5 mm, about 6 mm, about 7 mm, about 8 mm, about 9 mm, about 1 cm, about 2 cm, about 3 cm, about 4 cm, about 5 cm, about 6 cm, about 7 cm, about 8 cm, about 9 cm, or about 10 cm. In some embodiments, the puncture, piercing, slice, slit, or tear is great than or equal to about 10 cm. In certain embodiments, the lesion is disrupted by poking, prodding, cutting, lacerating, cleaving, opening, nicking, shaving, and/or sectioning the lesion with a surgical tool, resulting in a puncture, piercing, slice, slit, or tear to the lesion that is between about 0.001 mm and about 0.1 mm, between about 0.1 mm and about 1 mm, between about 1 mm and about 1 cm, or between about 1 cm and about 10 cm.

In some embodiments, the mechanical disruption is performed by altering the temperature of the lesion, e.g., a thermotherapy. In particular embodiments, the mechanical disruption is a cryoablation therapy. In certain embodiments, the mechanical disruption is a hyperthermic therapy.

In certain embodiments, the lesion is disrupted with cryoablation therapy. Cryoablation therapy involves freezing of a neoplastic mass, leading to deposition of intra- and extra-cellular ice crystals; disruption of cellular membranes, proteins, and organelles; and induction of a hyperosmotic environment, thereby causing cell death. Methods for and apparatuses useful in cryoablation therapy are described in Murphy et al, Sent. Urol. Oncol. 79:133-140 (2001) and U.S. Pat. Nos. 6,383,181, 6,383,180, 5,993,444, 5,654,279, 5,437,673, and 5,147,355.

In particular embodiments, the lesion is disrupted with hyperthermic therapy. Hyperthermic therapy typically involves elevating the temperature of a neoplastic mass to a range from about 42° C. to about 44° C. The temperature of the lesion may be further elevated above this range; however, such temperatures can increase injury to surrounding healthy tissue while not causing increased cell death within the lesion to be treated. The tumor may be heated in hyperthermic therapy by any means known to one of skill in the art. In some embodiments, the lesion may be heated by microwaves, high intensity focused ultrasound, ferromagnetic thermoseeds, localized current fields, infrared radiation, wet or dry radiofrequency ablation, laser photocoagulation, laser interstitial thermic therapy, and electrocautery. Microwaves and radio waves can be generated by waveguide applicators, horn, spiral, current sheet, and compact applicators.

Other methods, apparatuses, and compositions for raising the temperature of a lesion, e.g., a tumor, are reviewed in Wust et al, Lancet Oncol. 3:487-97 (2002), and described in U.S. Pat. Nos. 6,470,217, 6,379,347, 6,165,440, 6,163,726, 6,099,554, 6,009,351 , 5,776,175, 5,707,401, 5,658,234, 5,620,479, 5,549,639, and 5,523,058.

2 Treatment and/or Disruption by Irradiation

In certain embodiments, a lesion is treated and/or disrupted by treatment with irradiation, or radiation therapy, to modulate genetically engineered cells in vivo. In certain embodiments, radiation therapy uses high-energy radiation to shrink a lesion, e.g., a tumor, and kill cells within the lesion, e.g., cancer cells. In some embodiments, the lesion is treated and/or disrupted with ionizing radiation, i.e., radiation comprising particles or photons having sufficient energy or can produce sufficient energy via nuclear interactions to produce ionization (gain or loss of electrons). In some embodiments, the lesion is treated and/or disrupted by exposing the lesion to X-rays, gamma rays, charged particles, e.g., electrons, or any other type of radiation that may be used for cancer treatment.

Radiation therapy may include any sources of therapeutic radiation used for the treatment of cancer and/or related disease including, but not limited to, ionizing radiation therapy, brachytherapy, sealed source radiation therapy, systemic radioisotope therapy, unsealed source radiotherapy, radionuclide therapy, external beam radiation therapy, radiation surgery, charged-particle radiotherapy, neutron radiotherapy, x-ray therapy, gamma-ray therapy, and cobalt therapy.

In certain embodiments, the lesion is treated and/or disrupted with external beam therapy (EBT), in which an external source of ionizing radiation is applied to subject at the region of the subject's body that contains the lesion. In some embodiments, EBT comprises orthovoltage (i.e., superficial) beams of radiation to treat and/or disrupt a lesion present on the skin. In certain embodiments, EBT comprises megavoltage, e.g., deep, beams of radiation are used to treat internal lesions, e.g., lesions of the bladder, bowel, prostate, lung, or brain. In particular embodiments, treat and/or disrupting the lesion with EBT comprises delivering X ray rays, gamma rays, electron beams, proton beams, or beams of ionized nuclei to the lesion. In some embodiments, the lesion is treated and/or disrupted by EBT that is performed with a linear accelerator, a collimator, a cobalt machine, a superficial radiation therapy (SRT) machine, Orthovoltage X ray machine.

In some embodiments, the lesion is treated and/or disrupted with internal radiation therapy, i.e., brachytherapy. In certain embodiments, the brachytherapy comprises applying sources of radiation at or near the area of the lesion. In particular embodiments, the brachytherapy comprises interstitial radiation wherein the radiation source is contained in small pellets, seeds, wires, tubes, and/or containers and is placed directly into or next to the lesion. In certain embodiments, the brachytherapy comprises intracavitary radiation, wherein a container of radioactive material is placed in a cavity of the body, e.g., chest cavity or large intestine. In some embodiments, ultrasounds, X-rays, and/or CT scans are used to assist with the placement of the radioactive source.

In some embodiments, the lesion is treated and/or disrupted with permanent brachytherapy, which comprises placing small containers, e.g., containers approximately the size of a grain of rice, into a lesion. In some embodiments, containers give off radiation for a time period of several weeks or months, and are left in place after the radiation is used up.

In particular embodiments, the lesion is treated and/or disrupted with temporary brachytherapy which comprises placing cylinders, hollow needles, tubes (catheters), and/or fluid-filled balloons into the area to be treated that are then removed after treatment. In some embodiments, radioactive materials are placed in these containers for a short time and then removed. In some embodiments, the temporary brachytherapy can be high-dose rate (HDR) brachytherapy, wherein the radiation source is put into place for a few minutes at a time at or near the lesion, and then is removed. This process may be repeated twice a day for up to a week, or once a week for a few weeks. In some embodiments, the temporary brachytherapy is low dose rate (LDR) brachytherapy, wherein the radiation source stays in place for up to 7 days before it is removed.

In some embodiments, the lesion is treated and/or disrupted by systemic radiation therapy. In some embodiments, the systemic radiation therapy comprises administering radioactive substances, such as radioactive iodine, that travel through the blood to kill cells of lesion. Representative radioisotopes that can be administered in radionuclide therapy include, but are not limited to, phosphorus-32, yttrium-90, dysprosium-165, indium-111, strontium-89, samarium-153, rhenium-186, iodine-131, iodine-125, lutetium-177, and bismuth-213. While all of these radioisotopes may be linked to a biomolecule providing specificity of targeting, iodine-131, indium-111, phosphorus-32, samarium-153, and rhenium-186 may be administered systemically without such conjugation. One of skill in the art may select a specific biomolecule for use in targeting a particular neoplasm for radionuclide therapy based upon the cell-surface molecules present on that neoplasm. In some embodiments, a radioactive particle is conjugated to a monoclonal antibody, or active fragment or variant thereof, that binds to a cell of the lesion. Examples of radiopharmaceutical drugs include, but are not limited to, Ibritumomab tiuxetan, an anti-CD20 monoclonal antibody that is conjugated to either yttrium-90 or indium-111; and tositumomab, an anti-CD20 monoclonal antibody that is conjugated to iodine-131.

3. Pharmacological Treatment and/or Disruption

In some embodiments, a lesion is treated and/or disrupted to modulate genetically engineered cells in vivo, e.g., to boost, augment, or increase the expansion of genetically engineered cells administered to a subject, by administering an agent to the subject. In particular embodiments, the agent in a pharmaceutical agent. In some embodiments, the agent is a therapeutic agent. In some embodiments, the agent is an immunomodulatory agent. In particular embodiments, the agent is a chemotherapeutic agent.

In some embodiments, the agent, such as an immunomodulatory agent, is capable of inhibiting or blocking a function of a molecule, or signaling pathway involving said molecule. In some embodiments, the molecule is expressed on an immune cells or is part of an immune synapse, such as is expressed on a T cell or antigen presenting cell or other cell associated with an immune response. In some such aspects, the molecule is an immune-inhibitory molecule or the molecule is an immune checkpoint molecule. In some embodiments, the immune checkpoint molecule or pathway is PD-1, PD-L1, PD-L2, CTLA-4, LAG-3, TIM3, VISTA, adenosine 2A Receptor (A2AR), or adenosine or a pathway involving any of the foregoing.

In some embodiments, the chemotherapeutic agent is or comprises an antibody, which can be an antibody fragment, a single-chain antibody, a multispecific antibody, or an immunoconjugate. In some embodiments, the antibody specifically binds to the immune checkpoint molecule or a ligand or receptor thereof. In some embodiments, the antibody is capable of blocking or impairing the interaction between the immune checkpoint molecule and a ligand or receptor thereof.

In some embodiments, a lesion is treated and/or disrupted to modulate genetically engineered cells in vivo by administering an immunomodulatory agent to the subject. In some embodiments, the immunomodulatory agent blocks, inhibits, or counteracts a component of the immune checkpoint pathway. The immune system has multiple inhibitory pathways that are involved in maintaining self-tolerance and for modulating immune responses. Tumors can use certain immune-checkpoint pathways as a major mechanism of immune resistance, particularly against T cells that are specific for tumor antigens, e.g., engineered cells such as CAR-expressing cells (Pardoll (2012) Nature Reviews Cancer 12:252-264). Because many such immune checkpoints are initiated by ligand-receptor interactions, they can be readily blocked by antibodies against the ligands and/or their receptors. In contrast to the majority of anti-cancer agents, checkpoint inhibitors do not necessarily target tumor cells directly, but rather target lymphocyte receptors or their ligands in order to enhance the endogenous antitumor activity of the immune system.

In particular embodiments, a lesion is treated and/or disrupted by administering an immune checkpoint inhibitor to the subject. In some embodiments, an immune checkpoint inhibitor is a molecule that totally or partially reduces, inhibits, interferes with, or modulates one or more checkpoint proteins. In some embodiments, a checkpoint protein is any protein that regulates T-cell activation or function and/or is responsible for a co-stimulatory or an inhibitory interaction of associated with a T-cell response. Immune checkpoint proteins regulate and maintain self-tolerance and the duration and amplitude of physiological immune responses. Immune checkpoint inhibitors include any agent that blocks, inhibits, or reduces the activity or function of the inhibitory pathways of the immune system. Such inhibitors may include small molecule inhibitors or may include antibodies, or antigen binding fragments thereof, that bind to and block or inhibit immune checkpoint receptors, ligands and/or receptor-ligand interaction. In some embodiments, modulation, enhancement and/or stimulation of particular receptors can overcome immune checkpoint pathway components. Illustrative immune checkpoint molecules that may be targeted for blocking, inhibition, modulation, enhancement and/or stimulation include, but are not limited to, PD-1 (CD279), PD-L1 (CD274, B7-H1), PDL2 (CD273, B7-DC), CTLA-4, LAG-3 (CD223), TIM-3, 4-1BB (CD137), 4-1BBL (CD137L), GITR (TNFRSF18, AITR), CD40, OX40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, γδ, and memory CD8+ (αβ) T cells), CD160 (also referred to as BY55), CGEN-15049, CEACAM (e.g., CEACAM-1, CEACAM-3 and/or CEACAM-5), TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and a transforming growth factor receptor (TGFR; e.g., TGFR beta). Immune checkpoint inhibitors include antibodies, or antigen binding fragments thereof, or other binding proteins that bind to and block or inhibit and/or enhance or stimulate the activity of one or more of any of the said molecules.

Exemplary immune checkpoint inhibitors include Tremelimumab (CTLA-4 blocking antibody, also known as ticilimumab, CP-675,206), anti-OX40, PD-L1 monoclonal antibody (Anti-B7-H1; MEDI4736, also called durvalumab), MK-3475 (PD-1 blocker), nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody) and ipilimumab (anti-CTLA-4 antibody, also known as Yervoy®, MDX-010 and MDX-101). Exemplary of immunomodulatory antibodies include, but are not limited to, Daclizumab (Zenapax), Bevacizumab (AVASTIN®), Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXR0916, AMP-224, MSB0010718C (Avelumab), MEDI4736 (durvalumab), PDR001, rHIgM12B7, Ulocuplumab, BKT140, Vα rlilumab (CDX-1127), ARGX-110, MGA271, lirilumab (BMS-986015, IPH2101), IPH2201, ARGX-115, Emactuzumab, CC-90002 and MNRP1685A or an antibody-binding fragment thereof. Other exemplary immunomodulators include, e.g., afutuzumab (available from ROCHE®); pegfilgrastim (NEULASTA®); lenalidomide (CC-5013, REVELIMID®); thalidomide (THALOMID®), actimid (CC4047); and IRX-2 (mixture of human cytokines including interleukin 1, interleukin 2, and interferon .gamma., CAS 951209-71-5, available from IRX Therapeutics).

In some embodiments, the lesion is treated and/or disrupted by administering an immunomodulatory agent to a subject that binds to and/or inhibits Programmed cell death 1 (PD-1). PD-1 is an immune checkpoint protein that is expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll (2012) Nature Reviews Cancer 12:252-264). The major role of PD-1 is to limit the activity of T cells in peripheral tissues during inflammation in response to infection, as well as to limit autoimmunity. PD-1 expression is induced in activated T cells and binding of PD-1 to one of its endogenous ligands acts to inhibit T-cell activation by inhibiting stimulatory kinases. PD-1 also acts to inhibit the TCR “stop signal”. PD-1 is highly expressed on Treg cells and may increase their proliferation in the presence of ligand (Pardo11 (2012) Nature Reviews Cancer 12:252-264). Anti-PD 1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). In some embodiments, the lesion is treated and/or disrupted by administering an anti-PD-1 antibody, or an antigen binding fragment thereof, to the subject. Exemplary anti-PD-1 antibodies include nivolumab (Opdivo by BMS), pembrolizumab (Keytruda by Merck), pidilizumab (CT-011 by Cure Tech), lambrolizumab (MK-3475 by Merck), and AMP-224 (Merck), nivolumab (also referred to as Opdivo, BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are described in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are described in WO2009/101611. Pembrolizumab (formerly known as lambrolizumab, and also referred to as Keytruda, MK03475; Merck) is a humanized IgG4 monoclonal antibody that binds to PD-1. Pembrolizumab and other humanized anti-PD-1 antibodies are described in U.S. Pat. No. 8,354,509 and WO2009/114335. Other anti-PD-1 antibodies include AMP 514 (Amplimmune), among others, e.g., anti-PD-1 antibodies described in U.S. Pat. No. 8,609,089, US 2010028330, US 20120114649 and/or US 20150210769. AMP-224 (B7-DCIg; Amplimmune; e.g., described in WO2010/027827 and WO2011/066342), is a PD-L2 Fc fusion soluble receptor that blocks the interaction between PD-1 and B7-H1.

In certain embodiments, the lesion is treated and/or disrupted by administering an immunomodulatory agent to the subject that binds to and or inhibits PD-L1 (also known as CD274 and B7-H1) and/or PD-L2 (also known as CD273 and B7-DC). PD-L1 and PD-L2 are ligands for PD-1, found on activated T cells, B cells, myeloid cells, macrophages, and some types of tumor cells. Anti-tumor therapies have focused on anti-PD-L1 antibodies. The complex of PD-1 and PD-L1 inhibits proliferation of CD8⁺ T cells and reduces the immune response (Topalian et al., 2012, N Engl J Med 366:2443-54; Brahmer et al., 2012, N Eng J Med 366:2455-65). Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., 2012, N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). In certain embodiments, the lesion is treated and/or disrupted by administering an anti-PD-L1 antibody, or an antigen binding fragment thereof, to the subject. Exemplary anti-PD-L1 antibodies include MDX-1105 (Medarex), MEDI4736 (durvalumab, Medimmune), MPDL3280A (Genentech), BMS-935559 (Bristol-Myers Squibb) and MSB0010718C.

In some embodiments, the immunomodulatory agent is an anti-PD-L1 antibody. Exemplary of an anti-PD-L1 antibody is MEDI4736 (durvalumab, Medimmune), which is a human monoclonal antibody that binds to PD-L1, and inhibits interaction of the ligand with PD-1 (see U.S. Pat. No. 8,779,108). In some embodiments, the immunomodulatory agent is MDPL3280A (Genentech/Roche), which is a human Fc optimized IgG1 monoclonal antibody that binds to PD-L1. MDPL3280A and other human monoclonal antibodies to PD-L1 are described in U.S. Pat. No. 7,943,743 and U.S Publication No. 20120039906. Other anti-PD-L1 binding agents include YW243.55.570 (see WO2010/077634), MDX-1105 (also referred to as BMS-936559, and, e.g., anti-PD-L1 binding agents described in WO2007/005874), LY3300054 (see US2017/0058033), atezolizumab (see U.S. Pat. No. 8,217,149), and avelumab (U.S. Pat. No. 9.624,298).

In certain embodiments, the lesion is treated and/or disrupted by administering an inhibitor of cytotoxic T-lymphocyte-associated antigen (CTLA-4), also known as CD152. CTLA-4 is a co-inhibitory molecule that functions to regulate T-cell activation. CTLA-4 is a member of the immunoglobulin superfamily that is expressed exclusively on T-cells. CTLA-4 acts to inhibit T-cell activation and is reported to inhibit helper T-cell activity and enhance regulatory T-cell immunosuppressive activity. Although the precise mechanism of action of CTLA-4 remains under investigation, it has been suggested that it inhibits T cell activation by outcompeting CD28 in binding to CD80 and CD86, as well as actively delivering inhibitor signals to the T cell (Pardo11 (2012) Nature Reviews Cancer 12:252-264). Anti-CTLA-4 antibodies have been used in clinical trials for the treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). A significant feature of anti-CTLA-4 is the kinetics of anti-tumor effect, with a lag period of up to 6 months after initial treatment required for physiologic response. In some cases, tumors may actually increase in size after treatment initiation, before a reduction is seen (Pardo11 (2012) Nature Reviews Cancer 12:252-264). In certain embodiments, the lesion is treated and/or disrupted by administering an anti-CTLA-4 antibody, or an antigen binding fragment thereof, to the subject. Exemplary anti-CTLA-4 antibodies include ipilimumab (Bristol-Myers Squibb) and tremelimumab (Pfizer). Ipilimumab has recently received FDA approval for treatment of metastatic melanoma (Wada et al., 2013, J Transl Med 11:89).

In certain embodiments, the lesion is treated and/or disrupted by administering an immunomodulatory agent that binds to and/or inhibits lymphocyte activation gene-3 (LAG-3), also known as CD223. LAG-3 is an immune checkpoint protein that has been associated with the inhibition of lymphocyte activity and in some cases the induction of lymphocyte anergy. LAG-3 is expressed on various cells in the immune system including B cells, NK cells, and dendritic cells. LAG-3 is a natural ligand for the MHC class II receptor, which is substantially expressed on melanoma-infiltrating T cells including those endowed with potent immune-suppressive activity. In certain embodiments, the lesion is treated and/or disrupted by administering an anti-LAG-3 antibody, or an antigen binding fragment thereof, to the subject. Exemplary anti-LAG-3 antibodies include BMS-986016 (Bristol-Myers Squib), which is a monoclonal antibody that targets LAG-3. IMP701 (Immutep) is an antagonist LAG-3 antibody and IMP731 (Immutep and GlaxoSmithKline) is a depleting LAG-3 antibody. Other LAG-3 inhibitors include IMP321 (Immutep), which is a recombinant fusion protein of a soluble portion of LAG-3 and Ig that binds to MHC class II molecules and activates antigen presenting cells (APC). Other antibodies are described, e.g., in WO2010/019570 and US 2015/0259420.

In some embodiments, a lesion is treated and/or disrupted by administering an immunomodulatory agent that binds to and/or inhibits T-cell immunoglobulin domain and mucin domain-3 (TIM-3). TIM-3 initially identified on activated Th1 cells, has been shown to be a negative regulator of the immune response. Blockade of TIM-3 promotes T-cell mediated anti-tumor immunity and has anti-tumor activity in a range of mouse tumor models. Combinations of TIM-3 blockade with other immunotherapeutic agents such as TSR-042, anti-CD137 antibodies and others, can be additive or synergistic in increasing anti-tumor effects. TIM-3 expression has been associated with a number of different tumor types including melanoma, NSCLC and renal cancer, and additionally, expression of intratumoral TIM-3 has been shown to correlate with poor prognosis across a range of tumor types including NSCLC, cervical, and gastric cancers. Blockade of TIM-3 is also of interest in promoting increased immunity to a number of chronic viral diseases. TIM-3 has also been shown to interact with a number of ligands including galectin-9, phosphatidylserine and HMGB1, although which of these, if any, are relevant in regulation of anti-tumor responses is not clear at present. In some embodiments, antibodies, antibody fragments, small molecules, or peptide inhibitors that target TIM-3 can bind to the IgV domain of TIM-3 to inhibit interaction with its ligands. In some embodiments, a lesion is treated and/or disrupted by administering an antibody, or an antigen binding fragment thereof, or a peptide that binds to and/or inhibits TIM-3. Exemplary antibodies and peptides that inhibit TIM-3 are described in US 2015/0218274, WO2013/006490 and US 2010/0247521. Other anti-TIM-3 antibodies include humanized versions of RMT3-23 (Ngiow et al., 2011, Cancer Res, 71:3540-3551), and clone 8B.2C12 (Monney et al., 2002, Nature, 415:536-541). Bi-specific antibodies that inhibit TIM-3 and PD-1 are described in US 2013/0156774.

In some embodiments, the lesion is treated and/or disrupted by administering a CEACAM inhibitor (e.g., CEACAM-1, CEACAM-3, and/or CEACAM-5 inhibitor) to the subject. In certain embodiments, the inhibitor of CEACAM is an anti-CEACAM antibody or antigen binding fragment or variant thereof. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In some embodiments, the anti-CEACAM antibody binds to CEACAM-5 as described in, e.g., Zheng et al. PLoS One. (2011) 6(6): e21146), or cross-reacts with CEACAM-1 and CEACAM-5 as described in, e.g., WO 2013/054331 and US 2014/0271618.

In certain embodiments, the lesion is treated and/or disrupted by administering an immunomodulatory agent that binds to and/or inhibits 4-1BB, also known as CD137. 4-1BB is transmembrane glycoprotein belonging to the TNFR superfamily. 4-1BB receptors are present on activated T cells and B cells and monocytes. In some embodiments, an anti-4-1BB antibody, or antigen binding fragment thereof, is administered to the subject to treat and/or disruptthe lesion. An exemplary anti-4-1BB antibody is urelumab (BMS-663513), which has potential immunostimulatory and antineoplastic activities.

In some embodiments, the lesion is treated and/or disrupted by administering an immunomodulatory agent that is a structural or functional analog or derivative of thalidomide and/or an inhibitor of E3 ubiquitin ligase. In some embodiments, the immunomodulatory agent binds to cereblon (CRBN). In some embodiments, the immunomodulatory agent binds to the CRBN E3 ubiquitin-ligase complex. In some embodiments, the immunomodulatory agent binds to CRBN and the CRBN E3 ubiquitin-ligase complex. In some embodiments, the immunomodulatory agent up-regulates the protein or gene expression of CRBN. In some aspects, CRBN is the substrate adaptor for the CRL4^{CRBN} E3 ubiquitin ligase, and modulates the specificity of the enzyme. In some embodiments, binding to CRB or the CRBN E3 ubiquitin ligase complex inhibits E3 ubiquitin ligase activity. In some embodiments, the immunomodulatory agent induces the ubiqutination of KZF1 (Ikaros) and IKZF3 (Aiolos) and/or induces degradation of IKZF1 (Ikaros) and IKZF3 (Aiolos). In some embodiments, the immunomodulatory agent induces the ubiquitination of casein kinase 1A1 (CK1α) by the CRL4^{CRBN} E3 ubiquitin ligase. In some embodiments, the ubiquitination of CK1α results in CK1α degradation.

In some embodiments, the immunomodulatory agent is an inhibitor of the Ikaros (IKZF1) transcription factor. In some embodiments, the immunomodulatory agent enhances ubiquitination of Ikaros. In some embodiments, the immunomodulatory agent enhances the degradation of Ikaros. In some embodiments, the immunomodulatory agent down-regulates the protein or gene expression of Ikaros. In some embodiments, administration of the immunomodulatory agent causes a decrease in Ikaros protein levels.

In some embodiments, the immunomodulatory agent is an inhibitor of the Aiolos (IKZF3) transcription factor. In some embodiments, the immunomodulatory agent enhances ubiquitination of Aiolos. In some embodiments, the immunomodulatory agent enhances the degradation of Aiolos. In some embodiments, the immunomodulatory agent down-regulates the protein or gene expression of Aiolos. In some embodiments, administration of the immunomodulatory agent causes a decrease in Aiolos protein levels.

In some embodiments, the immunomodulatory agent is an inhibitor of both the Ikaros (IKZF1) and Aiolos (IKZF3) transcription factors. In some embodiments, the immunomodulatory agent enhances ubiquitination of both Ikaros and Aiolos. In some embodiments, the immunomodulatory agent enhances the degradation of both Ikaros and Aiolos. In some embodiments, the immunomodulatory agent enhances ubiquitination and degradation of both Ikaros and Aiolos. In some embodiments, administration of the immunomodulatory agent causes both Aiolos protein levels.and Ikaros protein levels to decrease.

In some embodiments, the immunomodulatory agent is a selective cytokine inhibitory drug (SelCID). In some embodiments, the immunomodulatory agent inhibits the activity of phosphodiesterase-4 (PDE4). In some embodiments, the immunomodulatory agent suppresses the enzymatic activity of the CDC25 phosphatases. In some embodiments, the immunomodulatory agent alters the intracellular trafficking of CDC25 phosphatases.

In some embodiments, the immunomodulatory agent is thalidomide (2-(2,6-dioxopiperidin-3-yl)-1H-isoindole-1,3(2H)-dione) or an analog or derivative of thalidomide. In certain embodiments, a thalidomide derivative includes structural variants of thalidomide that have a similar biological activity. Exemplary thalidomide derivatives include, but are not limited to lenalidomide (REVLIMMUNOMODULATORY COMPOUND™; Celgene Corporation), pomalidomide (also known as ACTIMMUNOMODULATORY COMPOUND™ or POMALYST™; Celgene Corporation), CC-1088, CDC-501, and CDC-801, and the compounds disclosed in U.S. Pat. Nos. 5,712,291; 7,320,991; and 8,716,315; U.S. Appl. No. 2016/0313300; and PCT Pub. Nos. WO 2002/068414 and WO 2008/154252.

In some embodiments, the immunomodulatory agent is 1-oxo- and 1,3 dioxo-2-(2,6-dioxopiperldin-3-yl) isoindolines substituted with amino in the benzo ring as described in U.S. Pat. No. 5,635,517 which is incorporated herein by reference.

In some embodiments, the immunomodulatory agent is a compound of the following formula:

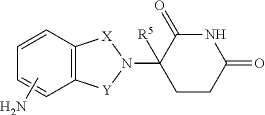

wherein one of X and Y is —C(O)— and the other of X and Y is —C(O)— or —CH₂—, and R⁵ is hydrogen or lower alkyl, or a pharmaceutically acceptable salt thereof. In some embodiments, X is —C(O)— and Y is —CH₂—. In some embodiments, both X and Y are —C(O)—. In some embodiments, R⁵ is hydrogen. In other embodiments, R⁵ is methyl.

In some embodiments, the immunomodulatory compound is a compound that belongs to a class of substituted 2-(2, 6-dioxopiperidin-3-yl)phthalimmunomodulatory compounds and substituted 2-(2,6-dioxopiperldin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference.

In some embodiments, the immunomodulatory agent is a compound of the following formula:

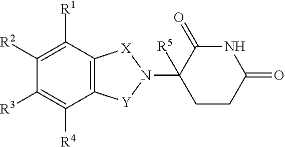

wherein
one of X and Y is —C(O)— and the other of X and Y is —C(O)— or —CH₂—;
(1) each of R¹, R², R³, and R⁴ are independently halo, alkyl of 1 to 4 carbon atoms, or alkoxy or 1 to 4 carbon atoms, or
(2) one of R¹, R³, R⁴, and R⁵ is —NHR^{a} and the remaining of R¹, R², R³, and R⁴ is are hydrogen, wherein R^{a} is hydrogen or alkyl of 1 to 8 carbon atoms;
R⁵ is hydrogen or alkyl of 1 to 8 carbon atoms, benzyl, or halo;
provided that R⁵ is other than hydrogen if X and Y are —C(O)— and (i) each of R¹, R², R³, and R⁴ is fluoro; or (ii) one of R¹, R², R³, and R⁴ is amino;
or a pharmaceutically acceptable salt thereof.

In some embodiments, the immunomodulatory agent is a compound that belongs to a class of isoindole-immunomodulatory compounds disclosed in U.S. Pat. No. 7,091,353, U.S. Patent Publication No. 2003/0045552, and International Application No. PCT/USOI/50401 (International Publication No. WO02/059106), each of which are incorporated herein by reference. For example, in some embodiments, the immunomodulatory agent is [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N—(2-(2,6-dioxo-piperidin-3-yl)-1,3-dioxo-2,3-dihydro-1H-isoindol-4-ylmethyl)-acetamide; N—{(2-(2,6-dioxo(3-piperidyl)-1,3-dioxoisoindolin-4-yl)methyl)cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}(-2-furylcarboxamide; {N-(2-(2,6-dioxo(3 -piperidyl))-1,3-dioxoisoindolin-4-yl)carbamoyl}methyl acetate; N—(2-(2,6-dioxo(3-piperidyl))-1,3-dioxoisoindolin-4-yl)pentanamide; N-—(2-(2,6-dioxo(3-piperidyl))-1,3-dioxoisoindolin-4-yl)-2-thienylcarboxamide; N—{[2-(2,6-dioxo(3-piperidyl))-1,3-dioxoisoindolin-4-yl]methyl}(butylamino)carboxamide; N—{[2-(2,6-dioxo(3-piperidyl))-1,3-dioxoisoindolin-4-yl]methyl}(octylamino)carboxamide; or N—{[2-(2,6-dioxo(3-piperidyl))-1,3-dioxoisoindolin-4-yl]methyl}(benzylamino)carboxamide.

In some embodiments, the immunomodulatory agent is a compound that belongs to a class of isoindole-immunomodulatory compounds disclosed in U.S. Patent Application Publication Nos. 2002/0045643, International Publication No. WO 98/54170, and U.S. Pat. No. 6,395,754, each of which is incorporated herein by reference. In some embodiments, the immunomodulatory agent is a tetra substituted 2-(2,6-dioxopiperdin-3-yl)-1-oxoisoindolines described in U.S. Pat. No. 5,798,368, which is incorporated herein by reference. In some embodiments, the immunomodulatory agent is 1-oxo and 1,3-dioxo-2-(2,6-dioxopiperidin-3-yl) isoindolines disclosed in U.S. Pat. No. 6,403,613, which is incorporated herein by reference. In some embodiments the immunomodulatory agent is a 1-oxo or 1,3-dioxoisoindoline substituted in the 4- or 5-position of the indoline ring as described in U.S. Pat. No. 6,380,239 and U.S. Pat. No. 7,244,759, both of which are incorporated herein by reference.

In some embodiments, the immunomodulatory agent is 2-(4-amino-1-oxo-1,3-dihydro-isoindo2-yl)-4-carbamoyl-butyric acid or 4-(4-amino-1-oxo-1,3-dihydro-isoindol-2-yl)-4-carbamoyl-butyric acid. In some embodiments, the immunomodulatory compound is 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, or 2-{4-[(furan-2-yl-methyl)-amino]-1,3-dioxo-1,3-dihydro-isoindol-2-yl}-pentanedioic acid.

In some embodiments, the immunomodulatory agent is a isoindoline-1-one or isoindoline-1,3-dione substituted in the 2-position with 2,6-dioxo-3-hydroxypiperidin-5-yl as described in U.S. Pat. No. 6,458,810, which is incorporated herein by reference. In some embodiments, the immunomodulatory compound is 3-(5-amino-2-methyl-4-oxo-4H-quinazolin-3-yl)-piperidine-2,6-dione, or an enantiomer or a mixture of enantiomers thereof; or a pharmaceutically acceptable salt, solvate, hydrate, co-crystal, clathrate, or polymorph thereof. In some embodiments, the immunomodulatory compound is 3-[4-(4-morpholin-4-ylmethyl-benzyloxy)-1-oxo-1,3-dihydro-isoindol-2-yl]-piperidine-2,6-dione.

In some embodiments, the immunomodulatory agent is as described in Oshima, K. et al., Nihon Rinsho., 72(6):1130-5 (2014); Millrine, D. et al., Trends Mol Med., 23(4):348-364 (2017); and Collins, et al., Biochem J., 474(7):1127-1147 (2017).

In some embodiments, the immunomodulatory agent is lenalidomide, pomalidomide, avadomide, a stereoisomer of lenalidomide, pomalidomide, avadomide or a pharmaceutically acceptable salt, solvate, hydrate, co-crystal, clathrate, or polymorph thereof. In some embodiments, the immunomodulatory compound is lenalidomide, a stereoisomer of lenalidomide or a pharmaceutically acceptable salt, solvate, hydrate, co-crystal, clathrate, or polymorph thereof. In some embodiments, the immunomodulatory compound is lenalidomide, or ((RS)-3-(4-Amino-1-oxo-1,3-dihydro-2H-isoindol-2-yl)piperidine-2,6-dione).

In certain embodiments, the lesion is treated and/or disrupted by administering the thalidomide derivative lenalidomide, ((RS)-3-(4-Amino-1-oxo-1,3-dihydro-2H-isoindol-2-yl)piperidine-2,6-dione) to the subject. Lenalidomide is FDA approved for the treatment of multiple myeloma, myelodysplastic syndrome associated with deletion 5q, and most recently in relapsed/refractory mantle-cell lymphoma (MCL). Lenalidomide generally is a synthetic derivative of thalidomide, and is currently understood to have multiple immunomodulatory effects, including enforcement of immune synapse formation between T cell and antigen presenting cells (APCs). For example, in some cases, lenalidomide modulates T cell responses and results in increased interleukin (IL)-2 production in CD4⁺ and CD8⁺ T cells, induces the shift of T helper (Th) responses from Th2 to Th1, inhibits expansion of regulatory subset of T cells (Tregs), and improves functioning of immunological synapses in follicular lymphoma and chronic lymphocytic leukemia (CLL) (Otahal et al., Oncoimmunology (2016) 5(4):e1115940). Lenalidomide also has direct tumoricidal activity in patients with multiple myeloma (MM) and directly and indirectly modulates survival of CLL tumor cells by affecting supportive cells, such as nurse-like cells found in the microenvironment of lymphoid tissues. Lenalidomide also can enhance T-cell proliferation and interferon-y production in response to activation of T cells via CD3 ligation or dendritic cell-mediated activation. In addition, lenalidomide is thought to decrease proliferation of pro-inflammatory cytokines including TNF-α, IL-1, IL-6, and IL-12 and enhance antibody-dependent cellular cytotoxicity (ADCC) via increased NK cell activation. Lenalidomide can also induce malignant B cells to express higher levels of immunostimulatory molecules such as CD80, CD86, HLA-DR, CD95, and CD40 (Fecteau et al., Blood (2014) 124(10):1637-1644). Cereblon, an E3 ubiquitin ligase, was identified as the primary target for thalidomide-induced teratogenesis (Ito et al., T., (2010) Science 327: 1345-1350). Lenalidomide also targets cereblon and it has been shown that this leads to the reduction of c-Myc and IRF4 expression while also increasing expression of p21 that leads to G1 cell-cycle arrest (Lopez-Girona et al., (2012) Leukemia 26: 2326-2335).

In some embodiments, the lesion is treated and/or disrupted by administering an agent that modulates adenosine levels and/or modulates the activity or amount of an adenosine pathway component. Adenosine can function as an immunomodulatory agent in the body. For example, adenosine and some adenosine analogs that non-selectively activate adenosine receptor subtypes decrease neutrophil production of inflammatory oxidative products (Cronstein et al., Ann. N.Y. Acad. Sci. 451:291, 1985; Roberts et al., Biochem. J., 227:669, 1985; Schrier et al., J. Immunol. 137:3284, 1986; Cronstein et al., Clinical Immunol. Immunopath. 42:76, 1987). In some cases, concentration of extracellular adenosine or adenosine analogs can increase in specific environments, e.g., tumor microenvironment (TME). In some cases, adenosine or adenosine analog signaling depends on hypoxia or factors involved in hypoxia or its regulation, e.g., hypoxia inducible factor (HIF). In some embodiments, increase in adenosine signaling can increase in intracellular cAMP and cAMP-dependent protein kinase that results in inhibition of proinflammatory cytokine production, and can lead to the synthesis of immunosuppressive molecules and development of Tregs (Sitkovsky et al., Cancer Immunol Res (2014) 2(7):598-605). In some embodiments, the additional agent can reduce or reverse immunosuppressive effects of adenosine, adenosine analogs and/or adenosine signaling. In some embodiments, the additional agent can reduce or reverse hypoxia-driven A2-adenosinergic T cell immunosuppression. In some embodiments, the additional agent is selected from among antagonists of adenosine receptors, extracellular adenosine-degrading agents, inhibitors of adenosine generation by CD39/CD73 ectoenzymes, and inhibitors of hypoxia-HIF-la signaling. In some embodiments, the additional agent is an adenosine receptor antagonist or agonist.

In particular embodiments, an agent that inhibits or reduces extracellular adenosine is administered to the subject to treat and/or disrupt the lesion. In some embodiments, an agent that inhibits the activity and/or an amount of an adenosine receptor is administered to the subject to treat and/or disrupt the lesion. Particular embodiments contemplate that inhibition or reduction of extracellular adenosine or the adenosine receptor by virtue of an inhibitor of extracellular adenosine (such as an agent that prevents the formation of, degrades, renders inactive, and/or decreases extracellular adenosine), and/or an adenosine receptor inhibitor (such as an adenosine receptor antagonist) can enhance immune response, such as a macrophage, neutrophil, granulocyte, dendritic cell, T- and/or B cell-mediated response. In addition, inhibitors of the Gs protein mediated cAMP dependent intracellular pathway and inhibitors of the adenosine receptor-triggered Gi protein mediated intracellular pathways, can also increase acute and chronic inflammation.

In some embodiments, an adenosine receptor antagonist is administered to the subject to treat and/or disrupt the lesion. In particular embodiments, an adenosine receptor antagonist is administered to the subject to treat and/or disrupt the lesion. In some embodiments, the adenosine receptor antagonist is an A2a, A2b, and/or an A3 antagonist. A2a, A2b, and A3 receptors can suppress or reduce the immune response, therefore antagonizing immunosuppressive adenosine receptors can augment, boost or enhance immune response. In some embodiments, an agent that inhibits the production of extracellular adenosine and/or inhibits adenosine-triggered signaling through adenosine receptors is administered to the subject to treat and/or disrupt the lesion. In some embodiments, an immune response to the lesion, tissue inflammation of the lesion, and targeted tissue destruction of the lesion can be enhanced by inhibiting or reducing the adenosine-producing local tissue hypoxia; by degrading (or rendering inactive) accumulated extracellular adenosine; by preventing or decreasing expression of adenosine receptors on immune cells; and/or by inhibiting signaling by adenosine ligands through adenosine receptors.

In particular embodiments, an adenosine receptor antagonist is administered to subject to treat and/or disrupt a lesion. In some embodiments, the antagonist is a small molecule adenosine receptor antagonist, such as an A2a, A2b, or A3 receptor antagonist. In some embodiments, the antagonist is a peptide, or a pepidomimetic, that binds the A2a, A3b, and/or A3 adenosine receptor but does not trigger a Gᵢ protein dependent intracellular signaling pathway. Examples of such antagonists are described in U.S. Pat. Nos. 5,565,566; 5,545,627, 5,981,524; 5,861,405; 6,066,642; 6,326,390; 5,670,501; 6,117,998; 6,232,297; 5,786,360; 5,424,297; 6,313,131, 5,504,090; and 6,322,771.

In some embodiments, an A2 receptor (A2R) antagonist is administered to the subject to treat and/or disrupt the lesion. Exemplary A2R antagonists include, but are not limited to, KW6002 (istradefyline), SCH58261, caffeine, paraxanthine, 3,7-dimethyl-1-propargylxanthine (DMPX), 8-(m-chlorostyryl) caffeine (CSC), MSX-2, MSX-3, MSX-4, CGS-15943, ZM-241385, SCH-442416, preladenant, vipadenant (BII014), V2006, ST-1535, SYN-115, PSB-1115, ZM241365, FSPTP, and an inhibitory nucleic acid targeting A2R expression, e.g., siRNA or shRNA, or any antibodies or antigen-binding fragment thereof that targets an A2R. In some embodiments, the additional agent is an A2R antagonist described in, e.g., Ohta et al., Proc Natl Acad Sci U S A (2006) 103:13132-13137; Jin et al., Cancer Res. (2010) 70(6):2245-2255; Leone et al., Computational and Structural Biotechnology Journal (2015) 13:265-272; Beavis et al., Proc Natl Acad Sci U S A (2013) 110:14711-14716; and Pinna, A., Expert Opin Investig Drugs (2009) 18:1619-1631; Sitkovsky et al., Cancer Immunol Res (2014) 2(7):598-605; U.S. Pat. No. 8,080,554; U.S. Pat. No. 8,716,301; US 20140056922; WO2008/147482; U.S. Pat. No. 8,883,500; US 20140377240; WO02/055083; US 7,141,575; US 7,405,219; U.S. Pat. No. 8,883,500; U.S. Pat. No. 8,450,329 and U.S. Pat. No. 8,987,279).

In particular embodiments, an adenosine receptor antagonist that is an antisense molecule, an inhibitory nucleic acid molecule (e.g., small inhibitory RNA (siRNA)) or a catalytic nucleic acid molecule (e.g. a ribozyme) that specifically binds mRNA encoding an adenosine receptor is administered to the subject to treat and/or disrupt a lesion. In some embodiments, the antisense molecule, inhibitory nucleic acid molecule or catalytic nucleic acid molecule binds nucleic acids encoding A2a, A2b, or A3. In some embodiments, an antisense molecule, inhibitory nucleic acid molecule or catalytic nucleic acid targets biochemical pathways downstream of the adenosine receptor. For example, the antisense molecule or catalytic nucleic acid can inhibit an enzyme involved in the Gₛ protein- or Gᵢ protein-dependent intracellular pathway. In some embodiments, the additional agent includes dominant negative mutant form of an adenosine receptor, such as A2a, A2b, or A3.

In some embodiments, the lesion is treated and/or disrupted by administering an agent that inhibits extracellular adenosine to the subject. Agents that inhibit extracellular adenosine include agents that render extracellular adenosine non-functional (e.g. render extracellular adenosine unable to bind to and/or activate an adenosine receptor), such as a substance that modifies the structure of extracellular adenosine. In some embodiments, the additional agent is an extracellular adenosine-generating or adenosine-degrading enzyme, a modified form thereof or a modulator thereof. For example, in some embodiments, the additional agent is an enzyme (e.g. adenosine deaminase) or another catalytic molecule that selectively binds and destroys the adenosine, thereby abolishing or decreasing the ability of endogenously formed adenosine to signal through adenosine receptors and terminate inflammation.

In certain embodiments, the lesion is treated and/or disrupted by administering an adenosine deaminase (ADA) or a modified form thereof, e.g., recombinant ADA and/or polyethylene glycol-modified ADA (ADA-PEG), to the subject. Adenosine deaminase can inhibit local tissue accumulation of extracellular adenosine. ADA-PEG has been used in treatment of patients with ADA SCID (Hershfield (1995) Hum Mutat. 5:107). In some embodiments, an agent that inhibits extracellular adenosine is administered to the subject that includes agents that prevent or decrease formation of extracellular adenosine, and/or prevent or decrease the accumulation of extracellular adenosine, thereby abolishing, or substantially decreasing, the immunosuppressive effects of adenosine. In some embodiments, an agent is administered to the subject that specifically inhibits enzymes and proteins that are involved in regulation of synthesis and/or secretion of pro-inflammatory molecules, including modulators of nuclear transcription factors. Suppression of adenosine receptor expression or expression of the Gₛ protein- or Gᵢ protein-dependent intracellular pathway, or the cAMP dependent intracellular pathway, can result in an increase/enhancement of immune response.

In some embodiments, an agent that targets ectoenzymes that generate or produce extracellular adenosine is administered to the subject to treat and/or disrupt the lesion. In some embodiments, the agent targets CD39 and CD73 ectoenzymes, which function in tandem to generate extracellular adenosine. CD39 (also called ectonucleoside triphosphate diphosphohydrolase) converts extracellular ATP (or ADP) to 5′AMP. Subsequently, CD73 (also called 5′nucleotidase) converts 5′AMP to adenosine. The activity of CD39 is reversible by the actions of NDP kinase and adenylate kinase, whereas the activity of CD73 is irreversible. CD39 and CD73 are expressed on tumor stromal cells, including endothelial cells and Tregs, and also on many cancer cells. For example, the expression of CD39 and CD73 on endothelial cells is increased under the hypoxic conditions of the tumor microenvironment. Tumor hypoxia can result from inadequate blood supply and disorganized tumor vasculature, impairing delivery of oxygen (Carroll and Ashcroft (2005), Expert. Rev. Mol. Med. 7(6):1-16). Hypoxia also inhibits adenylate kinase (AK), which converts adenosine to AMP, leading to very high extracellular adenosine concentration. Thus, adenosine is released at high concentrations in response to hypoxia, which is a condition that frequently occurs the tumor microenvironment (TME), in or around solid tumors. In some embodiments, the additional agent is one or more of anti-CD39 antibody or antigen binding fragment thereof, anti-CD73 antibody or antigen binding fragment thereof, e.g., MEDI9447 or TY/23, α-β-methylene-adenosine diphosphate (ADP), ARL 67156, POM-3, IPH52 (see, e.g., Allard et al. Clin Cancer Res (2013) 19(20):5626-5635; Hausler et al., Am J Transl Res (2014) 6(2):129-139; Zhang, B., Cancer Res. (2010) 70(16):6407-6411).

In some embodiments, a chemotherapeutic agent (sometimes referred to as a cytotoxic agent) is administered to the subject to treat and/or disrupt a lesion. In certain embodiments, the lesion is tumor. In particular embodiments, the lesion is cancerous. In particular embodiments, the chemotherapeutic agent is any agent known to those of skill in the art to be effective for the treatment, prevention or amelioration of hyperproliferative disorders such as cancer. Chemotherapeutic agents include, but are not limited to, small molecules, synthetic drugs, peptides, polypeptides, proteins, nucleic acids (e.g., DNA and RNA polynucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. In particular embodiments, chemotherapeutic drugs include alkylating agents, anthracyclines, cytoskeletal disruptors (taxanes), epothilones, histone deacetylase inhibitors, topoisomerase inhibitors, topoisomerase II inhibitors, kinase inhibitors, nucleotide analogs and precursor analogs, peptide antibiotics, platinum-based agents, and vinca alkaloids and derivatives.

In certain embodiments, a lesion is treated and/or disrupted by administering a chemotherapeutic agent to modulate genetically engineered cells in vivo. Chemotherapeutic agents may include, but are not limited to, abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

C. Re-Expansion of Genetically Engineered Cells

In some embodiments, the provided methods promote re-expansion of the engineered cells in the subject, which, in some cases, can far exceed the initial peak level of expansion prior to the treatment and/or disruption. In some embodiments, the provided methods modulate expansion and/or persistence of genetically engineered T cells at times when the peak levels of the engineered cells has declined or is not detectable. In some embodiments, a genetically engineered cell induced to re-expand exhibit better potency in a subject to which it is administered.

Methods for monitoring or detecting CAR+ T cells are known and exemplary methods are described in Section IV.C. In some embodiments, the degree or extent of expansion of administered cells can be detected or quantified after administration to a subject. For example, in some aspects, quantitative PCR (qPCR) is used to assess the quantity of cells expressing the chimeric receptor (e.g., CAR-expressing cells) in the blood or serum or organ or tissue (e.g., disease site) of the subject. In some aspects, expansion, including numbers of engineered cells, is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the recombinant receptor (e.g. CAR-expressing cells) can be used to distinguish the administered cells from endogenous cells in a subject.

In some embodiments, disrupting the lesion in accord with the provided methods promotes activation, re-expansion and/or increased exposure of the subject to the cells, e.g., T cells administered for T cell based therapy, such as by promoting their expansion and/or persistence over time. In some embodiments, the T cell therapy exhibits increased or prolonged expansion and/or persistence in the subject, or on average in a plurality of subjects so-treated, as compared to a method in which the T cell therapy is administered to the subject(s) in the absence of disrupting the lesion. In some embodiments, the provided methods involving disrupting the lesion in vivo can increase the maximum, total, and/or duration of exposure to the cells, e.g. T cells administered for the T cell based therapy, in the subject, or on average in a plurality of subjects so treated, as compared to administration of the T cells alone in the absence of disrupting the lesion. Such increases can be by at or about or at least about 1.2-fold, 1.5-fold, 2.0-fold, 3.0-fold, 4.0-fold, 5.0-fold, 6.0-fold, 7.0-fold, 8.0-fold, 9.0-fold, 10.0-fold, 20.0-fold, 30.0-fold, 40.0-fold, 50.0-fold or more.

In some embodiments, increased exposure of the subject to the administered cells (e.g., increased number of cells or duration over time) as achieved by the provided methods improve efficacy and therapeutic outcomes of the immunotherapy, e.g. T cell therapy. In some aspects, the methods are advantageous in that a greater and/or longer degree of exposure to the cells expressing the recombinant receptors, e.g., CAR-expressing cells, improves treatment outcomes as compared with other methods. Such outcomes may include patient survival and remission, even in individuals with severe tumor burden. In some aspects, the increased or prolonged expansion and/or persistence of the dose of cells in the subject, or on average in a plurality of subjects so-treated, that is achieved following the treatment and/or disruption of the lesion is associated with a benefit in tumor related outcomes in the subject(s). In some embodiments, the tumor related outcome includes a decrease in tumor burden or a decrease in blast marrow in the subject(s). In some embodiments, the tumor burden is decreased by or by at least at or about 10, 20, 30, 40, 50, 60, 70, 80, 90, or 100 percent after administration of the method. In some embodiments, disease burden, tumor size, tumor volume, tumor mass, and/or tumor load or bulk is reduced following the dose of cells by at least at or about 50%, 60%, 70%, 80%, 90% or more compared a subject, or on average a plurality of subjects so treated, that has been treated with a method that does not involve disrupting the lesion.

In some embodiments, the provided methods effectively treats the subject despite the subject having become resistant to another therapy and/or having relapsed following administration of the engineered cells, such as recombinant receptor-expressing cells, e.g. CAR+ T cells. In some embodiments, criteria assessed for effective treatment includes overall response rate (ORR), complete response (CR), duration of response (DOR) progression-free survival (PFS), and/or overall survival (OS). In some embodiments, the methods and uses provide for or achieve more durable responses in a subject, or on average in a plurality of subjects so-treated, compared to a method that does not involve disrupting the lesion. In particular embodiments of any of the provided methods, the response, for example ORR or CR, is durable for greater than 3 months, greater than 6 months, greater than 12 months, greater than 18 months, greater than 24 months, greater than 30 months, greater than 36 months or more following disrupting of the lesion and/or administering a pharmacologic or therapeutic agent in accord with the provided methods.

### II. CELL THERAPY AND ENGINEERED CELLS

The provided therapeutic methods involve administering cells expressing a recombinant receptor, and compositions thereof, to subjects, e.g., patients. In some embodiments, the cells contain or are engineered to contain an engineered receptor, e.g., an engineered antigen receptor, such as a chimeric antigen receptor (CAR), or a T cell receptor (TCR). The cells include populations of such cells, compositions containing such cells and/or enriched for such cells, such as in which cells of a certain type such as T cells or CD8⁺ or CD4⁺ cells are enriched or selected. Among the compositions are pharmaceutical compositions and formulations for administration, such as for adoptive cell therapy.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

A. Recombinant Receptors

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors, such as chimeric autoantibody receptors (CAARs)

1. Chimeric Antigen Receptors (CARS)

In some embodiments, the recombinant receptor includes a chimeric antigen receptor (CAR). In some embodiments, the CAR is specific for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as a chimeric receptor, contains an intracellular signaling region, which includes a cytoplasmic signaling domain (also interchangeably called an intracellular signaling domain), such as a cytoplasmic (intracellular) region capable of inducing a primary activation signal in a T cell, for example, a cytoplasmic signaling domain of a T cell receptor (TCR) component (e.g. a cytoplasmic signaling domain of a zeta chain of a CD3-zeta (CD3) chain or a functional variant or signaling portion thereof) and/or that comprises an immunoreceptor tyrosine-based activation motif (ITAM).

In some embodiments, the chimeric receptor further contains an extracellular binding domain that specifically binds to an antigen (or a ligand). In some embodiments, the chimeric receptor is a CAR that contains an extracellular antigen-recognition domain that specifically binds to an antigen. In some embodiments, the antigen (or a ligand), is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs, and methods for engineering and introducing such receptors into cells, include those described, for example, in International Patent Application Publication Numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos.: 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416,and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No.: 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Patent No.: 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat Nos. 7,446,190, 8,389,282. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g. CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues.

In some embodiments, the CAR contains an antibody or an antigen-binding fragment (e.g. scFv) that specifically recognizes an antigen, such as an intact antigen, expressed on the surface of a cell.

Antigens targeted by the receptors in some embodiments are or include orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, and MAGE A3, CE7, Wilms Tumor 1 (WT-1), a cyclin, such as cyclin A1(CCNA1), G Protein Coupled Receptor 5D (GPCR5D), and/or biotinylated molecules, and/or molecules expressed by HIV, HCV, HBV or other pathogens.

In certain embodiments, the engineered cell expresses a recombinant receptor and/or a CAR that binds to an antigen. In particular embodiments, the antigen is αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acetylcholine receptor, ganglioside GD2, 0-acetylated GD2 (OGD2), ganglioside GD3, glycoprotein 100 (gp100), Her2/neu (receptor tyrosine kinase erbB2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Ra), IL-13 receptor alpha 2 (IL-13Ra2), kinase insert domain receptor (kdr), kappa light chain, L1 cell adhesion molecule (L1CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), G Protein Coupled Receptor 5D (GPCR5D), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the CAR binds a pathogen-specific or pathogen-expressed antigen. In some embodiments, the CAR is specific for viral antigens (such as HIV, HCV, HBV, etc.), bacterial antigens, and/or parasitic antigens.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g. US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US published application No. US20020150914, US2014/0294841; and Cohen CJ. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W. H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the recombinant receptor such as the CAR, including the antibody portion of the recombinant receptor, e.g., CAR, further includes at least a portion of an immunoglobulin constant region, such as a hinge region, e.g., an IgG4 hinge region, and/or a C_{H}1/C_{L} and/or Fc region. In some embodiments, the recombinant receptor such as the CAR, including the antibody portion thereof, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers, e.g., hinge regions, include those described in International Patent Application Publication Number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to C_{H}2 and C_{H}3 domains, or IgG4 hinge linked to the C_{H}3 domain. Exemplary spacers include, but are not limited to, those described in Hudecek et al. (2013) Clin. Cancer Res., 19:3153, International Patent Application Publication Number WO2014031687, U.S. Pat. No. 8,822,647 or published app. No. US2014/0271635.

In some embodiments, the constant region or portion is of a human IgG, such as IgG4 or IgG1. In some embodiments, the spacer has the sequence ESKYGPPCPPCP (set forth in SEQ ID NO: 1), and is encoded by the sequence set forth in SEQ ID NO: 2. In some embodiments, the spacer has the sequence set forth in SEQ ID NO: 3. In some embodiments, the spacer has the sequence set forth in SEQ ID NO: 4. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 or 5. In some embodiments, the spacer has the sequence set forth in SEQ ID NOS: 26-34. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 26-34.

The antigen recognition domain generally is linked to one or more intracellular signaling components, such as signaling components that mimic activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains or regions. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains or regions are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain or region of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domains or regions of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain or region of an antigen receptor component or costimulatory molecule is used in place of an intact immunostimulatory chain, for example, if it transduces the effector function signal. In some embodiments, the intracellular signaling domain or domains or regions include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CD8, CD22, CD79a, CD79b, and CD66d. In some embodiments, cytoplasmic signaling molecule(s) in the CAR contain(s) a cytoplasmic signaling domain or region, portion thereof, or sequence derived from CD3 zeta.

In some embodiments, the CAR includes a signaling domain or region and/or transmembrane portion of a costimulatory receptor, such as CD28, 4-1BB, OX40, DAP10, and ICOS. In some aspects, the same CAR includes both the activating and costimulatory components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in International Patent Application Publication Number WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 16 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 7 or 16. An exemplary T2A linker sequence comprises the sequence of amino acids set forth in SEQ ID NO: 6 or 17 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 6 or 17.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments the scFv is derived from FMC63. FMC63 is a mouse monoclonal IgG1 antibody raised against Nalm-1 and -16 cells expressing CD19 of human origin (Ling, N. R., et al. (1987). Leucocyte typing III. 302). The FMC63 antibody comprises CDRH1and H2 set forth in SEQ ID NOS: 38 and 39 respectively, and CDRH3 set forth in SEQ ID NOS: 40 or 54 and CDRL1 set forth in SEQ ID NOS: 35 and CDR L2 set forth in SEQ ID NOS: 36 or 55 and CDR L3 set forth in SEQ ID NOS: 37 or 56. The FMC63 antibody comprises the heavy chain variable region (V_{H}) comprising the amino acid sequence of SEQ ID NO: 41 and the light chain variable region (V_{L}) comprising the amino acid sequence of SEQ ID NO: 42. In some embodiments, the svFv comprises a variable light chain containing the CDRL1 set forth in SEQ ID NO: 35, a CDRL2 set forth in SEQ ID NO: 36 or 55, and a CDRL3 set forth in SEQ ID NO: 37 or 56 and/or a variable heavy chain containing a CDRH1 set forth in SEQ ID NO:38, a CDRH2 set forth in SEQ ID NO:39, and a CDRH3 set forth in SEQ ID NO:40 or 54. In some embodiments, the scFv comprises a variable heavy chain region of FMC63 set forth in SEQ ID NO:41 and a variable light chain region of FMC63 set forth in SEQ ID NO: 42. In some embodiments, the variable heavy and variable light chain are connected by a linker. In some embodiments, the linker is set forth in SEQ ID NO:24. In some embodiments, the scFv comprises, in order, a VH, a linker, and a VL. In some embodiments, the scFv comprises, in order, a VL, a linker, and a VH. In some embodiments, the svFc is encoded by a sequence of nucleotides set forth in SEQ ID NO:25 or a sequence that exhibits at least or at least about 85%, 86%, 87%, 88%, 89%, 90%, 91%, 92%, 93%, 94%, 95%, 96%, 97%, 98%, or 99% sequence identity to SEQ ID NO:25. In some embodiments, the scFv comprises the sequence of amino acids set forth in SEQ ID NO:43 or a sequence that exhibits at least or at least about 85%, 86%, 87%, 88%, 89%, 90%, 91%, 92%, 93%, 94%, 95%, 96%, 97%, 98%, or 99% sequence identity to SEQ ID NO:43.

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). The SJ25C1 antibody comprises CDRH1, H2 and H3 set forth in SEQ ID NOS: 47-49, respectively, and CDRL1, L2 and L3 sequences set forth in SEQ ID NOS: 44-46, respectively. The SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51. In some embodiments, the svFv comprises a variable light chain containing the CDRL1 set forth in SEQ ID NO: 44, a CDRL2 set forth in SEQ ID NO: 45, and a CDRL3 set forth in SEQ ID NO:46 and/or a variable heavy chain containing a CDRH1 set forth in SEQ ID NO: 47, a CDRH2 set forth in SEQ ID NO: 48, and a CDRH3 set forth in SEQ ID NO:49. In some embodiments, the scFv comprises a variable heavy chain region of SJ25C1 set forth in SEQ ID NO:50 and a variable light chain region of SJ25C1 set forth in SEQ ID NO: 51. In some embodiments, the variable heavy and variable light chain are connected by a linker. In some embodiments, the linker is set forth in SEQ ID NO:52. In some embodiments, the scFv comprises, in order, a VH, a linker, and a VL. In some embodiments, the scFv comprises, in order, a VL, a linker, and a VH. In some embodiments, the scFv comprises the sequence of amino acids set forth in SEQ ID NO:53 or a sequence that exhibits at least or at least about 85%, 86%, 87%, 88%, 89%, 90%, 91%, 92%, 93%, 94%, 95%, 96%, 97%, 98%, or 99% sequence identity to SEQ ID NO:53.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P01747.1) or variant thereof, such as a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 8; in some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto, or such as a 27-amino acid transmembrane domain of a human CD28.

In some embodiments, the chimeric antigen receptor contains an intracellular domain of a T cell costimulatory molecule. In some aspects, the T cell costimulatory molecule is CD28 or 4-1BB.

In some embodiments, the intracellular signaling domain or region, region or component(s) of the recombinant receptor, e.g. the CAR, contains an intracellular costimulatory signaling domain or region of human CD28 or a functional variant or portion thereof, such as a domain or region with an LL to GG substitution at positions 186-187 of a native CD28 protein. For example, in some embodiments, the intracellular signaling domain or region can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular domain or region comprises an intracellular costimulatory signaling domain or region of 4-1BB ((e.g.,Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12 or such as a 42-amino acid cytoplasmic domain of a human 4-1BB.

In some embodiments, the intracellular signaling domain or region of the recombinant receptor, e.g. the CAR, comprises a human CD3 chain, optionally a zeta stimulatory signaling domain or region or functional variant thereof, such as an 112 AA cytoplasmic domain or region of isoform 3 of human CD3 (Accession No.: P20963.2) or a CD3 zeta signaling domain or region as described in U.S. Pat. No.: 7,446,190 or U.S. Pat. No. 8,911,993. For example, in some embodiments, the intracellular signaling domain or region comprises the sequence of amino acids as set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In some aspects, the spacer contains only a hinge region of an IgG, such as only a hinge of IgG4 or IgG1, such as the hinge only spacer set forth in SEQ ID NO: 1. In other embodiments, the spacer is or contains an Ig hinge, e.g., an IgG4-derived hinge, optionally linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 4. In some embodiments, the spacer is an Ig hinge, e.g., an IgG4 hinge, linked to a C_{H}3 domain only, such as set forth in SEQ ID NO: 3. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6 or 17, or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 6 or 17. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 7 or 16, or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 7 or 16.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

2. T Cell Receptors (TCRs)

In some embodiments, engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable α and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or Cβ, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or α and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naive Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the genetically engineered antigen receptors include recombinant T cell receptors (TCRs) and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified by a skilled artisan. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using computer prediction models known to those of skill in the art. In some embodiments, for predicting MHC class I binding sites, such models include, but are not limited to, ProPredl (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known to those of skill in the art. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007)

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable a domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known to those of skill in the art, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRa variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- wherein P is proline, G is glycine and S is serine (SEQ ID NO: 22). In some embodiments, the linker has the sequence GSADDAKKDAAKKDGKS (SEQ ID NO: 23)

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10⁻⁵ and 10⁻¹² M and all individual values and ranges therein. In some embodiments, the target antigen is an MHC-peptide complex or ligand.

In some embodiments, nucleic acid or nucleic acids encoding a TCR, such as a and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λX,G10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-C1, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other promoters known to a skilled artisan also are contemplated.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

3. Chimeric Auto-Antibody Receptors (CAARs)

In some embodiments, the recombinant receptor is a chimeric autoantibody receptor (CAAR). In some embodiments, the CAAR is specific for an autoantibody. In some embodiments, a cell expressing the CAAR, such as a T cell engineered to express a CAAR, can be used to specifically bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM). In some embodiments, the intracellular signaling region comprises a secondary or costimulatory signaling region (secondary intracellular signaling regions).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

4. Multi-Targeting

In some embodiments, the cells and methods include multi-targeting strategies, such as expression of two or more genetically engineered receptors on the cell, each recognizing the same of a different antigen and typically each including a different intracellular signaling component. Such multi-targeting strategies are described, for example, in International Patent Application Publication No: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-κB and/or AP-1, and/or induction of gene expression of factors such as cytokines, proliferation, and/or survival.

In some embodiments, the first and/or second receptor includes intracellular signaling domains or regions of costimulatory receptors such as CD28, CD137 (4-1 BB), OX40, and/or ICOS. In some embodiments, the first and second receptors include an intracellular signaling domain of a costimulatory receptor that are different. In one embodiment, the first receptor contains a CD28 costimulatory signaling region and the second receptor contain a 4-1BB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

B. Cells and Preparation of Cells for Genetic Engineering

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻ CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application Publication Number WO2009/072003, or US Patent Application Publication Number US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood.1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., International Patent Application Publication Number WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al.(2012) J Immunother. 35(9): 651-660, Terakura et al. (2012) Blood.1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

C. Vectors and Methods for Genetic Engineering

Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 April 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in International Patent Application Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the subject to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some embodiments, a single promoter may direct expression of an RNA that contains, in a single open reading frame (ORF), two or three genes (e.g. encoding the molecule involved in modulating a metabolic pathway and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No. 20070116690.

### III. COMPOSITIONS AND FORMULATIONS

In some embodiments, the cell therapy is provided as a composition or formulation, such as a pharmaceutical composition or formulation. Such compositions can be used in accord with the provided methods, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the T cell therapy, such as engineered T cells (e.g. CAR T cells), are formulated with a pharmaceutically acceptable carrier. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being prevented or treated with the cells, including one or more active ingredients where the activities are complementary to the cells and/or the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contain cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

### IV. TREATMENT AND METHODS

In some embodiments, the provided methods are associated with the administration of a cell therapy, such as for the treatment of diseases or conditions including various tumors. The methods involve administering engineered cells expressing recombinant receptors designed to recognize and/or specifically bind to molecules associated with the disease or condition and result in a response, such as an immune response against such molecules upon binding to such molecules. The receptors may include chimeric receptors, e.g., chimeric antigen receptors (CARs), and other transgenic antigen receptors including transgenic T cell receptors (TCRs), including any as described herein. In some embodiments, the provided methods are followed by a method of re-expanding the recombinant immune cells in vivo in the subject, such as by disrupting an area in a subject in which the engineered cells are present or likely to be present or were present or were likely to be present. In some embodiments, the area can be an area containing antigen-expressing cells recognized by the engineered cells, such as a lesion, e.g. a tumor, or a microenvironment of the lesion, e.g. tumor microenvironment.

In some embodiments, a dose of cells expressing a recombinant receptor are administered to a subject to treat or prevent diseases, conditions, and disorders, including cancers. In some embodiments, the cells, populations, and compositions are administered to a subject or patient having the particular disease or condition to be treated, e.g., via adoptive cell therapy, such as adoptive T cell therapy. In some embodiments, cells and compositions, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the chimeric antigen receptor or transgenic TCR specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, and parasitic disease, and autoimmune and inflammatory diseases. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma. In some embodiments, the subject has acute-lymphoblastic leukemia (ALL). In some embodiments, the subject has non-Hodgkin's lymphoma.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is or includes an antigen selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acetylcholine receptor, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erbB2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Ra), IL-13 receptor alpha 2 (IL-13Ra2), kinase insert domain receptor (kdr), kappa light chain, L1 cell adhesion molecule (L1CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen targeted by the receptor is or includes CD20, CD19, CD22, ROR1, CD45, CD21, CD5, CD33, Igkappa, Iglambda, CD79a, CD79b or CD30. In some embodiments, the antigen is or inludes a pathogen-specific antigen or a pathogen-expressed antigen. In some embodiments, the antigen is a viral antigen (such as a viral antigen from HIV, HCV, HBV, etc.), bacterial antigens, and/or parasitic antigens

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent. In some cases, such therapeutic agent is not the same as the agent used in the provided methods for disrupting the lesion.

In some embodiments, the methods comprise administration of a chemotherapeutic agent, e.g., a conditioning chemotherapeutic agent, for example, to reduce tumor burden prior to the administration.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 1 1 1 (1995), and U.S. Pat. No. 5,087,616.

A. Dosing

The pharmaceutical composition in some embodiments contains the cells in amounts effective to treat or prevent the disease or condition, such as a therapeutically effective or prophylactically effective amount. In some embodiments, the composition includes the cells in an amount effective to reduce burden of the disease or condition.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the first dose.

The term “split dose” refers to a dose that is split so that it is administered over more than one day. This type of dosing is encompassed by the present methods and is considered to be a single dose.

Thus, the dose in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the first dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio.

In some embodiments, one or more consecutive or subsequent dose of cells can be administered to the subject. In some embodiments, the consecutive or subsequent dose of cells is administered greater than or greater than about 7 days, 14 days, 21 days, 28 days or 35 days after initiation of administration of the first dose of cells. The consecutive or subsequent dose of cells can be more than, approximately the same as, or less than the first dose. In some embodiments, administration of the T cell therapy, such as administration of the first and/or second dose of cells, can be repeated.

In some embodiments, a dose of cells is administered to subjects in accord with the provided methods. In some embodiments, the size or timing of the doses is determined as a function of the particular disease or condition in the subject. It is within the level of a skilled artisan to empirically determine the size or timing of the doses for a particular disease. Dosages may vary depending on attributes particular to the disease or disorder and/or patient and/or other treatments.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸ total such cells, or the range between any two of the foregoing values.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3⁺/CAR+ or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷7.5×10⁷, 1×10⁸, or 5×10⁸ total recombinant receptor-expressing CD8+ T cells.

In some embodiments, the cell therapy comprises administration of a dose comprising a number of cell from or from about 1×10⁵ to 1×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 1×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+recombinant receptor-expressing cells, each inclusive. In some embodiments, the cell therapy comprises administration of a dose comprising a number of cell from or from about 1×10⁵ to 1×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

B. Disrupting and/or Treatment

In connection with administering a dose of the genetically engineered cells, such as recombinant receptor-expressing cells, e.g. CAR+ T cells, any method for disrupting an area, e.g. a lesion, in which the cells are present or likely to be present and/or effecting a treatment that includes one or more of a physical or mechanical manipulation of a lesion or portion thereof, radiation or administration of an immunomodulatory agent can be employed, such as methods described in Section B. In some embodiments, the disrupting and/or treating is carried out after administering the genetically engineered cells. In some embodiments, the disrupting and/or treating is carried out greater than or greater than about 1 week, 2 weeks, 3 weeks, 4, weeks, 5 weeks, 6 weeks, 7 weeks, 8 weeks, 1 month, 2 months, 3 months, 4 months, 5 months, 6 months, 1 year, 2 years or more after administering the genetically engineered cells (e.g. CAR-T cells).

In some embodiments, the treatment and/or disruption is performed at a time after the subject exhibit a partial response (PR) to the genetically engineered cells and/or after the subject does not respond to the genetically engineered cells within a certain time, e.g. 14-28 days, in order to improve response outcome. In certain embodiments, the treatment and/or disruption is performed at a time after the subject exhibits a partial response. In certain embodiments, the disrupting is carried out greater than or greater than about 1 week, 2 weeks, 3 weeks, 4, weeks, 5 weeks, 6 weeks, 7 weeks, 8 weeks, 1 month, 2 months, 3 months, 4 months, 5 months, 6 months, 1 year, 2 years or more after the subject exhibits a PR. In certain embodiments, the disrupting is carried out greater than or greater than about 1 week, 2 weeks, 3 weeks, 4, weeks, 5 weeks, 6 weeks, 7 weeks, 8 weeks, 1 month, 2 months, 3 months, 4 months, 5 months, 6 months, 1 year, 2 years or more after the subject exhibits a PR. In some embodiments, a complete response (CR) to the treatment is observed by the provided methods. In some embodiments, the subject has not previously achieved remission, such as a CR, to a prior therapy or to the genetically engineered cells, such as the recombinant receptor-expressing cells, e.g. CAR+ T cells.

In some embodiments, at or immediately prior to the time of the treatment and/or disruption, the subject has relapsed following remission in response to the administration of the genetically engineered cells. In some embodiments, relapse occurs at a time (e.g. within 1 months, 2 months, 3 months, 4 months, 5 months, 6 months, 1 year or more) following a complete response (CR) or PR to administration of the genetically engineered cells, such as recombinant receptor-expressing cells, e.g. CAR+ T cells. In some embodiments, the treatment and/or disruption is carried out within or within about 12 hours, 1 day, 2 days, 3 days, 4 days, 5 days, 6 days or one week after relapse or after relapse is detected or observed. In certain embodiments, the treatment and/or disruption is carried out within or within about 12 hours, 1 day, 2 days, 3 days, 4 days, 5 days, 6 days or one week after the subject has been determined to, or is suspected of experiencing a relapse.

In some embodiments, the treatment and/or disruption is carried out at a time when the number of engineered cells detectable in the blood from the subject is decreased compared to in the subject at a preceding time point after administration of the engineered cells. In some embodiments, the treatment and/or disruption is carried out at a time when the number of cells of the T cell therapy detectable in the blood is less than or less than about 1.5-fold, 2-fold, 3-fold, 4-fold, 5-fold, 10-fold, 50-fold or 100-fold or less the peak or maximum number of the cells of the T cell therapy detectable in the blood of the subject after initiation of administration of the T cell therapy; and/or at a time after a peak or maximum level of the cells of the T cell therapy are detectable in the blood of the subject, the number of cells of the T cell therapy detectable in the blood from the subject is less than less than 10%, less than 5%, less than 1% or less than 0.1% of total peripheral blood mononuclear cells (PBMCs) in the blood of the subject. In some embodiments, the treatment and/or disruption is carried out at a time when the number of cells of the T cell therapy detectable in the blood is or is about 10%, 20%, 30%, 40%, 50%, 60%, 70%, 80%, 90%, 95%, 98%, 99%, or 99.9% less than the peak or maximum number of the cells of the T cell therapy detectable in the blood of the subject after initiation of administration of the T cell therapy.

In some embodiments, the treatment and/or disruption is carried out at a time at which there is (i) less than at or about 10 engineered cells per microliter, (ii) less than or less than about 20%, 30%, 40% or 50% of the total number of peripheral blood mononuclear cells (PBMCs), (iii) less than or less than about 1×10⁵ engineered cells; or (iv) less than or less than about 5,000 copies of recombinant receptor-encoding DNA per micrograms DNA.

In some embodiments, the disrupting and/or treating is performed as part of a treatment regimen involving a single treatment, procedure, or manipulation. In particular embodiments, the disrupting and/or treating is performed as part of a treatment regimen involving more than one treatment, procedure, or manipulation. In certain embodiments, the treatment and/or disruption is performed with a treatment regimen involving multiple treatments over a treatment span of about an hour, about 6 hours, about 12 hours, about 24 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, or about 6 months. In some embodiments, the treatment and/or disruption is performed with multiple treatments over a treatment span of between about 1 minute and about 1 hour, between 1 hour and 12 hours, between about 12 hours and about 24 hours, between about 1 day and about 2 days, between about 1 day and about 5 days, between about 1 day and about 7 days, between about 1 week and about 4 weeks, between about 1 month and about 2 months in length. In some embodiments, the multiple treatments are preformed hourly, daily, every other day, every two days, every three days, every four days, every five days, every six days, once a week, twice a week, three times a week, four times a week, five times a week, six times a week, seven times a week, eight times a week, nine times a week, ten times a week, eleven times a week, twelve time a week, thirteen times a week, fourteen times a week, once a month, twice a month, three times a month, four times a month, five times a month, six times a month, seven times a month, eight times a month, nine times a month, ten times a month, eleven times a month, twelve times a month, thirteen times a month, fourteen times a month, once every two months, of once every three months over the treatment span.

In certain embodiments, the area (e.g. tissue, organ, mass or lesion areas of the subject or a region or portion thereof) is treated and/or disrupted by a treatment regimen involving multiple treatments (or procedures or manipulations) during a treatment cycle. A treatment cycle is a course of treatment that is repeated on a regular schedule. In some embodiments, a treatment cycle can comprise several days of treatment followed by several days of rest (i.e. a drug holiday). For example, a treatment may be performed daily for three weeks, followed by a week of no treatment, in a 28 day treatment cycle, or a treatment may be performed five times a week for the first three weeks, followed by a week with no treatments. In particular embodiments, a treatment is performed to treat and/or disrupt a lesion over one or more treatment cycles. A treatment cycle can be at least two, at least three, at least four, at least five, at least six, at least seven, at least 14, at least 21, at least 28, at least 48, or at least 96 days or more. In one embodiment, a treatment cycle is 28 days. In various embodiments, the treatment cycle is determined by a health care professional based on conditions and needs of the subject. In some embodiments, a treatment is performed on at least one day, at least two days, at least three days, at least four days, at least five days, at least six days, at least seven days, at least eight days, at least nine days, at least ten days, at least eleven days, at least twelve days, at least 13 days, at least 14 days, at least 21 days, or all 28 days of a 28 day treatment cycle.

In some embodiments, the area (e.g. tissue, organ, mass or lesion areas of the subject or a region or portion thereof) is treated and/or disrupted with a mechanical treatment and/or disruption, e.g., a biopsy. In certain embodiments, the lesion is mechanically treated and/or disrupted with a single treatment, procedure, or manipulation. In particular embodiments, the mechanical treatment and/or disruption comprises more than one treatment, procedure, or manipulation, e.g., a biopsy. In some embodiments, more than one lesion is treated and/or disrupted in a subject.

In particular embodiments, the area (e.g. tissue, organ, mass or lesion areas of the subject or a region or portion thereof) is treated and/or disrupted with a thermotherapy, e.g., a cryotherapy or a hyperthermic therapy. Thermotherapy can be administered according to any schedule, dose, or method known to one of skill in the art to be effective treatment and/or disruption of a lesion. In some embodiments, the lesion is treated and/or disrupted by a single treatment of a thermotherapy. In particular embodiments, the lesion is treated and/or disrupted with more than treatment of a thermotherapy. In certain embodiments, the thermotherapy can be cryoablation therapy. In some embodiments, the thermotherapy can be hyperthermic therapy. In some embodiments, the thermotherapy is a therapy that elevates the temperature of the tumor higher than in hyperthermic therapy.

In some embodiments, the lesion is treated and/or disrupted by irradiation and/or with a radiation therapy. Dosing is based on the International Unit known Gray (Gy, also expressed as cGy where 100 cGy=1 Gy) and the dose delivered during one treatment session is known as a fraction. For example, a typical dosing schedule for superficial radiation therapy (SRT) might be a total dose of 4,500 cGy (45 Gy) delivered in 300 cGy doses for a total of 15 fractions. Radiation treatments may be delivered over several weeks, with fractions given on certain days, e.g., Monday through Friday. Alternate dosing schedules are also used in clinical practice, which include 2 to 3 fractions per week (i.e., Monday, Wednesday, Friday schedule). Dosing for a patient is determined by a skilled clinician including with the aid of skilled technician, e.g., a radiation physicist, and is based on the size of the lesion, and the age and health of the subject.

In certain embodiments, the area (e.g. tissue, organ, mass or lesion areas of the subject or a region or portion thereof) is treated and/or disrupted by one or more treatments with radiation. In particular embodiments, the lesion is treated and/or disrupted with more than one treatment or dose of radiation, and the total dose is about 5 Gy, about 10 Gy, about 15 Gy, about 20 Gy, about 25 Gy, about 30 Gy, about 35 Gy, about 40 Gy, about 41 Gy, about 42 Gy, about 43 Gy, about 44 Gy, about 45 Gy, about 46 Gy, about 47 Gy, about 48 Gy, 49 Gy, about 50 Gy, about 51 Gy, about 52 Gy, about 53 Gy, about 54 Gy, about 55 Gy, about 56 Gy, about 57 Gy, about 58 Gy, about 59 Gy, about 60 Gy, about 61 Gy, about 62 Gy, about 63 Gy, about 64 Gy, about 65 Gy, about 70 Gy, about 80 Gy, about 90 Gy, or about 100 Gy. In some embodiments, the total dose is between about 0.01 Gy and about 1 Gy, between about 1 Gy and about 30 Gy, between about 1 Gy and about 15 Gy, between about 15 Gy and about 30 Gy, between about 30 Gy to about 90 Gy, about 30 Gy to about 45 Gy, between about 40 Gy and about 70 Gy, or between about 45 Gy to about 60 Gy.

In some embodiments, the lesion is treated and/or disrupted by two or more fractional treatments with radiation. In certain embodiments, the fractional dose is about 100 cGy, about 200 cGy, about 300 cGy, about 400 cGy, about 500 cGy, about 600 cGy, about 700 cGy, about 800 cGy, about 900 cGy, about 1 Gy, about 2 Gy, about 3 Gy, about 4 Gy, or about 5 Gy. In particular embodiments, the fractional dose is between about 10 cGy and about 100 cGy, between about 100 cGy and about 500 cGy, between about 500 cGy and about 1 Gy, or between about 1 Gy and about 5 Gy.

In some embodiments, the lesion is treated and/or disrupted by a single radiation treatment. In certain embodiments, the single dose is about 100 cGy, about 200 cGy, about 300 cGy, about 400 cGy, about 500 cGy, about 600 cGy, about 700 cGy, about 800 cGy, about 900 cGy, about 1 Gy, about 2 Gy, about 3 Gy, about 4 Gy, or about 5 Gy. In particular embodiments, the single dose is between about 10 cGy and about 100 cGy, between about 100 cGy and about 500 cGy, between about 500 cGy and about 1 Gy, or between about 1 Gy and about 5 Gy.

In certain embodiments, the lesion is treated and/or disrupted with external-beam radiation therapy (EBT). To treat and/or disrupt a lesion, EBT can be administered according to any schedule, dose, or method known to one of skill in the art to be effective in the treatment or amelioration of a hyperproliferative disorder, without limitation. In some embodiments, the lesion is treated and/or disrupted by administering EBT in a dose that is less than what is understood by one of skill in the art to be effective for treatment or amelioration of a hyperproliferative disorder. In general, external-beam radiation therapy comprises irradiating a defined volume within a subject with a high energy beam, thereby causing cell death within that volume. In some embodiments the irradiated volume contains the lesion to be treated and/or disrupted, and preferably contains as little healthy and/or non-lesion tissue as possible. In certain embodiments, the irradiated volume contains most or all of lesion. In some embodiments, the methods of administering and apparatuses and compositions useful for external-beam radiation therapy can be found in U.S. Pat. Nos. 6,449,336, 6,398,710, 6,393,096, 6,335,961, 6,307,914, 6,256,591, 6,245,005, 6,038,283, 6,001,054, 5,802,136, 5,596,619, and 5,528,652.

In some embodiments, the lesion is treated and/or disrupted with brachytherapy. In particular embodiments, brachytherapy can be administered according to any schedule, dose, or method known to one of skill in the art to be effective in the treatment or amelioration of a hyperproliferative disorder to treat and/or disrupt the lesion. In certain embodiments, the brachytherapy can be administered according to a schedule, dose, or method known to one of skill in the art to be less than what is effective in the treatment or amelioration of a hyperproliferative disorder. In general, brachytherapy comprises insertion of radioactive sources into the body of a subject to be treated for cancer, such as inside the tumor itself, such that the tumor is maximally exposed to the radioactive source, and minimizing the exposure of healthy tissue. Representative radioisotopes that can be administered in brachytherapy include, but are not limited to, phosphorus 32, cobalt 60, palladium 103, ruthenium 106, iodine 125, cesium 137, indium 192, xenon 133, radium 226, californium 252, or gold 198. Methods of administering and apparatuses and compositions useful for brachytherapy are described in Mazeron et al, Sem. Rad. One. 12:95-108 (2002), Kovacs J. Contemp. Brachytherapy. 6(4):404-416 (2015), and U.S. Patent Nos. 6,319,189, 6,179,766, 6,168,777, 6,149,889, and 5,611,767.

In some embodiments, one or more treatments, e.g., administrations of a pharmaceutical agent, such as therapeutic agent, are performed to treat and/or disrupt a lesion. In certain embodiments, the treatment comprises administering a dose of a pharmaceutical agent, such as any as described, e.g. immunomodulatory agent or compound. In particular embodiments, the pharmaceutical agent is administered once to treat and/or disrupt the lesion. In certain embodiments the pharmaceutical agent is administered more than once to treat and/or disrupt the lesion. In some embodiments, the pharmaceutical agent is administered at a dose in the range from about 0.0001 to about 100 mg/kg body weight, such as from about 0.0005 to about 50 mg/kg body weight, such as from about 0.001 to about 10 mg/kg body weight, e.g. from about 0.01 to about 1 mg/kg body weight. In particular embodiments, the pharmaceutical agent is administered to the subject at a dose of between about 0.001 mg to about 100 mg, about 0.05 mg to about 50 mg, about 0.01 mg to about 1 mg, about 1 mg to about 20 mg, or about 5 mg to about 15 mg. In some embodiments, the pharmaceutical agent is administered systemically. In certain embodiments, the agent is administered locally to the lesion. In particular embodiments, the pharmaceutical agent is administered orally, topically, sublingually, intravenously, subcutaneously, enterally, parenterally, by inhalation, and/or by injection.

In some embodiments, the pharmaceutical agent is a chemotherapeutic agent. In some embodiments, chemotherapeutic agents may be administered at a dose or doses that are recognized by those of skill in the art to be effective for the treatment of a hyperproliferative disorder. In certain embodiments, chemotherapeutic agents may be administered at doses lower than those used in the art that are recognized to be effective for the treatment of a hyperproliferative disorder. In some embodiments, a single dose of a chemotherapeutic agent is administered to treat and/or disrupt a lesion. In certain embodiments, more than one dose of a chemotherapeutic agent is administered to a subject over a treatment span, e.g. one or more treatment cycles, to treat and/or disrupt the lesion. In particular embodiments, the amount of treatments or administrations of the chemotherapeutic agent is a number that is recognized by those of skill in the art to be effective for the treatment of a hyperproliferative disorder. In some embodiments, the amount of treatments is less than a number that is recognized by those of skill in the art to be effective for the treatment of a hyperproliferative disorder.

In certain embodiments, the pharmaceutical agent is an immunomodulatory agent, e.g., a checkpoint inhibitor. In certain embodiments, immunomodulatory agents may be administered at a dose or doses that are recognized by those of skill in the art to be effective for the treatment of a hyperproliferative disorder. In certain embodiments, immunomodulatory agents may be administered at doses lower than those used in the art that are recognized to be effective for the treatment of a hyperproliferative disorder. In certain embodiments, a single dose of an immunomodulatory agent is administered to treat and/or disrupt a lesion. In some embodiments, more than one dose of an immunomodulatory agent is administered to a subject over a treatment span, e.g. one or more treatment cycles, to treat and/or disrupt the lesion. In particular embodiments, the amount of treatments or administrations of the immunomodulatory agent is a number that is recognized by those of skill in the art to be effective for the treatment of a hyperproliferative disorder. In some embodiments, the amount of treatments is less than a number that is recognized by those of skill in the art to be effective for the treatment of a hyperproliferative disorder.

In certain embodiments, the pharmaceutical agent is lenalidomide or a thalidomide derivative. In particular embodiments the pharmaceutical agent is lenalidomide. In some embodiments, lenalidomide or a thalidomide derivative is administered at a dosage of from about 1 mg to about 20 mg, e.g., from about 1 mg to about 10 mg, from about 2.5 mg to about 7.5 mg, from about 5 mg to about 15 mg, such as about 5 mg, 10 mg, 15 mg or 20 mg. In some embodiments, lenalidomide is administered at a dose of from about 10 μg/kg to 5 mg/kg, e.g., about 100 μg/kg to about 2 mg/kg, about 200 μg/kg to about 1 mg/kg, about 400 μg/kg to about 600 μg/kg, such as about 500 μg/kg. In particular embodiments, the dose of lenalidomide is or is about 10 mg. In certain embodiments, a lesion is treated and/or disrupted by administering a single dose of lenalidomide to the subject. In particular embodiments, a lesion is treated and/or disrupted by administering multiple doses of lenalidomide to the subject. In particular embodiments, the multiple doses of lenalidomide are administered over one or more treatment cycles. In some embodiments, the treatment cycles comprise a drug holiday. In certain embodiments, the lenalidomide is administered once daily for 14 days over a 21 day treatment cycle. In certain embodiments, the lenalidomide is administered once daily for 21 days over a 28 day treatment cycle.

In some embodiments, the treatment and/or disruption is repeated one or more times, such as by effecting one or more subsequent treatment and/or disruption after a prior or previous treatment and/or disruption. In some embodiments, the subsequent or repeated treatment and/or disruption is performed after the genetically engineered cells have expanded in the subject or been observed to have expanded after the preceding treatment and/or disruption and prior to the subsequent treatment and/or disruption. In some cases, the subsequent treatment and/or disruption is performed at a time wherein at or immediately prior to the time of the subsequent treatment and/or disruption, the subject is in remission. In some examples, the subsequent treatment and/or disruption is performed at a time wherein at or immediately prior to the time, the number of genetically engineered cells detectable in the blood is reduced or is not detectable. n some cases, the subsequent treatment and/or disruption is performed at a time wherein at or immediately prior to the time of the subsequent treatment and/or disruption, the number of genetically engineered cells detectable in a fluid or tissue or sample, optionally the blood, from the subject is decreased compared to a preceding time point after initiation of the preceding treatment and/or disruption. In some cases, the subsequent treatment and/or disruption is performed at a time wherein at or immediately prior to the time of the subsequent treatment and/or disruption, the number of cells of the genetically engineered cells detectable in a fluid or tissue or sample, optionally the blood, from the subject, is decreased by or more than 1.5-fold, 2.0-fold, 3.0-fold, 4.0-fold, 5.0-fold, 10-fold or more as compared to the peak or maximum number of the genetically engineered cells detectable or detected in the blood of the subject after initiation of the preceding treatment and/or disruption and/or compared to the level at a time point within 1, 2, 3, 4, 5, 6, 7, 8, 9, 10, 11, 12, 13, or 14 or 28 days following initiation of the preceding treatment and/or disruption.

In some embodiments, the method includes effecting a subsequent treatment and/or disruption, optionally after the subject has relapsed following response after the preceding treatment and/or disruption and/or has not achieved a complete response after the preceding treatment and/or disruption. In some cases, the subject had responded to the genetically engineered cells after the preceding treatment and/or disruption(s) and has subsequently ceased to respond and/or relapsed prior to the subsequent treatment and/or disruption. In some aspects, the genetically engineered cells have expanded in the subject or been observed to have expanded after the preceding treatment and/or disruption(s) and prior to the subsequent treatment and/or disruption.

C. Monitoring Expansion of Cells

In some embodiments, the method includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells used for the immunotherapy, e.g. T cell therapy, before or after administering the cell therapy provided herein.

In some embodiments, the presence and/or amount of cells expressing the recombinant receptor (e.g., CAR-expressing cells administered for T cell based therapy) in the subject following the administration of the T cells and before, during and/or after the administration of the therapy is detected. In particular embodiments, the presence and/or amount of cells expressing the recombinant receptor in the subject following the administration of the T cells and before, during and/or after the disrupting is detected. In some aspects, the presence and/or amount of cells, such as to monitor persistence, is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some cases, the presence of engineered cells, e.g. recombinant receptor-expressing cells, can be detected or monitored in other biological samples, such as organ or tissue samples (e.g. disease site, e.g. tumor sample) of the subject.

In some aspects, quantitative PCR (qPCR) is used to assess the quantity of cells expressing the recombinant receptor (e.g., CAR-expressing cells administered for T cell based therapy) in the blood or serum or organ or tissue sample (e.g., disease site, e.g., tumor sample) of the subject. In some cases, methods for assessing the presence or amount of cells expressing the recombinant receptor may include drawing peripheral blood (or other biological sample) from subjects that have been administered engineered cells, and determining the number or ratio of the engineered cells in the peripheral blood or biological sample. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (a CAR or TCR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the cells are detected in the subject at or at least at 4, 14, 15, 27, or 28 days following the administration of the T cells, e.g., CAR-expressing T cells. In some aspects, the cells are detected at or at least at 2, 4, or 6 weeks following, or 3, 6, or 12, 18, or 24, or 30 or 36 months, or 1, 2, 3, 4, 5, or more years, following the administration of the T cells, e.g., CAR-expressing T cells. In certain embodiments, the cells are detected in the subject at or at least at 1, 2, 3, 4, 5, 6, 7, 8, 9, 10, 11, 12, 13, 14, 15, 16, 17, 18, 19, 20, 21, 22, 23, 24, 25, 26, 27, or 28 days following the disrupting of the area in the subject, such as a tissue, organ, mass or lesion area of the subject or a region or portion thereof. In some embodiments, the cells are detected at or at least at 2, 4, or 6 weeks following, or 3, 6, or 12, 18, or 24, or 30 or 36 months, or 1, 2, 3, 4, 5, or more years, following the treatment and/or disruption of the area in the subject, such as a tissue, organ, mass or lesion area of the subject or a region or portion thereof.

In some embodiments, the presence and/or amount of cells is detected after an amount of time after an area in the subject, such as a tissue, organ, mass or lesion area of the subject or a region or portion thereof, has been treated and/or disrupted with more than one treatment, procedure, or manipulation over a treatment span, and the amount of time is measured from the start of first treatment, procedure, or manipulation. In particular embodiments, the presence and/or amount of cells is detected after an amount of time after an area in the subject, such as a tissue, organ, mass or lesion area of the subject or a region or portion thereof, has been treated and/or disrupted with more than one treatment, procedure, or manipulation over a treatment span, and the amount of time is measured from the end of final treatment, procedure, or manipulation.

The exposure, e.g., number of cells, e.g. T cells administered for T cell therapy, indicative of expansion and/or persistence, may be stated in terms of maximum numbers of the cells to which the subject is exposed, duration of detectable cells or cells above a certain number or percentage, area under the curve for number of cells over time, and/or combinations thereof and indicators thereof. Such outcomes may be assessed using known methods, such as qPCR to detect copy number of nucleic acid encoding the recombinant receptor compared to total amount of nucleic acid or DNA in the particular sample, e.g., blood, serum, plasma or tissue, such as a tumor sample, and/or flow cytometric assays detecting cells expressing the receptor generally using antibodies specific for the receptors. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor.

In some aspects, increased exposure of the subject to the cells includes increased expansion of the cells. In some embodiments, the receptor expressing cells, e.g. CAR-expressing cells, expand in the subject following administration of the T cells, e.g., CAR-expressing T cells. In particular embodiments, disrupting the area (e.g. a tissue, organ, mass or lesion areas of the subject or a region or portion thereof) results in increased exposure to the receptor expressing cells e.g., the CAR-expressing cells, such as increased expansion of the cells in the subject compared to the expansion of the cells immediately prior to the disrupting or compared to the peak expansion of the cells in the subject prior to the disrupting of the area (e.g. a tissue, organ, mass or lesion areas of the subject or a region or portion thereof).

In some aspects, the method, e.g. disrupting the area (e.g. a tissue, organ, mass or lesion areas of the subject or a region or portion thereof), results in high in vivo proliferation of the administered cells, for example, as measured by flow cytometry. In some aspects, high peak proportions of the cells are detected. For example, in some embodiments, at a peak or maximum level following the administration of the T cells, e.g., CAR-expressing T cells, in the blood or disease-site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some embodiments, the method e.g., disrupting the area (e.g. a tissue, organ, mass or lesion areas of the subject or a region or portion thereof), results in a maximum concentration, in the blood or serum or other bodily fluid or organ or tissue of the subject, of at least 100, 500, 1000, 1500, 2000, 5000, 10,000 or 15,000 copies of or nucleic acid encoding the receptor, e.g., the CAR, per microgram of DNA, or at least 0.1, 0.2, 0.3, 0.4, 0.5, 0.6, 0.7, 0.8, or 0.9 receptor-expressing, e.g., CAR,-expressing cells per total number of peripheral blood mononuclear cells (PBMCs), total number of mononuclear cells, total number of T cells, or total number of microliters. In some embodiments, the cells expressing the receptor are detected as at least 10, 20, 30, 40, 50, or 60% of total PBMCs in the blood of the subject, and/or at such a level for at least 1, 2, 3, 4, 5, 6, 7, 8, 9, 10, 11, 12, 24, 36, 48, or 52 weeks following the initiation of administration of the T cells, e.g., CAR-expressing T cells or for 1, 2, 3, 4, or 5, or more years following such administration.

In some aspects, the method results in at least a 2-fold, at least a 4-fold, at least a 10-fold, or at least a 20-fold increase in copies of nucleic acid encoding the recombinant receptor, e.g., CAR, per microgram of DNA, e.g., in the serum, plasma, blood or tissue, e.g., tumor sample, of the subject.

In some embodiments, cells expressing the receptor are detectable in the serum, plasma, blood or tissue, e.g., a tumor or lesion sample, of the subject, e.g., by a specified method, such as qPCR or flow cytometry-based detection method, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more days following administration of the T cells, e.g., CAR-expressing T cells , for at least at or about 2, 3, 4, 5, 6, 7, 8, 9, 10, 11, 12, 13, 14, 15, 16, 17, 18, 19, 20, 21, 22, 23, or 24 or more weeks following the administration of the T cells, e.g., CAR-expressing T cells. In particular embodiments, cells expressing the receptor are detectable in the serum, plasma, blood or tissue of the subject at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more days following the treatment and/or disruption of the area (e.g. a tissue, organ, mass or lesion areas of the subject or a region or portion thereof), for at least at or about 2, 3, 4, 5, 6, 7, 8, 9, 10, 11, 12, 13, 14, 15, 16, 17, 18, 19, 20, 21, 22, 23, or 24 or more weeks following the treatment and/or disruption of the lesion.

In some aspects, at least about 1×10², at least about 1×10³, at least about 1×10⁴, at least about 1×10⁵, or at least about 1×10⁶ or at least about 5×10⁶ or at least about 1×10⁷ or at least about 5×10⁷ or at least about 1×10⁸ recombinant receptor-expressing, e.g., CAR-expressing cells, and/or at least 10, 25, 50, 100, 200, 300, 400, or 500, or 1000 receptor-expressing cells per microliter, e.g., at least 10 per microliter, are detectable or are present in the subject or fluid, plasma, serum, tissue, or compartment thereof, such as in the blood, e.g., peripheral blood, or disease site, e.g., lesion tumor, thereof. In some embodiments, such a number or concentration of cells is detectable in the subject for at least about 20 days, at least about 40 days, or at least about 60 days, or at least about 3, 4, 5, 6, 7, 8, 9, 10, 11, or 12 months, or at least 2 or 3 years, following administration of the T cells, e.g., CAR-expressing T cells. In certain embodiments, such a number or concentration of cells is detectable in the subject for at least about 20 days, at least about 40 days, or at least about 60 days, or at least about 3, 4, 5, 6, 7, 8, 9, 10, 11, or 12 months, or at least 2 or 3 years, following treatment and/or disruption of the lesion. Such cell numbers may be as detected by flow cytometry-based or quantitative PCR-based methods and extrapolation to total cell numbers using known methods. See, e.g., Brentjens et al., Sci Transl Med. 2013 5(177), Park et al, Molecular Therapy 15(4):825-833 (2007), Savoldo et al., JCI 121(5):1822-1826 (2011), Davila et al., (2013) PLoS ONE 8(4):e61338, Davila et al., Oncoimmunology 1(9):1577-1583 (2012), Lamers, Blood 2011 117:72-82, Jensen et al., Biol Blood Marrow Transplant 2010 September; 16(9): 1245-1256, Brentjens et al., Blood 2011 118(18):4817-4828.

In some aspects, the copy number of nucleic acid encoding the recombinant receptor, e.g., vector copy number, per 100 cells, for example in the peripheral blood or bone marrow or other compartment, as measured by immunohistochemistry, PCR, and/or flow cytometry, is at least 0.01, at least 0.1, at least 1, or at least 10, at about 1 week, about 2 weeks, about 3 weeks, about 4 weeks, about 5 weeks, or at least about 6 weeks, or at least about 2, 3, 4, 5, 6, 7, 8. 9, 10, 11, or 12 months or at least 2 or 3 years following administration of the cells, e.g., CAR-expressing T cells. In some embodiments, the copy number of the vector expressing the receptor, e.g. CAR, per microgram of genomic DNA is at least 100, at least 1000, at least 5000, or at least 10,000, or at least 15,000 or at least 20,000 at a time about 1 week, about 2 weeks, about 3 weeks, or at least about 4 weeks following administration of the T cells, e.g., CAR-expressing T cells or at least 2, 3, 4, 5, 6, 7, 8, 9, 10, 11, or 12 months or at least 2 or 3 years following such administration.

In certain embodiments, the copy number of nucleic acid encoding the recombinant receptor, e.g., vector copy number, per 100 cells, as measured by immunohistochemistry, PCR, and/or flow cytometry, is at least 0.01, at least 0.1, at least 1, or at least 10, at about 1 week, about 2 weeks, about 3 weeks, about 4 weeks, about 5 weeks, or at least about 6 weeks, or at least about 2, 3, 4, 5, 6, 7, 8. 9, 10, 11, or 12 months or at least 2 or 3 years following the treatment and/or disruption of the lesion. In some embodiments, the copy number of the vector expressing the receptor, e.g. CAR, per microgram of genomic DNA is at least 100, at least 1000, at least 5000, or at least 10,000, or at least 15,000 or at least 20,000 at a time about 1 week, about 2 weeks, about 3 weeks, or at least about 4 weeks following the treatment and/or disruption of the lesion or at least 2, 3, 4, 5, 6, 7, 8, 9, 10, 11, or 12 months or at least 2 or 3 years following the treatment and/or disruption of the lesion.

In some aspects, the receptor, e.g. CAR, expressed by the cells, is detectable by quantitative PCR (qPCR) or by flow cytometry in the subject, plasma, serum, blood, tissue and/or disease site thereof, e.g., tumor site, at a time that is at least about 3 months, at least about 6 months, at least about 12 months, at least about 1 year, at least about 2 years, at least about 3 years, or more than 3 years, following the administration of the cells, e.g., following the initiation of the administration of the T cells. In particular embodiments, the receptor expressed by the cells is detectable in the subject, plasma, serum, blood, tissue and/or disease site thereof, e.g., lesion or tumor site, at a time that is at least about 3 months, at least about 6 months, at least about 12 months, at least about 1 year, at least about 2 years, at least about 3 years, or more than 3 years, following the treatment and/or disruption of the lesion. In some embodiments, the area under the curve (AUC) for concentration of receptor- (e.g., CAR-) expressing cells in a fluid, plasma, serum, blood, tissue, organ and/or disease site, e.g. tumor site, of the subject over time following the administration of the T cells, e.g., CAR-expressing T cells, is measured.

D. Response, Efficacy and Survival

In some embodiments, the administration effectively treats the subject despite the subject having become resistant to another therapy and/or having relapsed following administration of the genetically engineered cells, such as recombinant receptor-expressing cells, e.g. CAR+ T cells. In some embodiments, at least or about at least 50% of subjects, at least or about at least 60% of the subjects, at least or about at least 70% of the subjects, at least or about at least 80% of the subjects or at least or about at least 90% of the subjects treated according to the method achieve complete remission (CR) and/or achieve an objective response (OR).

In some embodiments, the subjects treated according to the provided method e.g., involving disrupting the area or lesion following administration of genetically engineered cells, achieve a more durable response, or achieves a more durable response on average in a plurality of subjects so treated, compared to methods involving administration of the genetically engineered cells, such as recombinant receptor-expressing cells, but not involving treatment and/or disruption of the area or lesion as described herein. In some cases, a measure of duration of response (DOR) includes the time from documentation of tumor response to disease progression. In some embodiments, the parameter for assessing response can include durable response, e.g., response that persists after a period of time after it is observed following initiation of therapy or after treatment and/or disruption of the area or lesion following initiation of the genetically engineered cells. In some embodiments, durable response is indicated by the response rate at approximately 1, 2, 3, 4, 5, 6, 7, 8, 9, 10, 11, 12, 18 or 24 months after initiation of therapy or after treatment and/or disruption of the area or lesion following initiation of administration of the genetically engineered cells. In some embodiments, the response is durable for greater than 3 months, greater than 6 months, greater than 12 months, greater than 18 months, greater than 24 months, greater than 30 months, greater than 36 months or more. In some particular embodiments, the subjects treated according to the method achieve a durable response after the subject previously relapsed following remission in response to the administration of the genetically engineered cells.

In some aspects, response rates in subjects, such as subjects with CLL, are based on the International Workshop on Chronic Lymphocytic Leukemia (IWCLL) response criteria (Hallek, et al., Blood 2008, June 15; 111(12): 5446-5456). In some aspects, these criteria are described as follows: complete remission (CR), which in some aspects requires the absence of peripheral blood clonal lymphocytes by immunophenotyping, absence of lymphadenopathy, absence of hepatomegaly or splenomegaly, absence of constitutional symptoms and satisfactory blood counts; complete remission with incomplete marrow recovery (CRi), which in some aspects is described as CR above, but without normal blood counts; partial remission (PR), which in some aspects is described as ≥50% fall in lymphocyte count, ≥50% reduction in lymphadenopathy or ≥50% reduction in liver or spleen, together with improvement in peripheral blood counts; progressive disease (PD), which in some aspects is described as ≥50% rise in lymphocyte count to >5×109/L, ≥50% increase in lymphadenopathy, ≥50% increase in liver or spleen size, Richter's transformation, or new cytopenias due to CLL; and stable disease, which in some aspects is described as not meeting criteria for CR, CRi, PR or PD.

In some embodiments, the subjects exhibits a CR or OR if, within 1 month of the administration of the dose of cells, lymph nodes in the subject are less than at or about 20 mm in size, less than at or about 10 mm in size or less than at or about 10 mm in size. In particular embodiments, the subjects exhibits a CR or OR if, within 1 month of the administration of the treatment and/or disruption of the lesion, lymph nodes in the subject are less than at or about 20 mm in size, less than at or about 10 mm in size or less than at or about 10 mm in size.

In some embodiments, an index clone of the CLL is not detected in the bone marrow of the subject (or in the bone marrow of greater than 50%, 60%, 70%, 80%, 90% or more of the subjects treated according to the methods. In some embodiments, an index clone of the CLL is assessed by IgH deep sequencing. In some embodiments, the index clone is not detected at a time that is at or about or at least at or about 1, 2, 3, 4, 5, 6, 12, 18 or 24 months following the administration of the cells.

In some aspects, response assessment utilizes any of clinical, hematologic, and/or molecular methods. In some respects, response is assessed using the Lugano criteria (Cheson et al., (2014) JCO 32(27):3059-3067; Johnson et al., (2015) Radiology 2:323-338; Cheson, B.D. (2015) Chin Clin Oncol 4(1):5). In some aspects, response assessment utilizes any of clinical, hematologic, and/or molecular methods. In some aspects, response assessed using the Lugano criteria involves the use of positron emission tomography (PET)—computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake≤mediastinum; 3, uptake>mediastinum but≤liver; 4, uptake moderately>liver; 5, uptake markedly higher than liver and/or new lesions; X, new areas of uptake unlikely to be related to lymphoma.

In some aspects, a complete response as described using the Lugano criteria involves a complete metabolic response and a complete radiologic response at various measureable sites. In some aspects, these sites include lymph nodes and extralymphatic sites, wherein a CR is described as a score of 1, 2, or 3 with or without a residual mass on the 5-point scale, when PET-CT is used. In some aspects, in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. In some aspects, response is assessed in the lymph nodes using CT, wherein a CR is described as no extralymphatic sites of disease and target nodes/nodal masses must regress to ≤1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology, which if indeterminate should be IHC negative. Further sites may include assessment of organ enlargement, which should regress to normal. In some aspects, nonmeasured lesions and new lesions are assessed, which in the case of CR should be absent. (Cheson et al., (2014) JCO 32(27):3059-3067; Johnson et al., (2015) Radiology 2:323-338; Cheson, B. D. (2015) Chin Clin Oncol 4(1):5).

In some aspects, a partial response (PR) as described using the Lugano criteria involves a partial metabolic and/or radiological response at various measureable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease. In some aspects, response is assessed in the lymph nodes using CT, wherein a PR is described as ≥50% decrease in SPD of up to 6 target measureable nodes and extranodal sites. If a lesion is too small to measure on CT, 5 mm×5 mm is assigned as as the default value; if the lesion is no longer visible, the value is 0 mm×0 mm; for a node >5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, nonmeasured lesions and new lesions are assessed, which in the case of PR should be absent/normal, regressed, but no increase. No response/stable disease (SD) or progressive disease (PD) can also be measured using PET-CT and/or CT based assessments. (Cheson et al., (2014) JCO 32(27):3059-3067; Johnson et al., (2015) Radiology 2:323-338; Cheson, B. D. (2015) Chin Clin Oncol 4(1):5).

In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some aspects, the RECIST criteria is used to determine objective tumor response; in some aspects, in solid tumors. (Eisenhauer et al., European Journal of Cancer 45 (2009) 228-247.) In some aspects, the RECIST criteria is used to determine objective tumor response for target lesions. In some respects, a complete response as determined using RECIST criteria is described as the disappearance of all target lesions and any pathological lymph nodes (whether target or non-target) must have reduction in short axis to <10 mm. In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

In some aspects, the administration or treatment in accord with the provided methods generally reduces or prevents the expansion or burden of the disease or condition in the subject. For example, where the disease or condition is a tumor, the methods generally reduce tumor size, bulk, metastasis, percentage of blasts in the bone marrow or molecularly detectable cancer and/or improve prognosis or survival or other symptom associated with tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood or bone marrow in the context of certain hematological malignancies. Disease burden can include, in some embodiments, the mass of a tumor, the number or extent of metastases and/or the percentage of blast cells present in the bone marrow.

In some embodiments, a subject has leukemia. The extent of disease burden can be determined by assessment of residual leukemia in blood or bone marrow.

In some embodiments, a subject exhibits morphologic disease if there are greater than or equal to 5% blasts in the bone marrow, for example, as detected by light microscopy, such as greater than or equal to 10% blasts in the bone marrow, greater than or equal to 20% blasts in the bone marrow, greater than or equal to 30% blasts in the bone marrow, greater than or equal to 40% blasts in the bone marrow or greater than or equal to 50% blasts in the bone marrow. In some embodiments, a subject exhibits complete or clinical remission if there are less than 5% blasts in the bone marrow.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable cancer. In some embodiments, molecularly detectable cancer can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify cancer cell based on leukemia-specific immunophenotypes. In some embodiments, molecular detection of cancer can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In some aspects, the disease or condition persists following administration of the first dose and/or administration of the first dose is not sufficient to eradicate the disease or condition in the subject. In some embodiments, response to treatment, such as resolution of disease and/or remission, is observed in accord with the provided methods after disrupting the area or lesion as described.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative dosing regimen, such as one in which the subject receives one or more alternative therapeutic agents and/or one in which the subject does not receive treatment and/or disruption of an area in a subject in which the engineered cells are present or likely to be present or were present or were likely to be present in accord with the provided methods. In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified.

In some embodiments, the event-free survival rate or overall survival rate of the subject is improved by the methods, as compared with other methods, for example, methods in which the subject receives one or more alternative therapeutic agents and/or one in which the subject does not receive treatment and/or disruption of an area in a subject in which the engineered cells are present or likely to be present or were present or were likely to be present in accord with the provided methods. For example, in some embodiments, event-free survival rate or probability for subjects treated by the methods at 6 months following the dose is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods, for example, methods in which the subject receives one or more alternative therapeutic agents and/or one in which the subject does not receive treatment and/or disruption of an area in a subject in which the engineered cells are present or likely to be present or were present or were likely to be present in accord with the provided methods. For example, in some embodiments, the probability of relapse at 6 months following the first dose is less than about 80%, less than about 70%, less than about 60%, less than about 50%, less than about 40%, less than about 30%, less than about 20%, or less than about 10%.

### V. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, a “subject” is a mammal, such as a human or other animal, and typically is human. In some embodiments, the subject, e.g., patient, to whom the immunomodulatory polypeptides, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or engineered cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering the immunomodulatory polypeptides, engineered cells, or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g. : Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New.Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, the term about refers to ±25%, ±20%, ±10%, ±5,

As used herein, a composition refers to any mixture of two or more products, substances, or compounds, including cells. It may be a solution, a suspension, liquid, powder, a paste, aqueous, non-aqueous or any combination thereof.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### VI. EXEMPLARY EMBODIMENTS

Among the provided embodiments are:

1. A method for expanding genetically engineered cells, comprising effecting disruption of an area in a subject in which the genetically engineered cells are present or likely to be present or were present or were likely to be present, said subject having previously received administration of the genetically engineered cells for treating a disease or condition, wherein the method results in expansion of the genetically engineered cells in the subject, in the area, and/or in a tissue or organ or fluid of the subject and/or in an increased number of the genetically engineered cells in the area, tissue or organ or fluid.

2. The method of embodiment 1, wherein the method does not comprise a subsequent administration of genetically engineered cells and/or the expansion is achieved without such a subsequent administration of the genetically engineered cells.

3. A method of treatment, comprising administering a treatment regimen to a subject, wherein the subject has previously been administered genetically engineered cells for treating a disease or condition, wherein the method results in expansion of the genetically engineered cells in the subject, in the area, and/or in a tissue or organ or fluid of the subject and/or in an increased number of the genetically engineered cells in the area, tissue or organ or fluid.

4. The method of embodiment 4, wherein the treatment regimen comprises a disruption of an area in a subject in which the engineered cells are present are suspected of being present or having been present, or likely to be present.

5. The method of embodiment 3 or 4, wherein the treatment regimen and/or the method does not comprise a subsequent administration of genetically engineered cells or of the genetically engineered cells and/or the expansion is achieved without such a subsequent administration.

6. The method of any one of embodiments 3-5, wherein the treatment regimen is administered at a sub-therapeutic dose and/or derives its therapeutic effect via expansion of the genetically engineered cells.

7. The method of any of embodiments 1, 2 and 4-6, wherein the area is or comprises a lesion or portion thereof.

8. The method of embodiment 7, wherein the lesion is a tumor.

9. The method of embodiment 8, wherein the tumor is a primary or secondary tumor.

10. The method of embodiment any of embodiments 1, 2 and 4-6, wherein the area is or comprises bone marrow tissue.

11. The method of any of embodiments 1, 2 and 4-10, wherein at or immediately prior to the time of the disruption, the subject has relapsed following after response, optionally after remission, and/or did not respond to the administration of the genetically engineered cells.

12. The method of any one of embodiments 1-11, wherein the subject has relapsed after response to, and/or did not respond to, the previous administration of genetically engineered cells.

13. The method of any one of embodiments 1-12, wherein the subject had responded to the genetically engineered cells and has subsequently ceased to respond and/or relapsed prior to the disruption.

14. The method of any one of embodiments 1-13, wherein the genetically engineered cells have previously expanded in the subject or been observed to have expanded prior to the disruption.

15. The method of any of embodiments 1-14, wherein at or immediately prior to the time of the disruption:

the subject is in remission;

the number of genetically engineered cells detectable in the blood is reduced or is not detectable;

the number of genetically engineered cells detectable in a fluid or tissue or sample, optionally the blood, from the subject is decreased compared to a preceding time point after administration of the genetically engineered cells; and/or

the number of cells of the genetically engineered cells detectable in a fluid or tissue or sample, optionally the blood, from the subject, is decreased by or more than 1.5-fold, 2.0-fold, 3.0-fold, 4.0-fold, 5.0-fold, 10-fold or more as compared to the peak or maximum number of the genetically engineered cells detectable or detected in the blood of the subject after initiation of administration of the genetically engineered cells and/or compared to the level at a time point within 1, 2, 3, 4, 5, 6, 7, 8, 9, 10, 11, 12, 13, or 14 or 28 days following the administration of the genetically engineered cells.

16. The method of any of embodiments 1-15, wherein the disruption is carried out at, at about, or greater than, or greater than about 2 weeks, 1 month, 2 months, 3 months, 4 months, 5 months, 6 months, 1 year or more after initiation of administration of the genetically engineered cells or after the last dose of the genetically engineered cells.

17. The method of any of embodiments 1-16, wherein the disruption directly or indirectly modulates an activity or function of the genetically engineered T cells in vivo in the subject.

18. The method of any of embodiments 1-17, wherein the disruption comprises one or more of administration of an immunomodulatory agent, radiation or a physical or mechanical manipulation of the area or lesion.

19. The method of any of embodiments 1-18, wherein the disruption comprises administration of an immunomodulatory agent.

20. The method of embodiment 19, wherein the immunomodulatory agent is or comprises an immune-inhibitory molecule, is or comprises an immune checkpoint molecule or member of an immune checkpoint pathway and/or is or comprises a modulator of an immune checkpoint molecule or pathway.

21. The method of embodiment 20, wherein the immune checkpoint molecule or pathway is or comprises PD-1, PD-L1, PD-L2, CTLA-4, LAG-3, TIM3, VISTA, an adenosine receptor, CD73, CD39, adenosine 2A Receptor (A2AR), or adenosine or a pathway involving any of the foregoing.

22. The method of any of embodiments 1-20, wherein the immunomodulatory agent is BY55, MSB0010718C, ipilimumab, Daclizumab, Bevacizumab, Basiliximab, Ipilimumab, Nivolumab, pembrolizumab, MPDL3280A, Pidilizumab, MK-3475, BMS-936559, Atezolizumab, tremelimumab, IMP321, BMS-986016, LAG525, urelumab, PF-05082566, TRX518, MK-4166, dacetuzumab, lucatumumab, SEA-CD40, CP-870, CP-893, MEDI6469, MEDI6383, MOXR0916, AMP-224, Avelumab, MEDI4736, PDR001, rHIgM12B7, Ulocuplumab, BKT140, Vα rlilumab, ARGX-110, MGA271, lirilumab, IPH2201, ARGX-115, Emactuzumab, CC-90002 and MNRP1685A or an antibody-binding fragment thereof.

23. The method of any of embodiments 1-22, wherein the immunomodulatory agent is an anti-PD-L1 antibody.

24. The method of embodiment 1-23, wherein the anti-PD-L1 antibody is MEDI14736, MDPL3280A, BMS-936559, LY3300054, atezolizumab or avelumab or is an antigen-binding fragment thereof.

25. The method of embodiment 19, wherein the immunomodulatory agent is thalidomide or is a derivative or analogue of thalidomide.

26. The method of embodiment 19 or 25, wherein the immunomodulatory agent is lenalidomide or pomalidomide, avadomide, a stereoisomer of lenalidomide, pomalidomide, avadomide or a pharmaceutically acceptable salt, solvate, hydrate, co-crystal, clathrate, or polymorph thereof.

27. The method of any of embodiments 19, 25 and 26, wherein the immunomodulatory agent is lenalidomide, a stereoisomer of lenalidomide or a pharmaceutically acceptable salt, solvate, hydrate, co-crystal, clathrate, or polymorph thereof.

28. The method of any of embodiments 1-27, wherein after the relapse and prior to the disruption, the subject has not been administered an exogenous or recombinant agent for treating the disease or condition or for modulating the activity of the genetically engineered cells.

29. The method of any of embodiments 1-19 and 28, wherein the disruption comprises radiation.

30. The method of any of embodiments 1-19 and 28, wherein the disruption comprises a physical or mechanical manipulation of the area or lesion, optionally comprises probing, poking or penetrating the area or lesion.

31. The method of embodiment 30, wherein the physical or mechanical manipulation comprises a biopsy.

32. The method of embodiment 31, wherein the biopsy is carried out by a needle or a trocar.

33. The method of embodiment 31 or embodiment 32, wherein the biopsy comprises an incisional biopsy.

34. The method of any of embodiments 1-34, wherein the methods results in expansion of the genetically engineered cells or an increase in the number of the genetically engineered cells compared to at the time just prior to the disruption.

35. The method of any of embodiments 1-34, wherein expansion of the genetically engineered cells occurs within or within about 24 hours, 48 hours, 96 hours, 7 days, 14 days or 28 days after the disruption.

36. The method of any of embodiments 1-35, wherein:

the expansion results in greater than or greater than about 1.5-fold, 2.0-fold, 5.0-fold, 10-fold, 100-fold, 200-fold, or more genetically engineered cells detectable in the blood compared to just prior to the disruption; or

the expansion results in greater than or greater than about 1.5-fold, 2.0-fold, 5.0-fold, 10-fold, 100-fold, 200-fold, or more genetically engineered cells detectable in the blood compared to the prior peak levels of engineered cells in the blood prior to the disruption.

37. The method of any of embodiments 1-36, wherein the number of genetically engineered cells detectable in the blood at a time after the disruption is:

increased (e.g. increase by 1.5-fold, 2-fold, 3-fold, 4-fold, 5-fold, 10-fold or more decreased) compared to the number of the genetically engineered cells at a preceding time point before the disruption;

more than 1.5-fold 2-fold, 3-fold, 4-fold, 5-fold, 10-fold, 50-fold, 100-fold or more than the peak or maximum number of the genetically engineered cells detectable in the blood of the subject before the disruption;

more than or about more than 10%, 9%, 8%, 7%, 6%, 5%, 4%, 3%, 2%, 1%, 0.5%, 0.2% or 0.1% of the genetically engineered cells are detectable in the blood at a time after a peak of maximum level of such cells has been detected in the blood.

38. The method of any of embodiments 1-37, wherein the engineered cells express a recombinant receptor that specifically binds to an antigen associated with the disease or condition or expressed in cells of the environment or of the area, optionally the lesion.

39. The method of any of embodiments 1-38, wherein the disease or condition is a tumor or a cancer.

40. The method of any of embodiments 1-39, wherein the disease or condition is a leukemia or lymphoma.

41. The method of any of embodiments 1-40, wherein the disease or condition is a non-Hodgkin lymphoma (NHL), an acute lymphoblastic leukemia (ALL) or a chronic lymphocytic leukemia (CLL).

42. The method of any of embodiments 1-41, wherein the recombinant receptor is a T cell receptor or a functional non-T cell receptor.

43. The method of any of embodiments 1-42, wherein the recombinant receptor is a chimeric antigen receptor (CAR).

44. The method of embodiment 43, wherein the CAR comprises an extracellular antigen-recognition domain that specifically binds to the antigen and an intracellular signaling domain comprising an ITAM.

45. The method of any of embodiments 38-44, wherein the antigen is CD19.

46. The method of embodiment 44 or embodiment 45, wherein the intracellular signaling domain comprises an intracellular domain of a CD3-zeta (CD3) chain.

47. The method of any of embodiments 43-46, wherein the CAR further comprises a costimulatory signaling region.

48. The method of embodiment 35, wherein the costimulatory signaling domain comprises a signaling domain of CD28 or 4-1BB.

49. The method of any of embodiments 1-48, wherein the engineered cells are CD4+ or CD8+ T cells.

50. The method of any of embodiments 1-49, wherein the T cell therapy comprises primary cells derived from a subject.

51. The method of any of any of embodiments 1-49, wherein the engineered cells are autologous to the subject.

52. The method of any of embodiments 1-49, wherein the engineered cells are allogeneic to the subject.

53. The method of any of embodiments 1-52, wherein the subject is a human. 54. The method of any of embodiments 1-53, wherein the dose of genetically engineered cells previously administered is from or from about 1×10⁵ to 1×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive.

55. The method of any of embodiments 1-54, wherein the dose of genetically engineered cells previously administered is no more than 1×10⁸ total recombinant receptor-expressing cells, total T cells, or total peripheral blood mononuclear cells (PBMCs), no more than 1×10⁷ total recombinant receptor-expressing cells, total T cells, or total peripheral blood mononuclear cells (PBMCs), no more than 0.5×10⁷ total recombinant receptor-expressing cells, total T cells, or total peripheral blood mononuclear cells (PBMCs), no more than 1×10⁶ total recombinant receptor-expressing cells, total T cells, or total peripheral blood mononuclear cells (PBMCs), no more than 0.5×10⁶ total recombinant receptor-expressing cells, total T cells, or total peripheral blood mononuclear cells (PBMCs).

56. The method of any of embodiments 1-53, wherein the dose of genetically engineered cells previously administered is between about 0.25×10⁶ cells/kg body weight of the subject and 5×10⁶ cells/kg, 0.5×10⁶ cells/kg body weight of the subject and 3×10⁶ cells/kg, between about 0.75×10⁶ cells/kg and 2.5×10⁶ cells/kg or between about 1×10⁶ cells/kg and 2×10⁶ cells/kg, each inclusive.

57. The method of any of embodiments 1-56, wherein the dose of genetically engineered cells are administered in a single pharmaceutical composition comprising the cells or as a plurality of compositions together comprising the cells.

58. The method of any of embodiments 1-57, wherein the engineered cells administered is a split dose, wherein the cells of the dose are administered in a plurality of compositions, collectively comprising the cells of the dose, over a period of no more than three days.

59. The method of any of embodiments 1-58, wherein the method comprises effecting a subsequent disruption, optionally after the subject has relapsed following response after the preceding disruption and/or has not achieved a complete response after the preceding disruption.

60. The method of any embodiment 59, wherein the subject had responded to the genetically engineered cells after the preceding disruption and has subsequently ceased to respond and/or relapsed prior to the subsequent disruption.

61. The method of embodiment 59 or embodiment 60, wherein the genetically engineered cells have expanded in the subject or been observed to have expanded after the preceding disruption and prior to the subsequent disruption.

62. The method of any of embodiments 59-61, wherein at or immediately prior to the time of the subsequent disruption:

the subject is in remission;

the number of genetically engineered cells detectable in the blood is reduced or is not detectable;

the number of genetically engineered cells detectable in a fluid or tissue or sample, optionally the blood, from the subject is decreased compared to a preceding time point after initiation of the preceding disruption; and/or

the number of cells of the genetically engineered cells detectable in a fluid or tissue or sample, optionally the blood, from the subject, is decreased by or more than 1.5-fold, 2.0-fold, 3.0-fold, 4.0-fold, 5.0-fold, 10-fold or more as compared to the peak or maximum number of the genetically engineered cells detectable or detected in the blood of the subject after initiation of the preceding disruption and/or compared to the level at a time point within 1, 2, 3, 4, 5, 6, 7, 8, 9, 10, 11, 12, 13, or 14 or 28 days following initiation of the preceding disruption.

63. The method of any of embodiments 1-62, wherein the genetically engineered cells exhibits increased or prolonged expansion and/or persistence in the subject as compared to a method in which the genetically engineered cells are administered to the subject in the absence of the disruption.

64. The method of any of embodiments 1-63, wherein the method reduces tumor burden to a greater degree and/or for a greater period of time as compared to the reduction that would be observed with a comparable method in which the genetically engineered cells are administered to the subject in the absence of the disruption and/or in which the therapeutic regimen is administered or the disruption is effected in the absence of the genetically engineered cells, optionally at the same dose or dosing schedule.

### VII. EXAMPLES

The following examples are included for illustrative purposes only and are not intended to limit the scope of the invention.

### Example 1

### Administration of Anti-CD19 CAR-Expressing Cells to Subjects

Twenty eight subjects with relapsed or refractory (R/R) non-Hodgkin lymphoma (NHL) were administered autologous T cells expressing an anti-CD19 chimeric antigen receptor (CAR). Subject demographics and baseline characteristics are set forth in Table 1. The CAR contained an anti-CD19 scFv derived from a murine antibody, an immunoglobulin-derived spacer, a transmembrane domain derived from CD28, a costimulatory region derived from 4-1BB, and a CD3-zeta intracellular signaling domain. To generate the autologous CAR-expressing T cells, T cells were isolated by immunoaffinity-based enrichment from leukapheresis samples from individual subjects, activated and transduced with a viral vector encoding an anti-CD19 CAR, followed by expansion. Cells generally were administered to subjects at a target CAR⁺ CD4⁺ T cell to CAR⁺ CD8⁺ T cell ratio of approximately 1:1.

TABLE 1
Demographics and Baseline Characteristics

N = 28

Characteristic

| | | |
|---|---|---|
| Median Age, years (range) | 63 | (37-79) |
| ≥70 years, n (%) | 6 | (21) |
| Male/Female, n (%) | 19/9 | (68/32) |

B-NHL Subtype, n(%)

| | | |
|---|---|---|
| DLBCL, NOS | 15 | (54) |
| Transformed DLBCL | 10 | (36) |
| Follicular, Grade 3B | 1 | (4) |
| MCL | 2 | (7) |

Disease Status, n (%)

| | | |
|---|---|---|
| Refractory* | 24 | (86) |
| Chemorefractory^{†} | 23 | (82) |

Baseline ECOG score, n (%)

| 0 | 14 | (50) |
|---|---|---|
| 1 | 10 | (36) |
| 2 | 4 | (14) |
| Prior Lines of Therapy | | |
| Median (range) | 4 | (1-8) |
| ≥5, n (%) | 7 | (25) |

Prior Hematopoietic Stem Cell Transplant, n (%)

| | | |
|---|---|---|
| Any HSCT | 16 | (57) |
| Allogeneic | 4 | (14) |
| Autologous | 13 | (46) |

*<CR to last therapy
^{†}SD or PD to last chemo-containing regimen or relapse <12 months after autologous SCT

Prior to administration of the CAR-expressing T cells (d=0), subjects were treated with 30 mg/m² fludarabine daily for 3 days and 300 mg/m² cyclophosphamide daily for 3 days. The cryopreserved cell compositions were thawed prior to intravenous administration. The therapeutic T cell dose was administered as a defined cell composition by administering a formulated CD4+ CAR+ cell population and a formulated CD8+ CAR+ population administered at a target ratio of approximately 1:1. At d=0, treatment of subjects was initiated, with a single-dose or two-dose schedule, at one of two dose levels (dose level 1 (DL-1) or dose level 2 (DL-2), by intravenous infusion. Each dose administered included 5×10⁷ (DL-1) or 1×10⁸ (DL-2) CAR-expressing T cells (target 1:1 CD4+:CD8+ ratio). Results in this example refer to those results observed up to and at a particular timepoint in an ongoing study, of specified group(s) of subjects.

The presence or absence of various treatment-emergent adverse events was assessed in subjects treated with various dose schedules of CAR-T cell therapy (Table 2). As shown in Table 3, no severe Cytokine Release Syndrome (sCRS) (Grade 3-5) was observed; Cytokine Release Syndrome (CRS) was observed in 36% (10/28) of the subjects. Grade 3-4 neurotoxicity was observed in 14% (4/28) of the subjects and 18% (5/28) of the subjects exhibited neurotoxicity of any grade. One subject was treated with tocilizumab and four patients received dexamethasone for early onset Grade 2 CRS or neurotoxicity. Six subjects received prophylactic anti-epileptics.

TABLE 2
Treatment-Emergent Adverse Events

| | DL1-S | | DL1-D | | DL2-S | | Total | |
|---|---|---|---|---|---|---|---|---|
| | N = 22 | | N = 3 | | N = 3 | | N = 28 | |
| Any TEAE | 21 | (96) | 3 | (100) | 3 | (100) | 27 | (96) |
| Any Grade 3-5* TEAE | 16 | (73) | 3 | (100) | 0 | | 19 | (68) |
| Any Related TEAE | 14 | (64) | 2 | (67) | 1 | (33) | 17 | (61) |

| | | | | | | | |
|---|---|---|---|---|---|---|---|
| Any Related Grade 3-5* | 4 | (18) | 1 | (33) | 0 | 5 | (18) |
| TEAE | | | | | | | |

All grade TEAEs reported in ≥15% patients Preferred term, n (%)

| Fatigue | 7 | (32) | 2 | (67) | 2 | (67) | 11 | (39) |
|---|---|---|---|---|---|---|---|---|
| Cytokine release | 8 | (36) | 2 | (67) | 0 | | 10 | (36) |
| syndrome | | | | | | | | |
| Decreased appetite | 6 | (27) | 1 | (33) | 1 | (33) | 8 | (29) |
| Constipation | 5 | (23) | 1 | (33) | 1 | (33) | 7 | (25) |
| Vomiting | 5 | (23) | 1 | (33) | 1 | (33) | 7 | (25) |
| Diarrhea | 5 | (23) | 1 | (33) | 0 | | 6 | (21) |
| Dizziness | 6 | (27) | 0 | | 0 | | 6 | (21) |
| Headache | 4 | (18) | 1 | (33) | 0 | | 5 | (18) |
| Hypertension | 4 | (18) | 1 | (33) | 0 | | 5 | (18) |
| Nausea | 3 | (14) | 1 | (33) | 1 | (33) | 5 | (18) |

| | | | | | | |
|---|---|---|---|---|---|---|
| Peripheral edema | 5 | (23) | 0 | 0 | 5 | (18) |

Lab abnormalities

| | | | | | | | | |
|---|---|---|---|---|---|---|---|---|
| Anemia | 16 | (73) | 1 | (33) | 1 | (33) | 18 | (64) |
| Neutropenia | 22 | (100) | 3 | (100) | 2 | (67) | 27 | (96) |
| Thrombocytopenia | 13 | (59) | 3 | (100) | 2 | (67) | 18 | (64) |

*1 Grade 5 respiratory failure, assessed as possibly related to CAR-T cell therapy, in a patient with MCL who progressed and started on a subsequent therapy

TABLE 3
Treatment-Emergent Adverse Events of Special Interest

| | DL1-S | DL1-D | DL2-S | Total | |
|---|---|---|---|---|---|
| Preferred Term, n (%) | N = 22 | N = 3 | N = 3 | N = 28 | |
| Cytokine Release Syndrome | 8 (36) | 2 (67) | 0 | 10 | (36) |
| (CRS), any | | | | | |
| Grade 3-4 | 0 | 0 | 0 | 0 | |
| Neurotoxicity, any* | 4 (18) | 1 (33) | 0 | 5 | (18) |
| Grade 3-4 | 3 (14) | 1 (33) | 0 | 4 | (14) |

*Includes: encephalopathy, confusional state, depressed level of consciousness, lethargy, or seizure

Subjects among the group were assessed for best overall response, observed over a period of up to a particular time-point in an ongoing study after the last CAR+ T cell infusion of single-dose of DL1. Results of overall responses are shown in Table 4. Of the 20 subjects that were treated with the single-dose of DL1 in the Diffuse Large B-Cell Lymphoma (DLBCL) cohort, an overall response rate (ORR) of 80% (16/20) was observed and 60% (12/20) of subjects showed evidence of complete remission (CR). 20% (4/20) of subjects showed evidence of partial response (PR) and 20% (4/20) of subjects showed evidence of progressive disease (PD). Of the subjects having been chemorefractory (having exhibited stable or progressive disease following last chemo-containing regimen or relapse less than 12 months after autologous SCT) prior to CAR+ T cell administration, the overall response rate was 83% (10 ORR, 7 CR, 3 PR, 2 PD, n=12). Among the subjects having been refractory (having exhibited less than complete remission following last treatment but not deemed chemorefractory), the overall response rate was 77% (13 ORR, 9 CR, 4 PR, 4PD, n=17).

TABLE 4
Best Overall Response

| | DLBCL Cohort, DL1 single-dose schedule | | |
|---|---|---|---|
| | All | Refractory* | Chemorefractory^{†} |
| | (n = 20) | (n = 17) | (n = 12) |
| ORR, n (%) | 16 (80) [56, 94] | 13 (77) [50, 93] | 10 (83) [52, 98] |
| [95% CI] | | | |
| CR, n (%) | 12 (60) [36, 81] | 9 (53) [28, 77] | 7 (58) [28, 85] |
| [95% CI] | | | |
| PR | 4 (20) | 4 (24) | 3 (25) |
| PD | 4 (20) | 4 (24) | 2 (17) |

*<CR to last therapy
^{†}SD or PD to last chemo-containing regimen or relapse <12 months after autologous SCT

Of three DLBCL subjects that at the time of assessment had been treated with two doses of DL-1, two exhibited partial response (PR) and 1 exhibited progressive disease (PD). Among 2 DLBCL subjects that at the time of assessment had been treated with a single-dose of DL-2, both subjects were observed to achieve CR. Among a MCL cohort with a total of two subjects treated at the time of assessment with single-dose of DL-1 1 PR and 1 PD were observed. Two subjects with double-hit, three subjects with triple-hit, and four subjects with double-expressor DLBCL were treated and all achieved a response (7 CR, 2 PR).

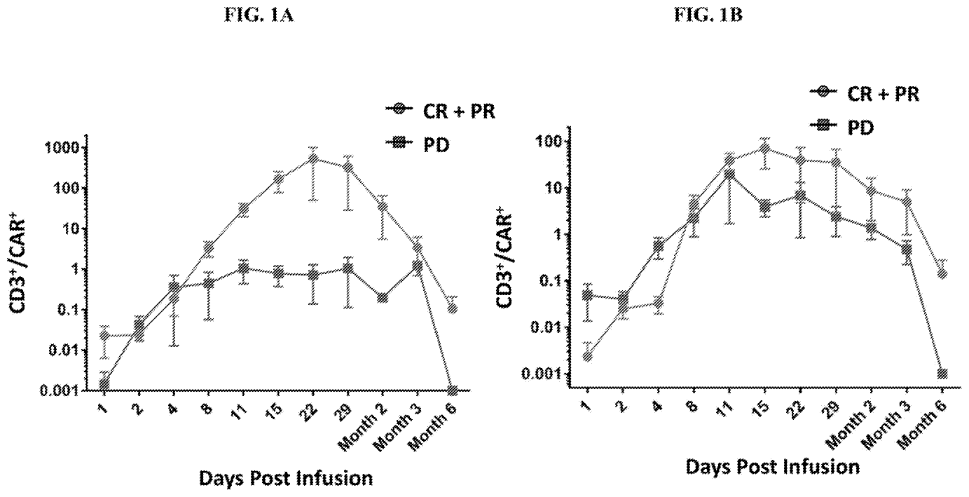

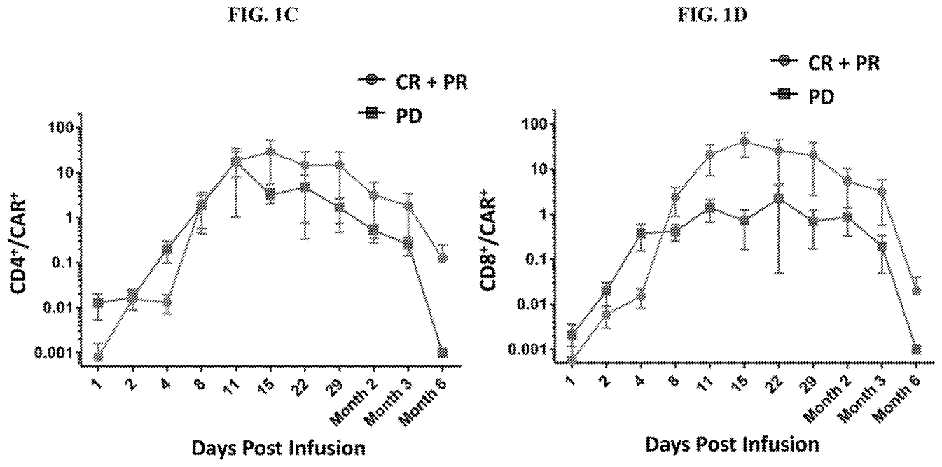

The number of CAR⁺ T cells in peripheral blood was determined at certain time points post-treatment by incubating cells with a transgene-specific reagent. The number of CD3⁺/CAR⁺ T cells in peripheral blood measured at certain time points post-infusion is shown for subjects grouped by best overall response in FIG. 1A. Higher peak CD3⁺/CAR⁺ T cells were observed in responders (CR/PR) than PD. FIG. 1B-D show levels of CD3⁺/CAR⁺ T cells, CD4⁺/CAR⁺ T, and CD8⁺/CAR⁺ T cell levels (cells/μL blood; mean±SEM) in subjects who achieved a response, grouped by durability of response, either continued response (CR/PR) or PD at 3 months. The Cₘₐₓ (CAR⁺ cells/μL blood) and area under the curve (AUC) for responders (CR/PR) and PD were determined and shown in Table 5. The results were consistent with a conclusion that durable responses correlated with higher CD3⁺/CAR⁺ T cell levels in the blood, over time and at peak expansion.

TABLE 5
Cₘₐₓ and AUC₀₋₂₈ Higher in Patients with CR/PR vs PD

| CD3 | | CD4 | | CD8 | |
|---|---|---|---|---|---|
| CR/PR | PD | CR/PR | PD | CR/PR | PD |
| (n = 16) | (n = 4) | (n = 16) | (n = 4) | (n = 16) | (n = 4) |

Cₘₐₓ (CAR⁺ cells/μL blood)

| | | | | | | |
|---|---|---|---|---|---|---|
| Mean (SD) | 612 (1919) | 2 (1) | 220 (754) | 1 (0.6) | 426 (1314) | 0.5 (0.5) |
| Median | 33 (1, 7726) | 1 (1, 3) | 8 (1, 3040) | 1 (0, 2) | 4 (0, 5238) | 0.3 (0, 1) |
| (Min, Max) | | | | | | |
| Q1, Q3 | 7, 123 | 0.7, 2 | 2, 46 | 0.6, 2 | 0.8, 104 | 0.1, 0.9 |

AUC₀₋₂₈

| | | | | | | |
|---|---|---|---|---|---|---|
| Mean (SD) | 5883 (18821) | 16 (13) | 2369 (8388) | 10 (7) | 3873 (11963) | 6 (6) |
| Median | 196 (11, 75773) | 14 (4, 31) | 47 (7, 33740) | 9 (3, 17) | 23 (1, 47834) | 4 (1, 14) |
| (Min, Max) | | | | | | |
| Q1, Q3 | 52, 781 | 5, 26 | 16, 261 | 4, 16 | 4, 761 | 1, 10 |

### Example 2

### Re-Expansion of Anti-CD19 CAR-Expressing Cells

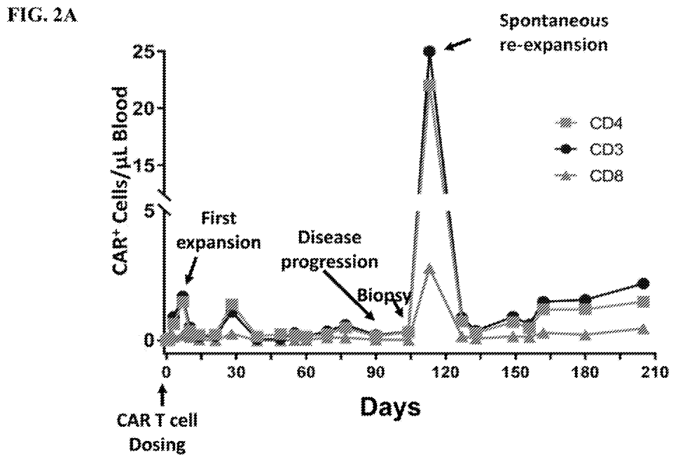

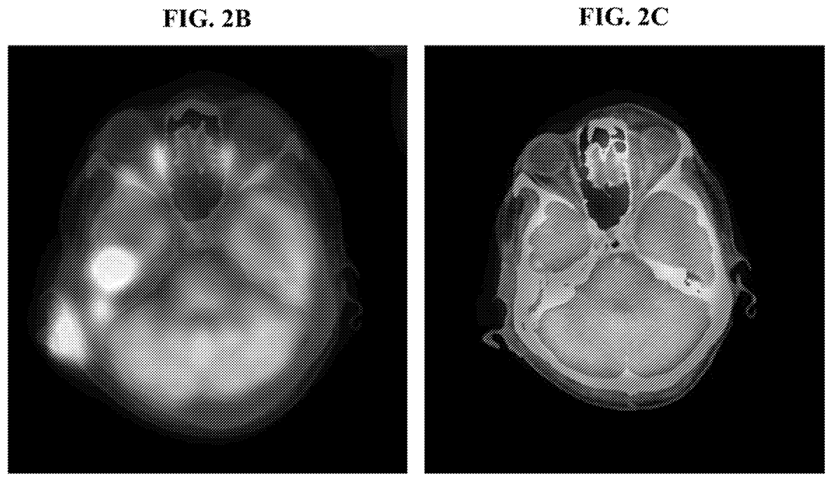

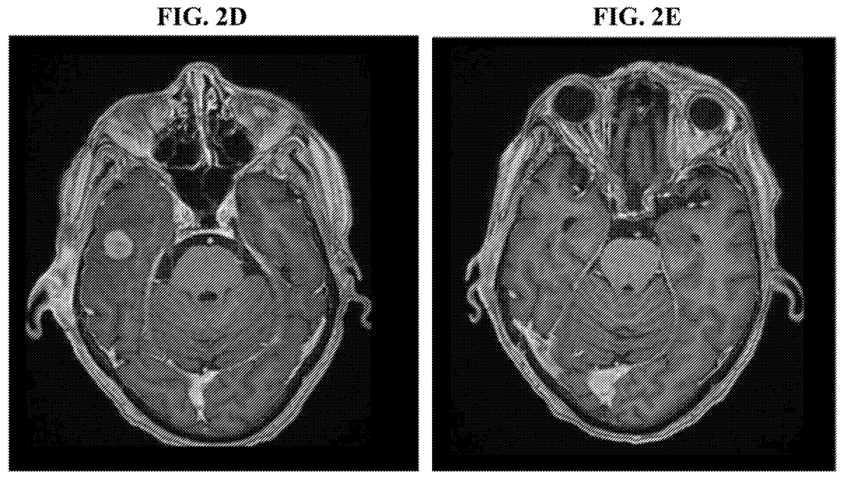

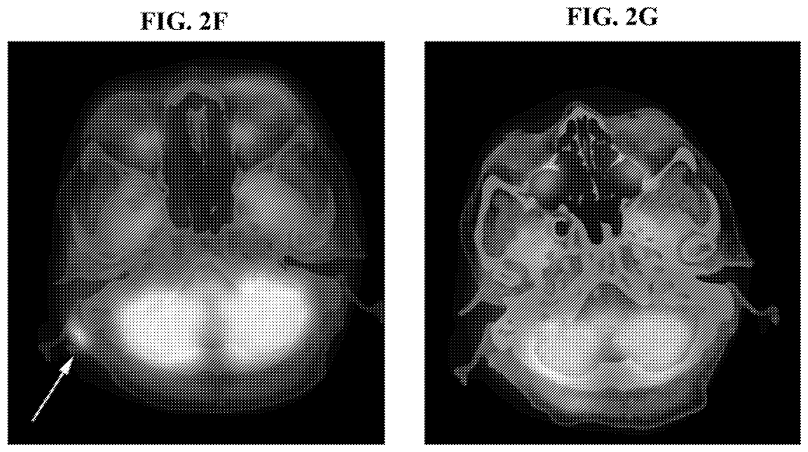

For one subject with chemorefractory transformed DLBCL (germinal center subtype with a BCL2 rearrangement and multiple copies of MYC and BCL6) who had been administered the CAR+ T cells in accordance with the DL-1 schedule as discussed in Example 1, the number of CD3⁺/CAR+, CD4⁺/CAR+, CD8+/CAR+ T cells in peripheral blood, measured at certain time points, are shown in FIG. 2A. The subject had previously been treated with, and was refractory to, five prior lines of therapy including dose-adjusted etoposide, doxorubicin, and cyclophosphamide with vincristine and prednisone plus rituximab (DA-EPOCH-R) and intermediate-intensity allogenic stem-cell transplantation from an 8/8 HLA-matched unrelated donor. Following allogeneic stem cell transplantation and prior to receiving CAR+ T cells, the subject showed 100% donor chimerism in all blood lineages, had ceased taking immunosuppressive therapy, and did not have graft versus host disease (GVHD). Prior to administration of CAR+ T cells, the subject had a periauricular mass and right-temporal lobe brain lesion observed by positron-emission tomography and computed tomography (PET-CT) (FIG. 2B) and confirmed by magnetic resonance imaging (MRI) (FIG. 2D).

After receiving anti-CD19 CAR-T cell treatment, the subject achieved CR 28 days post-infusion, as shown by PET-CT (FIG. 2C) and brain MRI (FIG. 2E), with no observed signs of neurotoxicity or CRS. Three months post-infusion of the CAR-T cells, relapse of the periauricular mass was noted in this subject (FIG. 2F), and an incisional biopsy was performed. As shown in FIG. 2A, following biopsy, the visible tumor receded with no further therapy. Pharmacokinetic analysis showed a marked re-expansion of the CAR-T cells in peripheral blood (to a level higher than initial expansion observed, with peak levels observed at about 113 days post-infusion), which coincided with tumor regression. The subject then went on to achieve a second CR, as confirmed by restaging PET-CT one month following the biopsy (FIG. 2G),and remained in CR at 6 months post CAR-T cell infusion. Further assessment of the subject showed that the CNS response was durable and the subject remained in CR at 12 months.

The results are consistent with a conclusion that re-expansion and activation of CAR+ T cells can be initiated in vivo following reduction or loss of functional or active CAR+ T cells and/or relapse following anti-tumor response to CAR-T cell therapy. Further, following re-expansion in vivo late after initial CAR+ T cell infusion, the CAR+ T cells are able to re-exert anti-tumor activity. This result supports that CAR+ T cell re-expansion and activation can be triggered in vivo and that methods of reactivating CAR+ T cells may further augment their efficacy.

### Example 3

### Administration of Anti-CD19 CAR-Expressing Cells to Subjects with Relapsed and Refractory Non-Hodgkin's Lymphoma (NHL)

A. Subjects and Treatment

Therapeutic CAR+ T cell compositions containing autologous T cells expressing a chimeric antigen-receptor (CAR) specific for CD19 were administered to subjects with B cell malignancies.

### Example 3.A.1

Results are described in this Example 3.A.1 for evaluation through a particular time-point (3.A.1) in an ongoing clinical study administering such therapy to patients with B cell Malignancies. Specifically, a cohort (full cohort) (at this time-point, fifty-five (55)) adult human subjects with relapsed or refractory (R/R) aggressive non-Hodgkin's lymphoma (NHL), including diffuse large B-cell lymphoma (DLBCL), de novo or transformed from indolent lymphoma (NOS), primary mediastinal large b-cell lymphoma (PMBCL), and follicular lymphoma grade 3b (FLG3B) after failure of 2 lines of therapy. Among the subjects treated were those having Eastern Cooperative Oncology Group (ECOG) scores of between 0 and 2 (median follow-up 3.2 months). The full cohort did not include subjects with mantle cell lymphoma (MCL). No subjects were excluded based on prior allogenic stem cell transplantation (SCT) secondary central nervous system (CNS) involvement or an ECOG score of 2, and there was no minimum absolute lymphocyte count (ALC) for apheresis required.

Outcomes were separately assessed for a core subset of subjects within the full cohort (subjects within the full cohort excluding those subjects with a poor performance status (ECOG 2), DLBCL transformed from marginal zone lymphomas (MZL) and/or chronic lymphocytic leukemia (CLL, Richter's) (core cohort)). At the time point in Example 3.A.1, outcomes for 44 subjects within this core cohort were separately assessed.

The demographics and baseline characteristics of the full and core cohort subjects, assessed at the timepoint in this example 3.A.1, are set forth in Table 6.

TABLE 6
Demographics and Baseline Characteristics

| | |
|---|---|
| FULL | CORE |
| N = 55 | N = 44 |

Characteristic

| | | | | |
|---|---|---|---|---|
| Median Age, years (range) | 61 | (29-82) | 61 | (29-82) |
| ≥65 years, n (%) | 22 | (40) | 17 | (39) |
| Male/Female, n (%) | 38/17 | (69/31) | 28/16 | (64/36) |
| Months from diagnosis, median (range) | 17 | (3-259) | 20 | (8-259) |

B-NHL Subtype, n (%)

| | | | | |
|---|---|---|---|---|
| DLBCL, NOS | 40 | (73) | 35 | (80) |
| Transformed DLBCL | 14 | (26) | 8 | (18) |
| Follicular, Grade 3B | 1 | (2) | 1 | (2) |

Molecular Subtype, n (% )

| | | | | |
|---|---|---|---|---|
| Double/triple hit | 15 | (27) | 12 | (27) |
| Double expressor | 6 | (11) | 4 | (9) |

Patient Characteristics, n (%)

| Chemorefractory^{†} | 42 | (76) | 34 | (77) |
|---|---|---|---|---|
| ECOG 0-1 | 48 | (87) | 44 | (100) |
| ECOG 2 | 7 | (13) | 0 | |
| Prior lines of therapy, median (range) | 3 | (1-11) | 3 | (1-8) |
| <5 lines of therapy | 44 | (80) | 37 | (84) |
| Any HSCT | 27 | (49) | 22 | (50) |
| Allogeneic | 4 | (7) | 3 | (7) |
| Autologous | 24 | (44) | 20 | (45) |

*SD or PD to last chemo-containing regimen or relapse <12 months after autologous SCT

The therapeutic T cell compositions administered had been generated by a process including immunoaffinity-based enrichment of CD4+ and CD8+ cells from leukapheresis samples from the individual subjects to be treated. Isolated CD4+ and CD8+ T cells were activated and transduced with a viral vector encoding an anti-CD19 CAR, followed by expansion and cryopreservation of the engineered cell populations. The CAR contained an anti-CD19 scFv derived from a murine antibody, an immunoglobulin-derived spacer, a transmembrane domain derived from CD28, a costimulatory region derived from 4-1BB, and a CD3-zeta intracellular signaling domain.

The cryopreserved cell compositions were thawed prior to intravenous administration. The therapeutic T cell dose was administered as a defined cell composition by admininstering a formulated CD4+ CAR+ cell population and a formulated CD8+CAR+population administered at a target ratio of approximately 1:1. Subjects were administered a single or double dose of CAR-expressing T cells (each single dose via separate infusions of CD4+ CAR-expressing T cells and CD8+CAR-expressing T cells, respectively) as follows: a single dose of dose level 1 (DL-1) containing 5×10⁷ total CAR-expressing T cells (n=30 for subjects assessed in Example 3.A.1), a double dose of DL1 in which each dose was administered approximately fourteen (14) days part (n=6 for subjects assessed in Example 3.A.1, including one subject that inadvertently received two DL2 doses via the two-dose schedule, due to a dosing error), or a single dose of dose level 2 (DL-2) containing 1×10⁸ (DL-2) total CAR-expressing T cells (n=18 for subjects assessed in Example 3.A.1). Beginning at three (3) days prior to CAR+ T cell infusion, subjects received a lymphodepleting chemotherapy with fludarabine (flu, 30 mg/m²) and cyclophosphamide (Cy, 300 mg/m²).

### Example 3.A.2

For example 3.A.2, at a subsequent point in time in the clinical study described in this Example 3 above, results were analyzed. At this analysis time point, 74 patients had been treated (51 male, 23 female). The subjects included sixty-bine (69) subjects in the full DLBCL cohort (including 67 DLBCL NOS (45 de novo, 14 transformed from FL, 8 transformed from CLL or MZL), 1 FL grade 3B, 1 PMBCL); and 5 subjects in the MCL cohort. Among subjects in the full (DLBCL) cohort, median age was 61 yrs (range 26, 82), median prior therapies was 3 (range 1, 12), 46 (67%) were chemorefractory, 32 (46%) had any prior transplant, and at least 16 (23%) patients had double/triple hit lymphoma. Forty-nine (49) subjects in the core cohort were assessed at this timepoint in 3.A.2.

B. Safety

The presence or absence of treatment-emergent adverse events (TEAE) following administration of the CAR-T cell therapy was assessed. Subjects also were assessed and monitored for neurotoxicity (neurological complications including symptoms of confusion, aphasia, encephalophathy, myoclonus seizures, convulsions, lethargy, and/or altered mental status), graded on a 1-5 scale, according to the National Cancer Institute—Common Toxicity Criteria (CTCAE) scale, version 4.03 (NCI-CTCAE v4.03). Common Toxicity Criteria (CTCAE) scale, version 4.03 (NCI-CTCAE v4.03). See Common Terminology for Adverse Events (CTCAE) Version 4, U.S. Department of Health and Human Services, Published: May 28, 2009 (v4.03: June 14, 2010); and Guido Cavaletti & Paola Marmiroli Nature Reviews Neurology 6, 657-666 (December 2010). Cytokine release syndrome (CRS) also was determined and monitored, graded based on severity. See Lee et al, Blood. 2014;124(2):188-95.

### Example 3.B.1

Example 3.B.1 describes results based on the analysis time-point in Example 3.A.1.

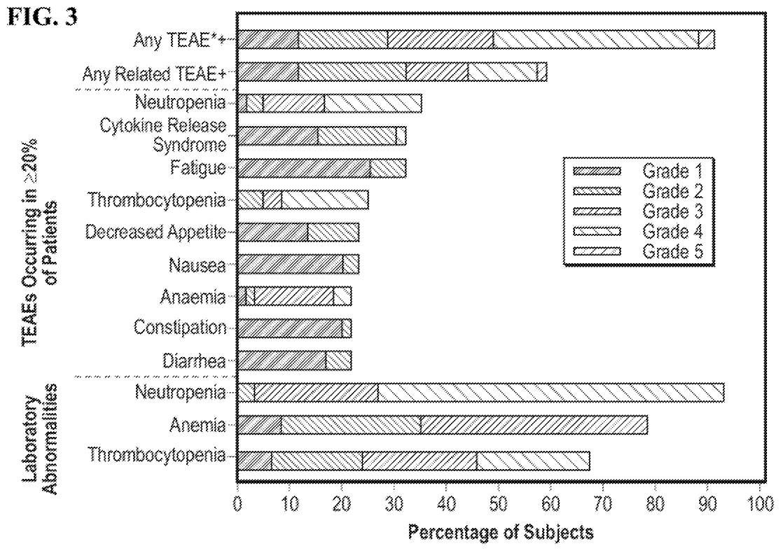

FIG. 3 depicts the percentage of such subjects who were observed to have experienced laboratory abnormalities and TEAEs, which occurred in >20% of subjects. In addition to the TEAEs shown in FIG. 3, the following event terms were observed at Grade 3-4 in ≥5% of patients: white blood cell count decreased (13.6%), encephalopathy (12%), hypertension (7%). Degree of toxicities observed were consistent between dose levels 1 and 2.

In 84% of the full cohort subjects in Example 3.B.1 analysis, severe (grade 3 or higher) cytokine release syndrome (CRS) and severe neurotoxicity were not observed. Additionally, it was observed that 60% of the full cohort subjects did not develop any grade of CRS or neurotoxicity. No differences in incidence of CRS, neurotoxicity (NT), sCRS, or severe neurotoxicity (sNT) were observed between dose levels. Table 7 summarizes the incidence of cytokine release syndrome (CRS) and neurotoxicity adverse events in patients 28 days after receiving at least one dose of CAR-T cells. As shown in Table 7, no sCRS (Grade 3-4) was observed in any subjects that received a single dose of DL2 or double dose of DL1. Severe neurotoxicity or severe CRS (grade 3-4) was observed in 16% (9/55) of the full cohort of subjects and in 18% (8/44) of the subjects in the core subset. 11% (n=6) of subjects received tocilizumab, 24% (n=13) of subjects received dexamethasone. Among the ECOG2 subjects within the full cohort, observed rates of CRS and neurotoxicity were 71% and 29%, respectively.

TABLE 7
Assessment of Presence or Absence of CRS and Neurotoxicity
Adverse Events for Example 3.B.1

| | FULL | | | | | | | | | |
|---|---|---|---|---|---|---|---|---|---|---|
| | All Dose | | | | | | | | | |
| | Levels | | DL1S | | DL2S | | DL1D^{†} | | CORE | |
| Safety, N | 55 | | 30 | | 19 | | 6 | | 44 | |
| sCRS or | 9 | (16) | 6 | (20) | 2 | (11) | 1 | (17) | 8 | (18) |
| sNT, n (%) | | | | | | | | | | |
| CRS or NT, | 22 | (40) | 12 | (40) | 7 | (37) | 3 | (50) | 15 | (34) |
| n (%) | | | | | | | | | | |
| CRS | | | | | | | | | | |
| Grade 1-2, | 18 | (33) | 10 | (33) | 5 | (26) | 3 | (50) | 12 | (27) |
| n (%) | | | | | | | | | | |
| Grade 3-4, | 1 | (2) | 1 | (3) | 0 | | 0 | | 1 | (2) |
| n (%) | | | | | | | | | | |
| Neuro- | | | | | | | | | | |
| toxicity | | | | | | | | | | |
| Grade 1-2, | 3 | (6) | 1 | (3) | 2 | (11) | 0 | | 2 | (5) |
| n (%) | | | | | | | | | | |
| Grade 3-4, | 9 | (16) | 6 | (20) | 2 | (11) | 1 | (17) | 8 | (18) |
| n (%) | | | | | | | | | | |

^{†}Includes one patient treated at DL2 2-dose schedule due to dosing error

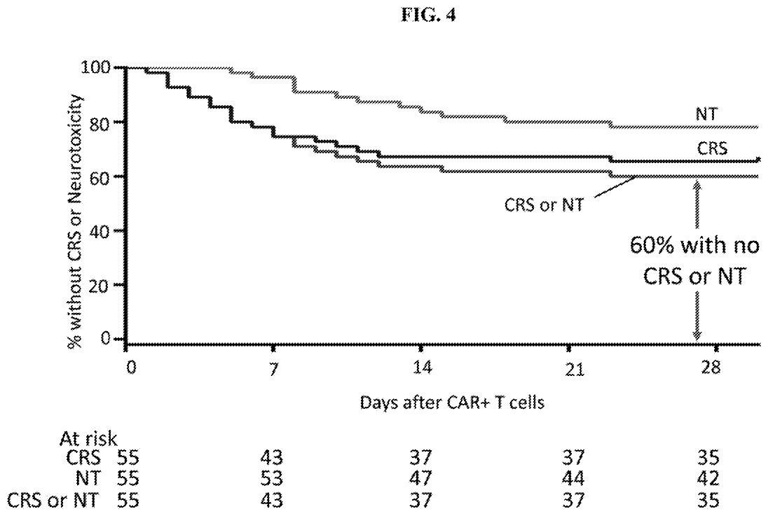

FIG. 4 shows a Kaplan meier curve depicting observed time to onset of CRS and/or neurotoxicity for the analysis in 3.B.1. As shown, the observed median times to onset of CRS and to onset of neurotoxicity were 5 and 11 days, respectively, with only 11% of patients experiencing onset of CRS less than 72 hours after initiation of the administration of the cell therapy. The median time to resolution of CRS and neurotoxicity to Grade 1 or better was 5 and 7 days, respectively. The median time to complete resolution of CRS and neurotoxicity was 5 and 11 days, respectively. The results were consistent with a conclusion that there was a low rate of early onset of any CRS or neurotoxicity in the subjects.

### Example 3.B.2

Example 3.B.2 describes assessment at the time-point in Example 3.B.2. Up to this time point, adverse event (AE) data were collected from lymphodepletion (LD) to 90 days post administration of CAR-expressing T cells. At the second time point, 69 subjects in the DLBCL cohort (full cohort) were evaluated for safety, 38 that had received DL1 single dose, 25 that had received DL2 single dose, and 6 having received DL1 double dose schedule. The most common TEAEs other than CRS or NT included neutropenia (41%, 28/69), fatigue (30%, 21/69), thrombocytopenia (30%, 21/69), and anemia (26%, 18/69). One Grade 5 TEAE of diffuse alveolar damage was observed.

No acute infusional toxicity was observed in the full cohort, and the majority of subjects, 64% (44/69), were observed to have no CRS or NT, indicating that outpatient delivery of CAR-expressing T cells may be possible. Rates of CAR T cell-associated toxicities, including CRS and NT, did not differ between dose levels. Safety profile was observed to be similar across cohorts and dose levels. Among the 25 subjects in the full cohort (36%) who experienced any grade CRS or NT, 21 (30%) had CRS and 14 (20%) had NT. No subjects had Grade 3 CRS and only one (1%, 1/69) had Grade 4 CRS and required ICU care; the other 29% (20/69) had Grade 1-2 CRS. Of the 20% of subjects with NT, 6% (4/69) had Grade 1-2 and 14% (10/69) had Grade 3-4; 2 (3%) had seizure. No Grade 5 CRS or grade 5 NT was observed. No incidences of cerebral edema were observed. All CRS and NT events were resolved except one case of Grade 1 tremor, which was ongoing at the time of analysis. Median time to onset of first CRS and NT was 5 days (range 2, 12) and 10 days (range 5, 23), respectively. In the first 72 hours post infusion, no subjects were observed to have NT, and only 10% (7/69) were observed to have CRS (all Grade 1); NT was preceded by CRS in >70% of subjects. Overall, thirteen (13) subjects (19%) required intervention for CRS or NT with anti-cytokine therapy (tocilizumab alone 1 (1%), dexamethasone alone 6 (9%), or both 6 (9%)) and only one required any vasopressor support. Median doses of tocilizumab and dexamethasone were 1 and 6, respectively. Median CRS and NT duration was 5 days and 11 days, respectively. Analysis of the core cohort (n=49) also showed similar rates of CRS and NT.

In this assessment, low incidences and late onsets of CRS and/or NT were observed, at both dose levels, supported the feasibility of outpatient infusion, such as with hospital admission at the first sign of fever or fever lasting beyond a certain period of time. No Grade 5 CRS or grade 5 NT was observed, and all severe CRS and severe NT were resolved. Further, approximately 2 out of 3 patients had no CRS or NT, supporting that the cells can be administered on outpatient basis. At the time of assessment in 3.B.2, four subjects had been treated in the outpatient setting. Further, no meaningful differences in toxicity was observed in subjects receiving DL1 or DL2, indicating achievement of higher response rates without an increase risk of toxicity or safety concerns.

C. Response Outcomes Following Treatment

Subjects were monitored for response, including by assessing tumor burden at 1, 3, 6, 7, 12, 18, and 24 months after administration of the CAR+ T cells.

### Example 3.C.1

Example 3.C.1 describes results based on the analysis time-point in Example 3.A.1 and 3.B.1.

Response rates are listed in Table 8. High durable response rates were observed in the cohort of subjects, which included subjects heavily pretreated or, with poor prognosis and/or with relapsed or refractory disease. For subjects across all doses in the Core (n=44) cohort, the observed overall response rate (ORR) was 86% and the observed complete response (CR) rate was 59%. At three months for the core cohort, the overall response rate (ORR) was 66%; the three-month CR rate was 50% among the core cohort. In the core cohort, the 3 month ORR was 58% (11/19) at dose level 1 and 78% at dose level 2; the 3 month CR rate was 42% (8/19) for dose level 1 and 56% (5/9) for dose level 2, consistent with a suggested dose response effect on treatment outcome. Additionally, the results were consistent with a relationship between dose and durability of response.

TABLE 8
Response

| | FULL | | | | CORE | | | |
|---|---|---|---|---|---|---|---|---|
| | All Dose | | | | All Dose | | | |
| | Levels | DL1S | DL2S | DL1D^{c} | Levels | DL1S | DL2S | DL1D^{a} |
| Best Overall | 54 | 30 | 18 | 6 | 44 | 25 | 15 | 4 |
| Response, | | | | | | | | |
| N^{a} | | | | | | | | |
| ORR, % | 76 | 80 | 72 | 67 | 86 | 84 | 87 | 100 |
| (95% CI) | (62, 87) | (61, 92) | (47, 90) | (23, 96) | (73, 95) | (64, 95) | (60, 98) | (40, 100) |
| CR, % | 52 | 53 | 50 | 50 | 59 | 56 | 60 | 75 |
| (95% CI) | (38, 66) | (34, 72) | (26, 74) | (12, 88) | (43, 74) | (35, 76) | (32, 84) | (19, 99) |
| ≥3 mos f/u, | 41 | 24 | 11 | 6 | 32 | 19 | 9 | 4 |
| n^{b} | | | | | | | | |
| 3 mo | 51 | 46 | 64 | 50 | 66 | 58 | 78 | 75 |
| ORR, % | (35, 67) | (26, 67) | (31, 89) | (12, 88) | (47, 81) | (34, 80) | (40, 97) | (19, 99) |
| (95% CI) | | | | | | | | |
| 3 mo CR, % | 39 | 33 | 46 | 50 | 50 | 42 | 56 | 75 |
| (95% CI) | (24, 56) | (16, 55) | (17, 77) | (12, 88) | (32, 68) | (20, 67) | (21, 86) | (19, 99) |

DL1S: DL1 1-dose schedule;
DL2S: DL2 1-dose schedule;
DL1D: DL1 2-dose schedule;
^{a}Included patients with event of PD, death, or 28 day restaging scans. Treated patients < 28 days prior to data snapshot were not included.
^{b}The denominator is number of patients who received the CAR T-cell therapy ≥ 3 months and a snapshot date with an efficacy assessment at Month 3 or prior assessment of PD or death.
^{c}Includes one patient treated at DL2 2-dose schedule due to dosing error

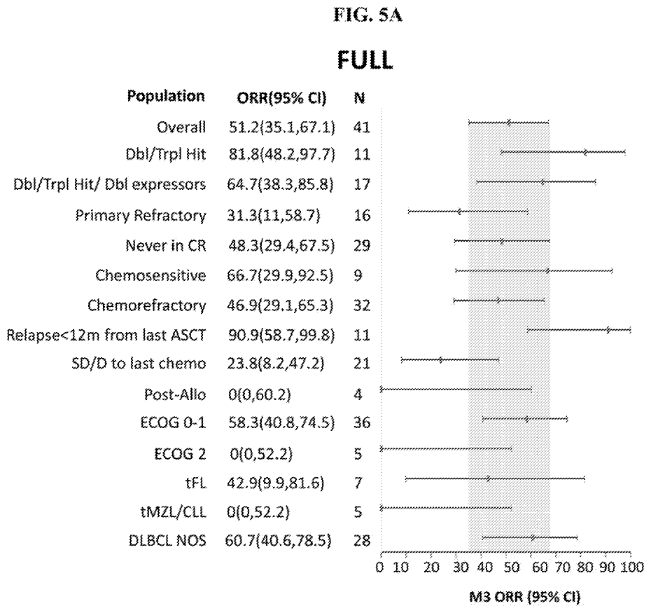

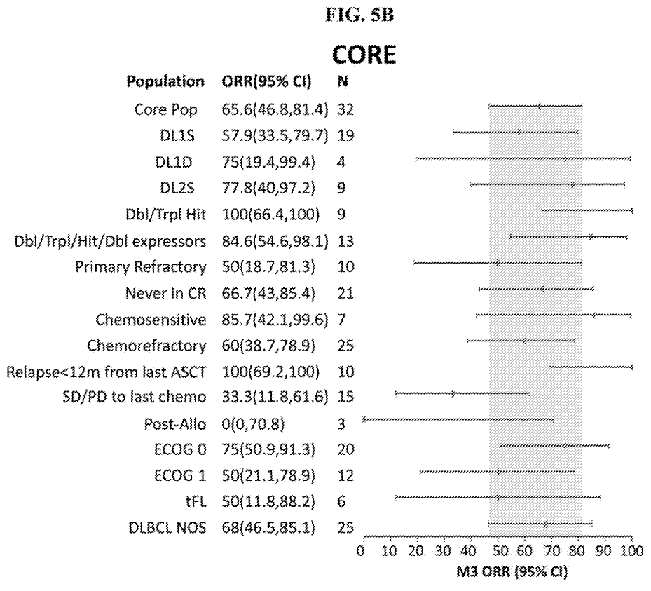

Overall response rates among various subgroups of subjects in the full and core cohorts are shown in FIG. 5A and 5B, respectively. In poor-risk DLBCL subgroups, response rates were generally high. An ORR of greater than 50% was observed at 3 months in patients with double/triple hit molecular subtype, that had primary refractory or chemorefractory DLBCL or that never before had achieved a CR. Complete resolution of CNS involvement by lymphoma was observed in 2 patients.

Among the subjects treated six months or greater prior to the particular time-point of the evaluation, of the ten (10) patients that had been in response at three months, 9 (90%) remained in response at six months. At the evaluation time-point, 97% of subjects in the core subset who had responded were alive and in follow-up, median follow-up time 3.2 months.

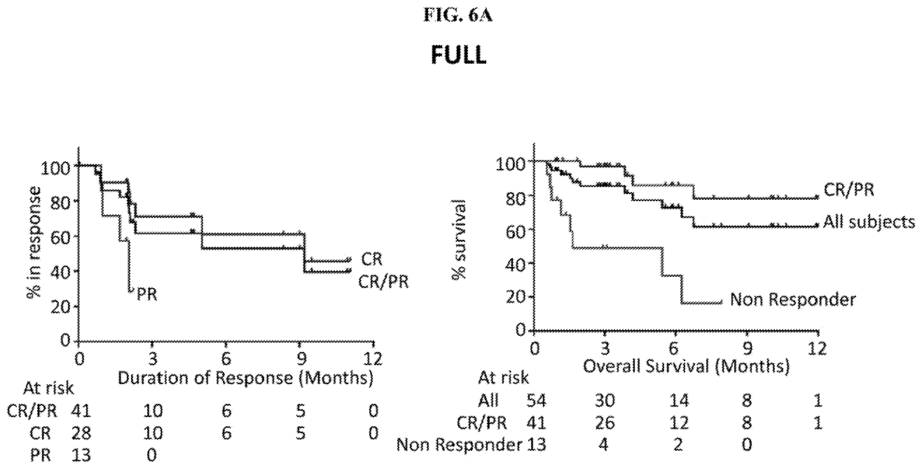

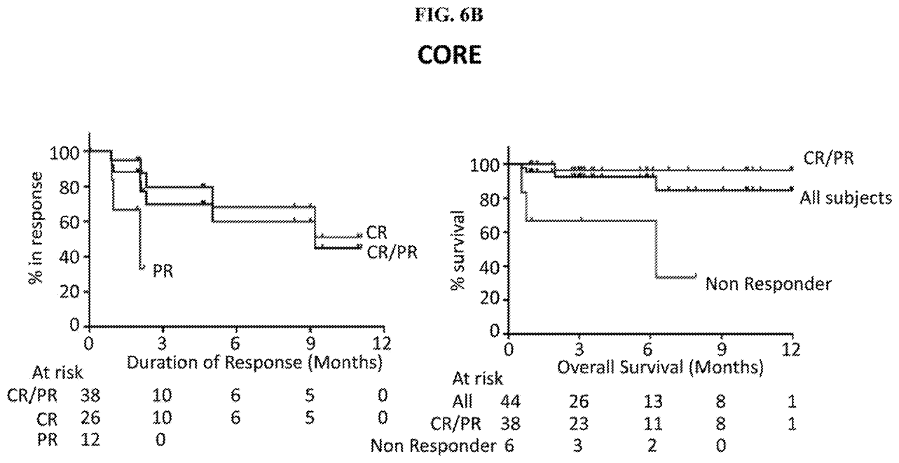

Results for the duration of response and overall survival (grouped by best overall response (non-responder, CR/PR, CR and/or PR)) are shown for full and core cohorts of subjects, in FIGS. 6A and 6B, respectively. As shown, prolonged survival was observed in responders, with increased durability of response in subjects with CRs. All patients in response at three months remained alive at the time of evaluation, although 5/6 subjects with poor performance status (ECOG 2) had expired.

### Example 3.C.2

Example 3.C.2 describes results based on the analysis time-point in Example 3.A.2 and 3.B.2.

Up to the time point in Example 3.C.2, 68 subjects in the full DLBCL cohort was evaluated for response. Overall or objective response (OR), 3-month, and 6-month objective response rates were 75% (51/68), 49% (27/55), and 40% (14/35), respectively. Complete response (CR) rate, 3-month CR rate, and 6-month CR rate were 56% (38/68), 40% (22/55), and 37% (13/35), respectively. A trend toward improved response rate at 3 months was observed in subjects treated at DL2 compared to DL1: 63% (12/19; 95% CI 38, 84) vs 40% (12/30; 95% CI 23, 59) for ORR with p=0.148, and 58% (11/19; 95% CI 34, 80) vs 27% (8/30; 95% CI: 12, 46) for CR with p=0.0385. Among 16 double/triple hit lymphoma subjects, ORR was 81%, and 3-month CR rate was 60%.

In the core cohort (n=49 for the time-point in Example 1.C.2), OR, 3-month, and 6-month OR rates were 84% (41/49), 65% (26/40), and 57% (13/23), respectively. CR rate, 3-month CR rate, and 6-month CR rate were 61% (30/49), 53% (21/40), and 52% (12/23), respectively. A similar trend in improved durable ORR and CR at 3 months at higher doses was observed. Specifically, for patients in the CORE cohort administered DL2, 3-month ORR was 80% (12/15; 95% CI 52, 96) and 3-month CR was 73% (11/15; 95% CI 45, 92), compared to 3-month ORR and CR rates of 52% (11/21; 95% CI 30, 74) and 33% (7/21; 95% CI 15, 57) in CORE cohort subjects administered DL1, with p=0.159 and p=0.0409 respectively. Among subjects in the CORE cohort having received DL2 and with 3-month follow-up (n=15), 3-month ORR was 80% and 3-month CR was 73%.

Median DOR in the full cohort and core cohorts at this time-point in 1.C.2 was 5.0 and 9.2 months, respectively; median duration of CR was 9.2 months in the full cohort. Median duration of CR had not been reached in the core cohort. Median overall survival (OS) was 13.7 months in the full cohort and had not been reached in the core cohort. 6-month OS was 75% in the full cohort, with median follow-up of 5.8 months. 6-month OS was 88% in the core cohort, with median follow up of 5.6 months.

D. Assessment of CAR⁺ T Cells in Blood

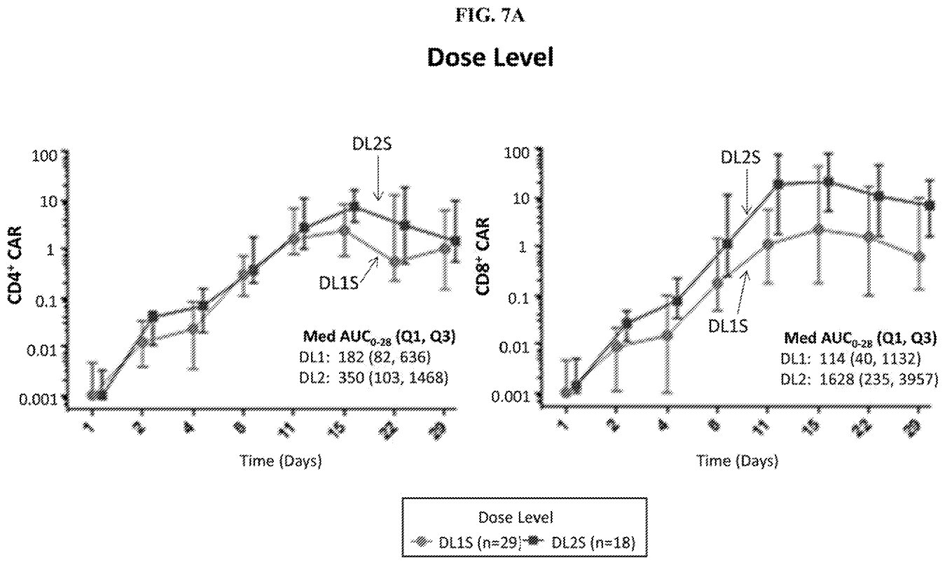

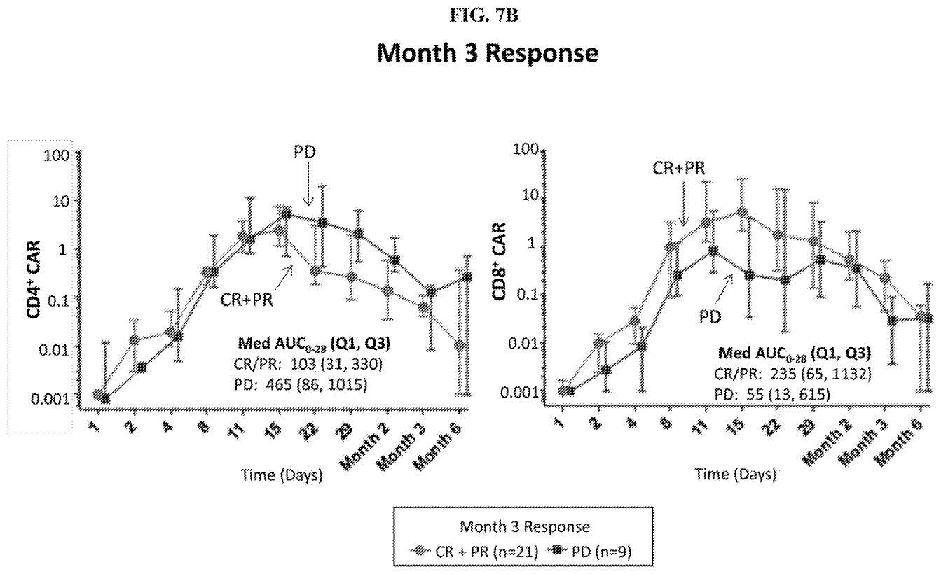

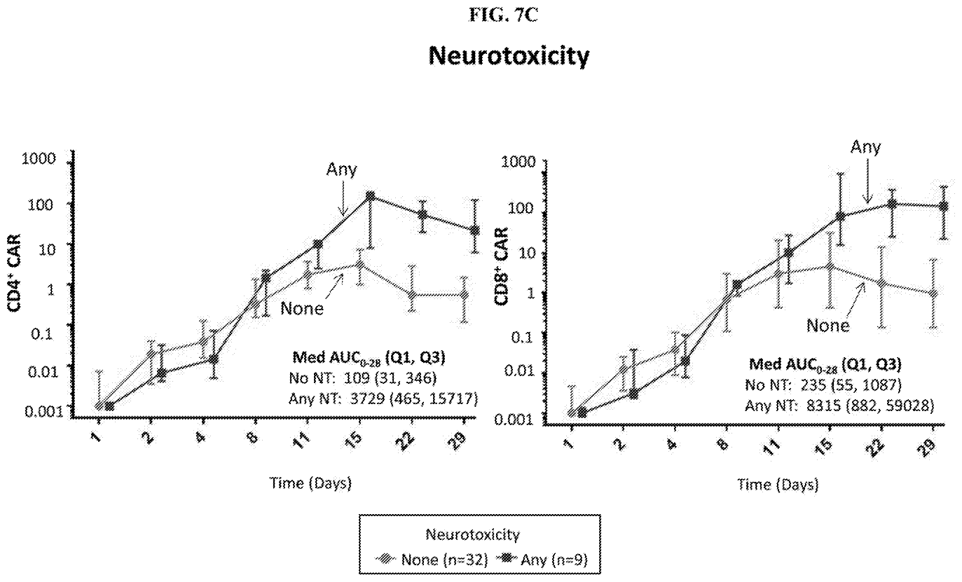

Based on data from the time-point described in Example 3.A.1, 3.B.1 and 3.C.1, pharmacokinetic analysis was carried out to assess numbers of CAR⁺ T cells in peripheral blood at various time points post-treatment. Results from the fifty-five (55) subjects assessed at the time-point in Example 3.A.1 in the DLBCL cohort and four (4) subjects (assessed at that same time-point) in the mantle cell lymphoma (MCL) described in Example 4 below were analyzed. Pharmacokinetics (PK) measurements were carried out using validated flow cytometry to detect a marker expressed in the CAR construct and quantitative PCR-based assays to detect the integration of the CAR construct. B cell aplasia was assessed by flow cytometry using anti-CD19 antibodies. As shown in FIG. 7A, CD4⁺ and CD8⁺CAR-expressing cells, as measured by the number of cells/μL blood (median±quartiles) plotted on a log scale, were detected throughout the course of assessment at both administered dose levels. Subjects receiving DL2 relative to DL1 had higher median Cₘₐₓ and median AUC₀₋₂₈ for CD3⁺/CAR⁺, CD4⁺/CAR⁺, and CD8⁺/CAR⁺ T cell subsets in peripheral blood (AUC₀₋₂₈: DL2 vs. DL1 was 1836 vs. 461, 350 vs. 182, and 1628 vs. 114, for CD3⁺, CD4⁺, and CD8⁺, respectively; p<0.05 for CD8⁺; Cₘₐₓ: DL2 vs. DL1 was 99.8 vs. 27.9, 15.1 vs. 5.2, and 73.1 vs. 5.5 cells/μL, respectively). Median time to maximum CD3⁺CAR⁺ T cell expansion was 15 days (range 8-29) and did not differ between dose levels. CD4⁺ and CD8⁺CAR-expressing T cells homed to the bone marrow at relatively similar levels.

An increased median area under the curve (AUC) (CD8⁺CAR⁺ T cell numbers over time in the blood) was observed among subjects administered the higher dose level, as compared to the lower dose level, without an observed increase in toxicity. Higher peak CD8⁺/CAR⁺ T cell exposure was observed in responders (CR/PR) than non-responders (PD); persistence of cells over the time of assessment, including out to 3 and 6 months, was observed even in subjects whose disease had progressed (FIG. 7B). Median Cₘₐₓ and median AUC₀₋₂₈ of CD8⁺CAR⁺ T cells were higher in responding subjects and with durable response at month 3 (CD8⁺Cₘₐₓ median=20.8 vs. 5.5; CD8⁺ AUC₀₋₂₈median=235 vs. 55 in CR/PR at Month 3 vs. PD at Month 3). Among subjects that were evaluated for CAR T cell persistence, 90% and 93% of 29 subjects had detectable CD8⁺ and CD4⁺CAR⁺ T cells, respectively, at month 3; 63% and 58% of 19 subjects had detectable CD8⁺ and CD4⁺ CAR⁺ T cells, respectively, at month 6. At months 3 and 6, no statistically significant differences in the persistence of CAR⁺ T cells were observed between subjects with durable response or relapse. CAR⁺ T cells were detectable at time of relapse in 89% of 11 subjects with PK, even though B cell aplasia (<1 cell/μl) was demonstrated in nearly all subjects 97% (34/35) at month 3, and 100% (24/24) at month 6.

Higher Cₘₐₓ and AUC₀₋₂₈ at DL2 compared to DL1 was not observed to be associated with increased CRS or NT. For any NT or for >Grade 2 CRS, median AUCs of CD4⁺/CAR⁺ and CD8⁺/CAR⁺ T cells were 5 to 10 fold and 3 to 5 fold higher, respectively, than the median AUC for DL2. Higher disease burden and baseline levels of inflammatory cytokines was observed to be associated with higher peak levels of CAR⁺ T cells, higher cytokine peak levels, and higher incidences of CRS and NT. The results were consistent with a conclusion that the higher Cmax and median AUCO-28 at DL2 did not increase CRS or NT.

The results were consistent with a conclusion that treatment resulted in prolonged exposure and persistence of the engineered cells, even in subjects with poor responses. In some embodiments, combination approaches are used, such as administration of an immune checkpoint modulator or other immune modulatory agent, e.g., following relapse or disease progression, at a time at which engineered cells persist in the subject, e.g., as measured by levels of cells in peripheral blood. In some aspects, the cells, having persisted for a prolonged period, re-expand or become activated and/or exhibit anti-tumor function, following administration of the other agent or treatment. Higher median CD4+ and CD8+ CAR+ T cell numbers were generally observed over time in blood of subjects who developed neurotoxicity (FIG. 7C). Results indicated that the CAR⁺ T cells exhibited expansion and persistence, durability of response at 3 months that increased at higher dose levels, without increased toxicity. Results were observed that were consistent with a suggestion that high peak levels of CAR⁺ T cells and cytokines in the blood may be associated with NT and CRS, and may be influenced by baseline subject factors. It was observed that CAR⁺ T cells were present at the time of relapse, indicating that combination or retreatment approaches may provide certain advantages.

E. Blood Analytes and Neurotoxicity, CRS and Response

Various pre-treatment blood analytes, including cytokines, were measured in the serum of subjects (those assessed at the time-point in Example 3.A.1),prior to administration of the CAR+ T cells. Cytokines were measured using a multiplex cytokine assay. Potential correlations to risk of developing neurotoxicity were assessed using statistical analysis based on univariate nonparametric tests.

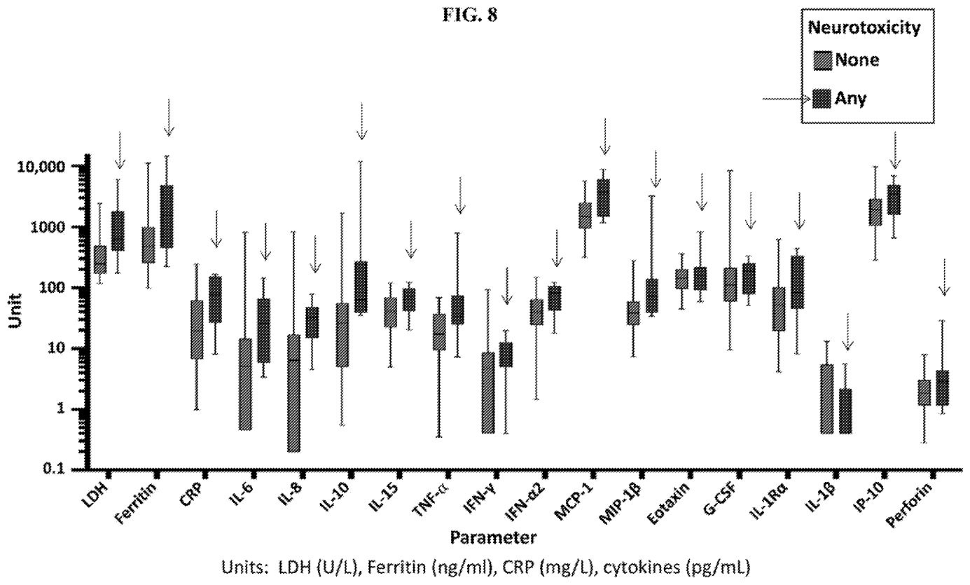

FIG. 8 shows median levels of the assessed analytes in units (LDH, U/L; ferritin, ng/mL; CRP, mg/L; cytokines, pg/mL) in subjects that did not develop a neurotoxicity versus subjects that did develop a neurotoxcity following CAR+ T cell therapy. Levels of certain blood analytes, including LDH, Ferritin, CRP, IL-6, IL-8, IL-10, TNF-α, IFN-α2, MCP-1 and MIP-1β, were observed to be associated with level of risk of developing neurotoxicity (Wilcoxon p values <0.05, without multiplicity adjustment). In particular, the results were consistent with a conclusion that pre-treatment levels of LDH, which in some embodiments is a surrogate for disease burden, may be useful for potential neurotoxicity risk assessment and/or risk-adapted dosing or adjustment of treatment of certain subjects. In addition, tumor burden measured before administration of the CAR-T cell composition correlated (Spearman p values <0.05) with the risk of developing neurotoxicity. In some aspects, LDH levels may be assessed alone and/or in combination with another pre-treatment parameter, such as another measure or indicator of disease burden, such as a volumetric tumor measurement such as sum of product dimensions (SPD) or other CT-based or MRI-based volumetric measurement of disease burden. In some aspects, one or more parameters indicative of disease burden are assessed, and in some contexts may indicate the presence, absence or degree of risk of developing neurotoxicity following the T cell therapy. In some aspects, the one or more parameters include LDH and/or a volumetric tumor measurement.

In an additional analysis, fifty-five (55) subjects in the DLBCL cohort at the time-point in Example 3.A.1, and four (4) subjects in the mantle cell lymphoma (MCL) described in Example 4 below were included in analysis for correlation with safety evaluations. In the 59 subjects evaluated for safety, CRS was observed in 32% (30% Grade 1-2, 0% Grade 3, 2% Grade 4); NT was observed in 20% (5% Grade 1-2, 10% Grade 3, 5% Grade 4). Dose level did not correlate with CRS or NT (p=0.565 and p=1.00, respectively). Subject factors that correlate with any grade CRS and NT were poorer performance status (e.g. ECOG Status 2) (p=0.03) and higher disease burden (p<0.05) as measured by the sum of the products of diameters (SPD) based on imaging results. Pre-CAR+ T cell infusion clinical laboratory parameters and cytokine measurements for pre-CAR+ T cell infusion that were observed to be associated with the occurrence of any grade NT included higher serum LDH, ferritin, and CRP, and higher plasma IL-6, IL-8, IL-10, TNF-α, IFN-α2, MCP-1, and MIP-1β (p<0.05 for each). Higher pre-CAR+ T cell infusion plasma levels of IL-8, IL-10, and CXCL10 were also associated with Grade 3-4 NT (p<0.05 for each).

Of the 54 subjects in the DLBCL cohort that were evaluated for response, higher ECOG scores and DLBCL transformed from CLL or MZL correlated with lower durable response at month 3 (p=0.02 for both). Pre-CAR+ T cell infusion parameters associated with best ORR included lower values of ferritin, LDH, CXCL10, G-CSF, and IL-10, and those associated with durable response at 3 months included lower ferritin, CRP, LDH, CXCL10, IL-8, IL-10, IL-15, MCP-1, MIP-1β, TNF-α, and higher pre-CAR+ T infusion hemoglobin and albumin (p<0.05 for each).

In some cases, the apheresis sample and CAR+ T cell composition for administration was assessed and correlated with clinical outcomes. The results showed that T cell memory subsets and T cell functionality may correlate with certain clinical outcomes.

The results showed that certain baseline patient characteristics, including inflammatory state and high tumor burden prior to treatment, may be useful for the identification of patients at risk for increased toxicity following administration of CAR-expressing T cells. Low tumor burden and low inflammatory state were observed to be associated with improved toxicity profile and better durability of response. The results support that treating subjects earlier in the course of therapy and/or assessing a panel of clinical and laboratory biomarkers to risk stratify subjects for potential early intervention may mitigate the risk of toxicity and improve durability of response.

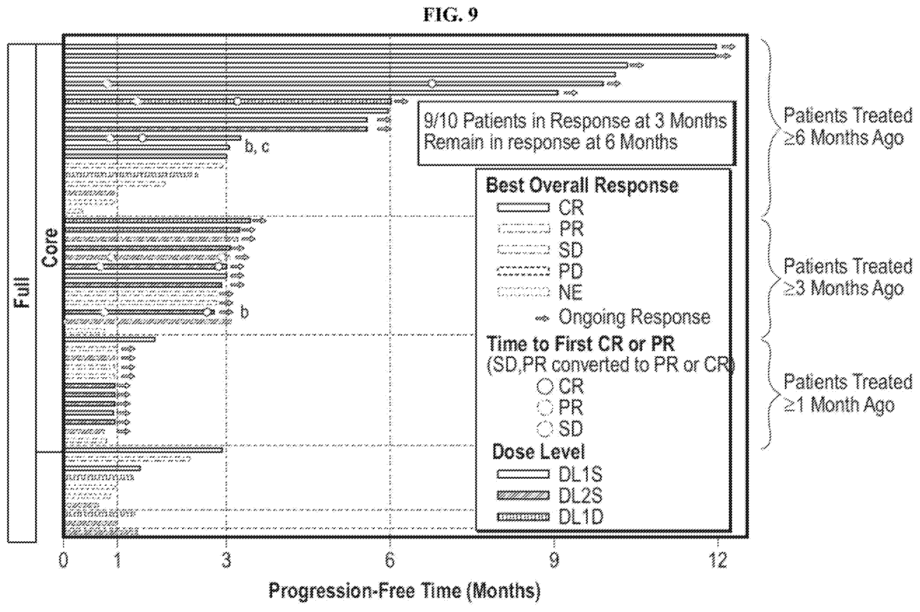

FIG. 9 shows a graph plotting progression-free time (months) for individual subjects within the full and core cohorts. Each bar represents a single patient. Shading indicates best overall response (in each case, unless otherwise indicated, achieved at 1 month); texture indicates dose (solid=dose level 1, single dose; cross-hatched, dose-level 2, single dose; vertical hatched=dose level 1, two-dose). Horizontal arrows indicate an ongoing response. Certain individual subjects were initially assessed (e.g., at 1-month) as exhibiting stable disease (SD) or Partial Response (PR), and were later observed to have achieved a PR (e.g., conversion of SD to PR) or CR. In such cases, shading of the individual patient bar, as noted, indicates best overall response, and dots (same correspondence of shading to response achieved) along each individual subject bar, indicate when each SD, PR, and/or CR was observed to have occurred in the subject. Complete resolution of CNS involvement by lymphoma was observed in two patients. CAR+ cells in one subject were observed to have expanded following biopsy after relapse.

### Example 4

### Administration of Anti-CD19 CAR-Expressing Cells to Subjects with Mantle Cell Lymphoma (MCL)

Therapeutic CAR+ T cell compositions containing autologous T cells expressing a chimeric antigen-receptor (CAR) specific for CD19, generated as described in Example 1, were administered to four (4) human subjects with mantle cell lymphoma (MCL) that had failed 1 line of therapy. The cryopreserved cell compositions were thawed prior to intravenous administration. The therapeutic T cell composition was administered as a defined composition cell product with formulated CD4+ and CD8+ populations of CAR+ engineered T cells derived from the same subject administered at a target ratio of approximately 1:1. Subjects were administered a dose of CAR-expressing T cells (as a split dose of the CD4+ and CD8+CAR-expressing T cells) at a single dose of dose level 1 (DL1) containing 5×10⁷ CAR-expressing T cells. Beginning at three (3) days prior to CAR+ T cell infusion, subjects received a lymphodepleting chemotherapy with fludarabine (flu, 30 mg/m²) and cyclophosphamide (Cy, 300 mg/m²).

Subjects were monitored for response and toxicities as described in Example 1. No CRS or neurotoxicity was observed in any of the subjects. Of the 4 subjects that were treated, two (2) subjects achieved PR (not durable) and two (2) patients had progressive disease.

### Example 5

### Biomarker Assessment in Pre- and Post-Administration Tumor Biopsies from Subjects with Relapsed and Refractory Non-Hodgkin's Lymphoma (NHL) for Administration of Anti-CD19 CAR-Expressing Cells

Expression of several biomarkers was assessed in tumor biopsies collected from subjects before and/or after administration of CAR-expressing cells.

### A. Tumor Biopsy Samples

Tumor biopsies were collected from selected subjects with relapsed or refractory (R/R) diffuse large B-cell lymphoma (DLBCL) or mantle cell lymphoma (MCL) who received treatment with therapeutic CAR⁺ T cell compositions containing autologous T cells expressing a chimeric antigen-receptor (CAR) specific for CD19, described above in Examples 3 and 4 above, based on the time point of assessment in Example 3.A.2. Tumor biopsies were obtained prior to administration of the CAR⁺ T cells (pre-treatment) and at 7 to 20 days after administration (post-treatment). Results are described in this example for evaluation through the time-point in Example 3.A.2, in an ongoing study. Results from 43 biopsies (26 pre-treatment; 17 post-treatment and 15 matched pairs) from 28 total subjects (25 DLBCL and 3 MCL) were examined.

### B. Assessment of Biomarkers, Response and Safety Outcomes

Infiltration of CAR⁺ T cell in the tumor biopsy was quantified using in situ hybridization (ISH) probes specific to the mRNA encoding the anti-CD19 CAR. CAR⁺ T cells, non-CAR T cells and B cells were enumerated using multiplex immunofluorescence (IF) assays detecting for a cell surface surrogate marker for CAR-expressing cells, CD4, CD8, CD19, CD20, CD73, FOXP3, CD163, IDO and PD-L1. Tumor biopsy sections were stained with hematoxylin and eosin (H&E) and assessed for tissue quality and tumor identification. Immunofluorescence images were analyzed using an image analysis software. Potential correlations to response outcomes were assessed using statistical analysis based on univariate t-tests, and the p-values were 2-sided without multiplicity adjustment.

Subjects were assessed for response and safety outcomes, including by assessing the tumor burden at various time points after administration of the CAR⁺ T cells, including at 3 months after administration, and determining whether the subject had progressive disease (PD), stable disease (SD), partial response (PR), or complete response (CR). Safety outcomes evaluated included neurotoxicity (neurological complications including symptoms of confusion, aphasia, encephalophathy, myoclonus seizures, convulsions, lethargy, and/or altered mental status), graded on a 1-5 scale, according to the National Cancer Institute—Common Toxicity Criteria (CTCAE) scale, version 4.03 (NCI-CTCAE v4.03).

### C. Results

The observed objective response rate (ORR; including CR and PR) was 71% (20/28) in the subjects for which biopsies were assessed. Grade 1, 2 CRS was observed in 36% (10/28; grade 1, 2) of the subjects for which biopsies were assessed, and Grades 2-4 NT was observed in 18% (5/28) of the subjects for which biopsies were assessed.

Pre-treatment tumor biopsies were observed to contain varying cellular compositions: tumor cells (median: 77%; range 5-96%), CD4⁺ cells (0.90%; 0.02-15%), and CD8⁺ cells (1.5%; 0-23%). The results showed that subjects with a CR or PR at 3 months after CAR+ T cell administration had a higher percentage of endogenous CD4⁺ cells in pre-treatment tumors compared those with a PD (CR, PR median: 7.9%; PD median: 0.38%; p<0.0001). Percentages of CD8⁺ cells in pre-treatment tumors did not differ between the 3 month response groups (CR, PR median: 1.9%; PD median: 0.47%; p=0.6496).

In the post-treatment biopsies, CAR+ T cell were observed to have infiltrated the tumor, and constituted up to 22% of cells in the biopsy sample. The level of tumor infiltration in post-treatment samples (7 to 20 days after administration) was observed to be higher in subjects that went on to achieve a CR (median: 3.9%) or PR (median: 1.1%) compared to subjects that went on to achieve a best overall response (BOR) of SD or PD (median: 0.51%). Although both CD4⁺ and CD8⁺CAR T cells were observed to have infiltrated the tumor area at the post-treatment time point (7 to 20 days after administration), subjects that went on to achieve a CR were observed to have higher ratio of CD8⁺CAR⁺ T cells to CD4⁺CAR⁺ T cells, at this post-treatment timepoint, as compared to subjects that went on to achieve a BOR of SD or PD (CR median: 0.83; SD, PD median: 0.14; p=0.0097).

Comparing matched pre- and post-treatment biopsies from individual subjects, results showed a trend towards subjects ultimately achieving a BOR of CR or PR having a larger post-treatment increase in CD8⁺ cells (CAR⁺ T and non-CAR T) in tumors, as compared to subjects ultimately achieving a BOR of SD or PD (CR, PR median change: +5.3%; SD, PD median change: +0.06%; p=0.1225).

Expression of immunosuppressive factors, including CD73, FOXP3, CD163, IDO and PD-L1, varied among subjects at pre-treatment (CD73 (median: 1.5%; range 0-42%), FOXP3 (0.10%; 0-1.5%), IDO (0.06%; 0-11%), CD163 (1.2%; 0-24%) and PD-L1 (0.16%; 0-56%)) and post-treatment (CD73 (1.6%; 0-53%), FOXP3 (0.09%; 0-4.3%), IDO (0.28%; 0-15%), CD163 (3.6%; 0-22%) and PD-L1 (3.3%; 0-65%)). Post-treatment increases in CD8⁺ cells in matched biopsies were observed to be associated with post-treatment increases in IDO (R²=0.64) and PD-L1 (R²=0.61) expression. This result is consistent with a conclusion that infiltration of CD8+ CAR+ cells at the time assessed may indicate potential likelihood of achieven a degree of response or duration of response, and that the presence and/or activity of such cells may result in upregulation of TME factors.

### D. Conclusion

Durable response at month 3 after CAR+ T cell administration was observed to be associated with higher levels of CD4⁺ cells in pre-treatment tumors. In post-treatment tumor cells, CAR+ T cells, both CD4⁺ and CD8⁺, were observed to infiltrate the tumor and adjacent tissue. ORR was associated with an increase in CAR+ T cells in the tumor biopsy. An increase of CD8⁺levels in the post-treatment tumor biopsy compared to CD8⁺levels in the pre-treatment tumor biopsy was associated with increased IDO and PD-L1 expression. In some embodiments, therapies targeting these pathways, such as those administered at the time of or following administration of the CAR-T cells, may enhance one or more therapeutic outcomes or duration thereof following CAR+ T cell administration.

### Example 6

### Assessment of Persistence in Subjects with Relapsed and Refractory Non-Hodgkin's Lymphoma (NHL) After Administration of Anti-CD19 CAR-Expressing Cells

Persistence and expansion was assessed in patients at a subsequent point in time in the clinical study described in Example 3 above.

### A. Subjects, Response and Safety

The analysis at this time point presented in this example is based on assessment of a total of 91 subjects in the full DLBCL cohort (88 (34 from the CORE cohort) assessed for response and 91 assessed for safety) that had been administered the anti-CD19 CAR-expressing cells. As shown in Table 9. The objective response rate (ORR) was 74%, including 52% subjects who showed a complete response (CR). The incidence of any grade of cytokine release syndrome (CRS) was 35%, with 1% severe CRS; and the incidence of any grade of neurotoxicity (NT) was 19%, with 1% severe NT.

TABLE 9
Response and Safety After CAR+ Cell Administration

| | FULL | CORE | | |
|---|---|---|---|---|
| | All Dose | All Dose | | |
| | Levels | Levels^{a} | DL1S | DL2S |
| Best Overall Response | 88 | 65 | 34 | 27 |
| (BOR), n^{b} | | | | |
| ORR, % (95% CI) | 74 (63, 83) | 80 (68, 89) | 77 (59, 89) | 82 (62, 94) |
| CR, % (95% CI) | 52 (41, 63) | 55 (43, 68) | 47 (30, 65) | 63 (42, 81) |
| Safety, n^{c} | 91 | 67 | 34 | 29 |
| Any CRS, % (95% CI) | 35 (25, 46) | 36 (24, 48) | 41 (25, 59) | 24 (10, 44) |
| sCRS(grade 3-4), % (95% CI) | 1 (0, 6) | 1 (0, 8) | 38 (0, 15) | 0 |
| Any NTx, % (95% CI) | 19 (11, 28) | 21 (12, 33) | 24 (11, 41) | 17 (6, 36) |
| sNTx(grade 3-4), % (95% CI) | 12 (6, 21) | 15 (7, 26) | 21 (9, 38) | 7 (1, 23) |

^{a}Four patients treated on DL1D (dose level 1, two-dose schedule) with similar outcomes.
^{b}Includes patients with event of PD, death, or 28-day restaging scans. One patient did not have restaging scans available.
^{c}Includes all subjects who have received at least one dose of conforming CAR-expressing cell product 28 days prior to data snapshot date or died.

### B. Persistence

Persistence of CAR-expressing cells and CD19+ B cell aplasia (low numbers or absence of CD19+ B cells) was assessed at various time points in evaluable subjects with DLBCL that had been administered CAR+ T cells, based on detectable CD3⁺, CD4⁺ or CD8⁺ CAR-expressing cell levels and levels of CD19⁺ B-cells detected in the blood, respectively. The results are set forth in Table 10. Among subjects evaluated at progression (n=37), a median of 0.17 CD4+ CAR+ cells/μL (range, 0-65.5 cells/μL)-expressing cell at progression was and a median of CD8+ CAR+ 0.15 cells/μL (range, 0-131.8 cells/μL) were observed at progression. Among subjects evaluated at relapse (progression after achieving CR/PR) (n=12), a median of 0.17/μL (range, 0-35.1 cells/μL) CD4+ CAR-expressing cells and a median of 0.20 cells/μL (range, 0-131.8 cells/pt). CD8+ CAR-expressing cells were observed at relapse Long-term persistence of CAR-expressing cells was observed in 75% of evaluable subjects with DLBCL at 12 months. Long-term persistence of B cell aplasia also was observed in 75% of the subjects at 12 months, and in subjects regardless of relapse status. The results are consistent with a conclusion that the anti-CD19 CAR-expressing cells exhibited long-term persistence in most subjects, and suggest the potential for ongoing, low-level disease control even in relapsed patients.

Of subjects who relapsed, 91.7% (11/12) had detectable CAR-expressing cells in the blood at the time of relapse. This result is consistent with a conclusion that a combination therapy or other intervention in some embodiments may be used to augment and/or boost CAR-expressing cells such as those that may be exhausted.

TABLE 10
CAR+ Cell Long-Term Persistence and CD19 Aplasia

| | | | | Month | At | At |
|---|---|---|---|---|---|---|
| | Month 3 | Month 6 | Month 9 | 12 | Progression | Relapse |
| CAR T persistence in | 50 | 30 | 18 | 12 | 37 | 12 |
| evaluable patients, n | | | | | | |
| CD3⁺, % | 100 | 80.0 | 77.8 | 75.0 | 91.9 | 91.7 |
| CD4⁺, % | 88.0 | 63.3 | 50.0 | 41.7 | 83.8 | 83.3 |
| CD8⁺, % | 90.0 | 70.0 | 55.6 | 50.0 | 83.8 | 75.0 |
| CD19⁺ B-cell aplasia | 96.0 | 93.3 | 77.8 | 75.0 | 97.3 | 100 |
| (<1 cell/μL), % | | | | | | |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID | | |
| NO. | SEQUENCE | DESCRIPTION |
| 1 | ESKYGPPCPPCP | spacer |
| | | (IgG4hinge)(aa) |
| | | Homo sapiens |
| 2 | GAATCTAAGTACGGACCGCCCTGCCCCCCTTGCCCT | spacer |
| | | (IgG4hinge)(nt) |
| | | Homo sapiens |
| 3 | ESKYGPPCPPCPGQPREPQVYTLPPSQEEMTKNQVSLTCLVKGFYPSDIAVEWE | Hinge-CH3 spacer |
| | SNGQPENNYKTTPPVLDSDGSFFLYSRLTVDKSRWQEGNVFSCSVMHEALHNHY | Homo sapiens |
| | TQKSLSLSLGK | |
| 4 | ESKYGPPCPPCPAPEFLGGPSVFLFPPKPKDTLMISRTPEVTCVVVDVSQEDPE | Hinge-CH2-CH3 |
| | VQFNWYVDGVEVHNAKTKPREEQFNSTYRVVSVLTVLHQDWLNGKEYKCKVSNK | spacer |
| | GLPSSIEKTISKAKGQPREPQVYTLPPSQEEMTKNQVSLTCLVKGFYPSDIAVE | Homo sapiens |
| | WESNGQPENNYKTTPPVLDSDGSFFLYSRLTVDKSRWQEGNVFSCSVMHEALHN | |
| | HYTQKSLSLSLGK | |
| 5 | RWPESPKAQASSVPTAQPQAEGSLAKATTAPATTRNTGRGGEEKKKEKEKEEQE | IgD-hinge-Fc |
| | ERETKTPECPSHTQPLGVYLLTPAVQDLWLRDKATFTCFVVGSDLKDAHLTWEV | Homo sapiens |
| | AGKVPTGGVEEGLLERHSNGSQSQHSRLTLPRSLWNAGTSVTCTLNHPSLPPQR | |
| | LMALREPAAQAPVKLSLNLLASSDPPEAASWLLCEVSGFSPPNILLMWLEDQRE | |
| | VNTSGFAPARPPPQPGSTTFWAWSVLRVPAPPSPQPATYTCVVSHEDSRTLLNA | |
| | SRSLEVSYVTDH | |
| 6 | LEGGGEGRGSLLTCGDVEENPGPR | T2A |
| | | artificial |
| 7 | MLLLVTSLLLCELPHPAFLLIPRKVCNGIGIGEFKDSLSINATNIKHFKNCTSI | tEGFR |
| | SGDLHILPVAFRGDSFTHTPPLDPQELDILKTVKEITGFLLIQAWPENRTDLHA | artificial |
| | FENLEIIRGRTKQHGQFSLAVVSLNITSLGLRSLKEISDGDVIISGNKNLCYAN | |
| | TINWKKLFGTSGQKTKIISNRGENSCKATGQVCHALCSPEGCWGPEPRDCVSCR | |
| | NVSRGRECVDKCNLLEGEPREFVENSECIQCHPECLPQAMNITCTGRGPDNCIQ | |
| | CAHYIDGPHCVKTCPAGVMGENNTLVWKYADAGHVCHLCHPNCTYGCTGPGLEG | |
| | CPTNGPKIPSIATGMVGALLLLLVVALGIGLFM | |
| 8 | FWVLVVVGGVLACYSLLVTVAFIIFWV | CD28 (amino |
| | | acids 153-179 of |
| | | Accession No. |
| | | P10747) |
| | | Homo sapiens |
| 9 | IEVMYPPPYLDNEKSNGTIIHVKGKHLCPSPLFPGPSKP | CD28 (amino |
| | FWVLVVVGGVLACYSLLVTVAFIIFWV | acids 114-179 of |
| | | Accession No. |
| | | P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | P10747) |
| | | Homo sapiens |
| 11 | RSKRSRGGHSDYMNMTPRRPGPTRKHYQPYAPPRDFAAYRS | CD28 (LL to GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) |
| | | Homo sapiens |
| 13 | RVKFSRSADAPAYQQGQNQLYNELNLGRREEYDVLDKRRGRDPEMGGKPRRKNP | CD3 zeta |
| | QEGLYNELQKDKMAEAYSEIGMKGERRRGKGHDGLYQGLSTATKDTYDALHMQA | Homo sapiens |
| | LPPR | |
| 14 | RVKFSRSAEPPAYQQGQNQLYNELNLGRREEYDVLDKRRGRDPEMGGKPRRKNP | CD3 zeta |
| | QEGLYNELQKDKMAEAYSEIGMKGERRRGKGHDGLYQGLSTATKDTYDALHMQA | Homo sapiens |
| | LPPR | |
| 15 | RVKFSRSADAPAYKQGQNQLYNELNLGRREEYDVLDKRRGRDPEMGGKPRRKNP | CD3 zeta |
| | QEGLYNELQKDKMAEAYSEIGMKGERRRGKGHDGLYQGLSTATKDTYDALHMQA | Homo sapiens |
| | LPPR | |
| 16 | RKVCNGIGIGEFKDSLSINATNIKHFKNCTSISGDLHILPVAFRGDSFTHTPPL | tEGFR |
| | DPQELDILKTVKEITGFLLIQAWPENRTDLHAFENLEIIRGRTKQHGQFSLAVV | artificial |
| | SLNITSLGLRSLKEISDGDVIISGNKNLCYANTINWKKLFGTSGQKTKIISNRG | |
| | ENSCKATGQVCHALCSPEGCWGPEPRDCVSCRNVSRGRECVDKCNLLEGEPREF | |
| | BENSECIQCHPECLPQAMNITCTGRGPDNCIQCAHYIDGPHCVKTCPAGVMGEN | |
| | NTLVWKYADAGHVCHLCHPNCTYGCTGPGLEGCPTNGPKIPSIATGMVGALLLL | |
| | LVVALGIGLFM | |
| 17 | EGRGSLLTCGDVEENPGP | T2A artificial |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | PGGG-(SGGGG)5-P- wherein P is proline, G is glycine and | linker |
| | S is serine | |
| 23 | GSADDAKKDAAKKDGKS | Linker |
| 24 | GSTSGSGKPGSGEGSTKG | Linker |
| 25 | gacatccagatgacccagaccacctccagcctgagcgccagcctgggcgaccgg | Sequence |
| | gtgaccatcagctgccgggccagccaggacatcagcaagtacctgaactggtat | encoding scFv |
| | cagcagaagcccgacggcaccgtcaagctgctgatctaccacaccagccggctg | |
| | cacagcggcgtgcccagccggtttagcggcagcggctccggcaccgactacagc | |
| | ctgaccatctccaacctggaacaggaagatatcgccacctacttttgccagcag | |
| | ggcaacacactgccctacacctttggcggcggaacaaagctggaaatcaccggc | |
| | agcacctccggcagcggcaagcctggcagcggcgagggcagcaccaagggcgag | |
| | gtgaagctgcaggaaagcggccctggcctggtggcccccagccagagcctgagc | |
| | gtgacctgcaccgtgagcggcgtgagcctgcccgactacggcgtgagctggatc | |
| | cggcagccccccaggaagggcctggaatggctgggcgtgatctggggcagcgag | |
| | accacctactacaacagcgccctgaagagccggctgaccatcatcaaggacaac | |
| | agcaagagccaggtgttcctgaagatgaacagcctgcagaccgacgacaccgcc | |
| | atctactactgcgccaagcactactactacggcggcagctacgccatggactac | |
| | tggggccagggcaccagcgtgaccgtgagcagc | |
| 26 | X₁PPX₂P | Hinge |
| | X₁ is glycine, cysteine or arginine | |
| | X₂ is cysteine or threonine | |
| 27 | Glu Pro Lys Ser Cys Asp Lys Thr His Thr Cys Pro Pro | Hinge |
| | Cys Pro | |
| 28 | Glu Arg Lys Cys Cys Val Glu Cys Pro Pro Cys Pro | Hinge |
| 29 | ELKTPLGDTHTCPRCPEPKSCDTPPPCPRCPEPKSCDTPPPCPRCPEPKSCDTP | Hinge |
| | PPCPRCP | |
| 30 | Glu Ser Lys Tyr Gly Pro Pro Cys Pro Ser Cys Pro | Hinge |
| 31 | Glu Ser Lys Tyr Gly Pro Pro Cys Pro Pro Cys Pro | Hinge |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro Cys | Hinge |
| | Pro | |
| 35 | RASQDISKYLN | FMC63 CDR L1 |
| 36 | SRLHSGV | FMC63 CDR L2 |
| 37 | GNTLPYTFG | FMC63 CDR L3 |
| 38 | DYGVS | FMC63 CDR H1 |
| 39 | VIWGSETTYYNSALKS | FMC63 CDR H2 |
| 40 | YAMDYWG | FMC63 CDR H3 |
| 41 | EVKLQESGPGLVAPSQSLSVTCTVSGVSLPDYGVSWIRQPPRKGLEWLGVIWGS | FMC63 VH |
| | ETTYYNSALKSRLTIIKDNSKSQVFLKMNSLQTDDTAIYYCAKHYYYGGSYAMD | |
| | YWGQGTSVTVSS | |
| 42 | DIQMTQTTSSLSASLGDRVTISCRASQDISKYLNWYQQKPDGTVKLLIYHTSRL | FMC63 VL |
| | HSGVPSRFSGSGSGTDYSLTISNLEQEDIATYFCQQGNTLPYTFGGGTKLEIT | |
| 43 | DIQMTQTTSSLSASLGDRVTISCRASQDISKYLNWYQQKPDGTVKLLIYHTSRL | FMC63 scFv |
| | HSGVPSRFSGSGSGTDYSLTISNLEQEDIATYFCQQGNTLPYTFGGGTKLEITG | |
| | STSGSGKPGSGEGSTKGEVKLQESGPGLVAPSQSLSVTCTVSGVSLPDYGVSWI | |
| | RQPPRKGLEWLGVIWGSETTYYNSALKSRLTIIKDNSKSQVFLKMNSLQTDDTA | |
| | IYYCAKHYYYGGSYAMDYWGQGTSVTVSS | |
| 44 | KASQNVGTNVA | SJ25C1 CDR L1 |
| 45 | SATYRNS | SJ25C1 CDR L2 |
| 46 | QQYNRYPYT | SJ25C1 CDR L3 |
| 47 | SYWMN | SJ25C1 CDR H1 |
| 48 | QIYPGDGDTNYNGKFKG | SJ25C1 CDR H2 |
| 49 | KTISSVVDFYFDY | SJ25C1 CDR H3 |
| 50 | EVKLQQSGAELVRPGSSVKISCKASGYAFSSYWMNWVKQRPGQGLEWIGQIYPG | SJ25 C1 VH |
| | DGDTNYNGKFKGQATLTADKSSSTAYMQLSGLTSEDSAVYFCARKTISSVVDFY | |
| | FDYWGQGTTVTVSS | |
| 51 | DIELTQSPKFMSTSVGDRVSVTCKASQNVGTNVAWYQQKPGQSPKPLIYSATYR | SJ25C1 VL |
| | NSGVPDRFTGSGSGTDFTLTITNVQSKDLADYFCQQYNRYPYTSGGGTKLEIKR | |
| 52 | GGGGSGGGGSGGGGS | Linker |
| 53 | EVKLQQSGAELVRPGSSVKISCKASGYAFSSYWMNWVKQRPGQGLEWIGQIYPG | SJ25C1 scFV |
| | DGDTNYNGKFKGQATLTADKSSSTAYMQLSGLTSEDSAVYFCARKTISSVVDFY | |
| | FDYWGQGTTVTVSSGGGGSGGGGSGGGGSDIELTQSPKFMSTSVGDRVSVTCKA | |
| | SQNVGTNVAWYQQKPGQSPKPLIYSATYRNSGVPDRFTGSGSGTDFTLTITNVQ | |
| | SKDLADYFCQQYNRYPYTSGGGTKLEIKR | |
| 54 | HYYYGGSYAMDY | FMC63 CDR H3 |
| 55 | HTSRLHS | FMC63 CDR L2 |
| 56 | QQGNTLPYT | FMC63 CDR L3 |

1. A method for expanding genetically engineered cells, comprising effecting a treatment, to a subject having a disease or condition, wherein the treatment comprises one or more of administration of an immunomodulatory agent, radiation, or a physical or mechanical manipulation of a lesion or portion thereof, said subject having previously received administration of the genetically engineered cells for treating a disease or condition, wherein the method results in expansion of the genetically engineered cells in the subject, in the lesion, and/or in a tissue or organ or fluid of the subject and/or in an increased number of the genetically engineered cells in the lesion, tissue or organ or fluid.

2. The method of claim 1, wherein the method does not comprise a subsequent administration of genetically engineered cells and/or the expansion is achieved without such a subsequent administration of the genetically engineered cells.

3. The method of claim 1 or claim 2, wherein, in the lesion or portion thereof, the engineered cells are present or likely to be present or were present or were likely to be present.

4. The method of any of claims 1-3, wherein the lesion is a tumor.

5. The method of claim 4, wherein the tumor is a primary or secondary tumor.

6. The method of claim any of claims 1-5, wherein the lesion is or comprises bone marrow tissue.

7. The method of any of claims 1, 2 and 4-6, wherein at or immediately prior to the time of the treatment, the subject has relapsed after response to the genetically engineered cells, optionally after remission, and/or did not respond to the administration of the genetically engineered cells.

8. The method of any one of claims 1-7, wherein the subject has relapsed after response to, and/or did not respond to, the previous administration of the genetically engineered cells.

9. The method of any one of claims 1-7, wherein the subject had responded to the genetically engineered cells and has subsequently ceased to respond and/or relapsed prior to the treatment.

10. The method of any one of claims 1-9, wherein the genetically engineered cells have previously expanded in the subject or been observed to have expanded prior to the treatment.

11. The method of any of claims 1-10, wherein at or immediately prior to the time of the treatment:
the subject is in remission;
the number of genetically engineered cells detectable in the blood is reduced or is not detectable;
the number of genetically engineered cells detectable in a fluid or tissue or sample, optionally the blood, from the subject is decreased compared to a preceding time point after administration of the genetically engineered cells; and/or
the number of cells of the genetically engineered cells detectable in a fluid or tissue or sample, optionally the blood, from the subject, is decreased by or more than 1.5-fold, 2.0-fold, 3.0-fold, 4.0-fold, 5.0-fold, 10-fold or more as compared to the peak or maximum number of the genetically engineered cells detectable or detected in the blood of the subject after initiation of administration of the genetically engineered cells and/or compared to the level at a time point within 1, 2, 3, 4, 5, 6, 7, 8, 9, 10, 11, 12, 13, or 14 or 28 days following the administration of the genetically engineered cells.

12. The method of any of claims 1-11, wherein the treatment is carried out at, at about, or greater than, or greater than about 2 weeks, 1 month, 2 months, 3 months, 4 months, 5 months, 6 months, 1 year or more after initiation of administration of the genetically engineered cells or after the last dose of the genetically engineered cells.

13. The method of any of claims 1-12, wherein the treatment directly or indirectly modulates an activity or function of the genetically engineered T cells in vivo in the subject.

14. The method of any of claims 1-13, wherein the treatment comprises administration of an immunomodulatory agent.

15. The method of claim 14, wherein the immunomodulatory agent is or comprises an immune-inhibitory molecule, is or comprises an immune checkpoint molecule or member of an immune checkpoint pathway and/or is or comprises a modulator of an immune checkpoint molecule or pathway.

16. The method of claim 15, wherein the immune checkpoint molecule or pathway is or comprises PD-1, PD-L1, PD-L2, CTLA-4, LAG-3, TIM3, VISTA, an adenosine receptor, CD73, CD39, adenosine 2A Receptor (A2AR), or adenosine or a pathway involving any of the foregoing.

17. The method of any of claims 1-16, wherein the immunomodulatory agent is BY55, MSB0010718C, ipilimumab, Daclizumab, Bevacizumab, Basiliximab, Ipilimumab, Nivolumab, pembrolizumab, MPDL3280A, Pidilizumab, MK-3475, BMS-936559, Atezolizumab, tremelimumab, IMP321, BMS-986016, LAG525, urelumab, PF-05082566, TRX518, MK-4166, dacetuzumab, lucatumumab, SEA-CD40, CP-870, CP-893, MEDI6469, MEDI6383, MOXR0916, AMP-224, Avelumab, MEDI4736, PDR001, rHIgM12B7, Ulocuplumab, BKT140, Varlilumab, ARGX-110, MGA271, lirilumab, IPH2201, ARGX-115, Emactuzumab, CC-90002 and MNRP1685A or an antibody-binding fragment thereof.

18. The method of any of claims 1-17, wherein the immunomodulatory agent is an anti-PD-L1 antibody.

19. The method of claim 1-18, wherein the anti-PD-L1 antibody is MEDI14736, MDPL3280A, BMS-936559, LY3300054, atezolizumab or avelumab or is an antigen-binding fragment thereof.

20. The method of claim 14, wherein the immunomodulatory agent is thalidomide or is a derivative or analogue of thalidomide.

21. The method of claim 14 or 20, wherein the immunomodulatory agent is lenalidomide or pomalidomide, avadomide, a stereoisomer of lenalidomide, pomalidomide, avadomide or a pharmaceutically acceptable salt, solvate, hydrate, co-crystal, clathrate, or polymorph thereof.

22. The method of any of claims 14, 20 and 21, wherein the immunomodulatory agent is lenalidomide, a stereoisomer of lenalidomide or a pharmaceutically acceptable salt, solvate, hydrate, co-crystal, clathrate, or polymorph thereof.

23. The method of any of claims 8-22, wherein after the relapse and prior to the treatment, the subject has not been administered an exogenous or recombinant agent for treating the disease or condition or for modulating the activity of the genetically engineered cells.

24. The method of any of claims 1-14 and 23, wherein the treatment comprises radiation.

25. The method of any of claims 1-14 and 23, wherein the treatment comprises a physical or mechanical manipulation of the lesion or a portion thereof, optionally wherein the physical or mechanical manipulation comprises penetrating an area of the lesion or portion thereof.

26. The method of claim 25, wherein the physical or mechanical manipulation comprises a biopsy.

27. The method of claim 26, wherein the biopsy is carried out by a needle or a trocar.

28. The method of claim 26 or claim 27, wherein the biopsy comprises an incisional biopsy.

29. The method of any of claims 1-28, wherein the method results in expansion of the genetically engineered cells or an increase in the number of the genetically engineered cells compared to at the time just prior to the treatment.

30. The method of any of claims 1-29, wherein expansion of the genetically engineered cells occurs within or within about 24 hours, 48 hours, 96 hours, 7 days, 14 days or 28 days after the treatment.

31. The method of any of claims 1-30, wherein:
the expansion results in greater than or greater than about 1.5-fold, 2.0-fold, 5.0-fold, 10-fold, 100-fold, 200-fold, or more genetically engineered cells detectable in the blood compared to just prior to the treatment; or
the expansion results in greater than or greater than about 1.5-fold, 2.0-fold, 5.0-fold, 10-fold, 100-fold, 200-fold, or more genetically engineered cells detectable in the blood compared to the prior peak levels of engineered cells in the blood prior to the treatment.

32. The method of any of claims 1-31, wherein the number of genetically engineered cells detectable in the blood at a time after the treatment is:
increased, optionallyincreased by 1.5-fold, 2-fold, 3-fold, 4-fold, 5-fold, 10-fold or more,compared to the number of the genetically engineered cells at a preceding time point before the treatment;
more than 1.5-fold 2-fold, 3-fold, 4-fold, 5-fold, 10-fold, 50-fold, 100-fold or more than the peak or maximum number of the genetically engineered cells detectable in the blood of the subject before the treatment;
more than or about more than 10%, 9%, 8%, 7%, 6%, 5%, 4%, 3%, 2%, 1%, 0.5%, 0.2% or 0.1% of the genetically engineered cells are detectable in the blood at a time after a peak of maximum level of such cells has been detected in the blood.

33. The method of any of claims 1-32, wherein the engineered cells express a recombinant receptor.

34. The method of claim 33, wherein the recombinant receptor specifically binds to an antigen associated with the disease or condition or expressed in cells of the lesion or a portion thereof.

35. The method of claim 34, wherein the antigen is selected from among 5T4, 8H9, avb6 integrin, B7-H6, B cell maturation antigen (BCMA), CA9, a cancer-testes antigen, carbonic anhydrase 9 (CAIX), CCL-1, CD19, CD20, CD22, CEA, hepatitis B surface antigen, CD23, CD24, CD30, CD33, CD38, CD44, CD44v6, CD44v7/8, CD123, CD138, CD171, carcinoembryonic antigen (CEA), CE7, a cyclin, cyclin A2, c-Met, dual antigen, EGFR, epithelial glycoprotein 2 (EPG-2), epithelial glycoprotein 40 (EPG-40), EPHa2, ephrinB2, erb-B2, erb-B3, erb-B4, erbB dimers, EGFR vIII, estrogen receptor, Fetal AchR, folate receptor alpha, folate binding protein (FBP), FCRL5, FCRH5, fetal acetylcholine receptor, G250/CAIX, GD2, GD3, gp100, Her2/neu (receptor tyrosine kinase erbB2), HMW-MAA, IL-22R-alpha, IL-13 receptor alpha 2 (IL-13Ra2), kinase insert domain receptor (kdr), kappa light chain, Lewis Y, L1-cell adhesion molecule (L1-CAM), Melanoma-associated antigen (MAGE)-A1, MAGE-A3, MAGE-A6, MART-1, mesothelin, murine CMV, mucin 1 (MUC1), MUC16, NCAM, NKG2D, NKG2D ligands, NY-ESO-1, O-acetylated GD2 (OGD2), oncofetal antigen, Preferentially expressed antigen of melanoma (PRAME), PSCA, progesterone receptor, survivin, ROR1, TAG72, tEGFR, VEGF receptors, VEGF-R2, Wilms Tumor 1 (WT-1), a pathogen-specific antigen.

36. The method of any of claims 1-35, wherein the disease or condition is a tumor or a cancer.

37. The method of any of claims 1-36, wherein the disease or condition is a leukemia or lymphoma.

38. The method of any of claims 1-37, wherein the disease or condition is a B cell malignancy.

39. The method of any of claims 1-38, wherein the disease or condition is lymphoblastic leukemia (ALL), chronic lymphoblastic leukemia (CLL), acute myelogenous leukemia (AML), chronic myelogenous leukemia (CML), non-Hodgkin lymphoma (NHL), or Diffuse Large B-Cell Lymphoma (DLBCL), or a subtype of any of the foregoing.

40. The method of any of claims 33-39, wherein the recombinant receptor is a T cell receptor or a functional non-T cell receptor.

41. The method of any of claims 34-40, wherein the recombinant receptor is a chimeric antigen receptor (CAR).

42. The method of claim 41, wherein the CAR comprises an extracellular antigen-recognition domain that specifically binds to the antigen and an intracellular signaling domain comprising an ITAM.

43. The method of any of claims 34-42, wherein the antigen is CD19.

44. The method of claim 42 or claim 43, wherein the intracellular signaling domain comprises an intracellular domain of a CD3-zeta (CD3) chain.

45. The method of any of claims 41-44, wherein the CAR further comprises a costimulatory signaling region.

46. The method of claim 45, wherein the costimulatory signaling domain comprises a signaling domain of CD28 or 4-1BB.

47. The method of any of claims 1-46, wherein the genetically engineered cells comprise T cells or NK cells.

48. The method of any of claims 1-46, wherein the genetically engineered cells are T cells and the T cells are CD4+ or CD8+ T cells.

49. The method of any of claims 1-48, wherein the therapy cells of the genetically engineered T cells comprises primary cells derived from a subject.

50. The method of any of any of claims 1-49, wherein the cells of the genetically engineered cells are autologous to the subject.

51. The method of any of claims 1-49, wherein the cells of the genetically engineered cells are allogeneic to the subject.

52. The method of any of claims 1-51, wherein the subject is a human.

53. The method of any of claims 1-52, wherein the dose of genetically engineered cells previously administered comprises a dose from or from about 1×10⁵ to 5×10⁸ total recombinant receptor-expressing cells, total T cells, or total peripheral blood mononuclear cells (PBMCs), from or from about 1×10⁵ to 1×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive.

54. The method of any of claims 1-53, wherein the dose of genetically engineered cells previously administered is no more than 5×10⁸ total recombinant receptor-expressing cells, total T cells, or total peripheral blood mononuclear cells (PBMCs), no more than 1×10⁸ total recombinant receptor-expressing cells, total T cells, or total peripheral blood mononuclear cells (PBMCs), no more than 1×10⁷ total recombinant receptor-expressing cells, total T cells, or total peripheral blood mononuclear cells (PBMCs), no more than 0.5×10⁷ total recombinant receptor-expressing cells, total T cells, or total peripheral blood mononuclear cells (PBMCs), no more than 1×10⁶ total recombinant receptor-expressing cells, total T cells, or total peripheral blood mononuclear cells (PBMCs), no more than 0.5×10⁶ total recombinant receptor-expressing cells, total T cells, or total peripheral blood mononuclear cells (PBMCs).

55. The method of any of claims 1-54, wherein the dose of genetically engineered cells previously administered comprises a dose between about 0.25×10⁶ cells/kg body weight of the subject and 5×10⁶ cells/kg, 0.5×10⁶ cells/kg body weight of the subject and 3×10⁶ cells/kg, between about 0.75×10⁶ cells/kg and 2.5×10⁶ cells/kg or between about 1×10⁶ cells/kg and 2×10⁶ cells/kg, each inclusive.

56. The method of any of claims 1-55, wherein the dose of genetically engineered cells are administered in a single pharmaceutical composition comprising the cells or as a plurality of compositions together comprising the cells.

57. The method of any of claims 1-56, wherein the genetically engineered cells administered is a split dose, wherein the cells of the dose are administered in a plurality of compositions, collectively comprising the cells of the dose, over a period of no more than three days.

58. The method of any of claims 1-57, wherein the method comprises effecting a subsequent treatment, wherein the subsequent treatment comprises one or more of administration of an immunomodulatory agent, radiation, or a physical or mechanical manipulation of a lesion or portion thereof, optionally wherein the subsequent treatment is effected after the subject has relapsed following response after the preceding treatment and/or has not achieved a complete response after the preceding treatment.

59. The method of any claim 58, wherein the subject had responded to the genetically engineered cells after the preceding treatment and has subsequently ceased to respond and/or relapsed prior to the subsequent treatment.

60. The method of claim 58 or claim 59, wherein the genetically engineered cells have expanded in the subject or been observed to have expanded after the preceding treatment and prior to the subsequent treatment.

61. The method of any of claims 58-60, wherein at or immediately prior to the time of the subsequent treatment:
the subject is in remission;
the number of genetically engineered cells detectable in the blood is reduced or is not detectable;
the number of genetically engineered cells detectable in a fluid or tissue or sample, optionally the blood, from the subject is decreased compared to a preceding time point after initiation of the preceding treatment; and/or
the number of cells of the genetically engineered cells detectable in a fluid or tissue or sample, optionally the blood, from the subject, is decreased by or more than 1.5-fold, 2.0-fold, 3.0-fold, 4.0-fold, 5.0-fold, 10-fold or more as compared to the peak or maximum number of the genetically engineered cells detectable or detected in the blood of the subject after initiation of the preceding treatment and/or compared to the level at a time point within 1, 2, 3, 4, 5, 6, 7, 8, 9, 10, 11, 12, 13, or 14 or 28 days following initiation of the preceding treatment.

62. The method of any of claims 1-61, wherein the genetically engineered cells exhibit increased or prolonged expansion and/or persistence in the subject as compared to a method in which the genetically engineered cells are administered to the subject in the absence of the treatment.

63. The method of any of claims 1-62, wherein the method reduces tumor burden to a greater degree and/or for a greater period of time as compared to the reduction that would be observed with a comparable method in which the genetically engineered cells are administered to the subject in the absence of the treatment and/or in which the treatment is effected in the absence of the genetically engineered cells, optionally at the same dose or dosing schedule.
